(19) United States
(12) Patent Application Publication
(10) Pub. No.: US 2023/0393136 A1
(43) Pub. Date: Dec. 7, 2023

(54) URINARY GLYCOPROTEINS FOR THE EARLY DETECTION AND TREATMENT OF AGGRESSIVE PROSTATE CANCER

(71) Applicant: THE JOHNS HOPKINS UNIVERSITY, Baltimore, MD (US)

(72) Inventors: Hui Zhang, Ellicott City, MD (US); Mingming Dong, Baltimore, MD (US); Mamie Lih, Baltimore, MD (US)

(21) Appl. No.: 18/043,495

(22) PCT Filed: Aug. 30, 2021
(86) PCT No.: PCT/US2021/048234

§ 371 Date: Feb. 28, 2023

Related U.S. Application Data
(60) Provisional application No. 63/071,767, filed on Aug. 28, 2020.

(51) Int. Cl. G01N 33/574

(52) U.S. Cl. CPC G01N 33/57434; G01N 2333/4728; G01N 2333/70596; G01N 2333/916; G01N 2333/96455

(57) ABSTRACT

The present invention relates to the field of cancer. Specifically, the present invention provides compositions and methods useful for detecting and treating aggressive prostate cancer. In another embodiment, a method for identifying a subject as having aggressive prostate cancer comprises the step of measuring one or more of ACPP, CD63, KLK 11, ATRN, GP2, PTPRN2, NPTN, RNASE2, CPE, SERPINA1, DSC2, PTGDS, GRN, LRG1, UMOD, CLU, LOX, ORM1, CD97, PSA and AFM in a urine sample obtained from the subject, wherein a decreased level of one or more of ACPP, CD63, KLK 11, ATRN, GP2, PTPRN2, NPTN, RNASE2, PSA, CPE and/or an increased level of one or more of SERPINA1, DSC2, PTGDS, GRN, LRG1, UMOD, CLU, LOX, ORM1, CD97, and AFM relative to a control identifies the subject as having aggressive prostate cancer. In another embodiment, the method further comprises the step of treating the subject with a prostate cancer therapy.

### CROSS-REFERENCE TO RELATED APPLICATIONS

This application claims the benefit of U.S. Provisional Application No. 63/071,767, filed Aug. 28, 2020, which is incorporated herein by reference in its entirety.

### GOVERNMENT SUPPORT CLAUSE

This invention was made with government support under grant no. CA152813, awarded by the National Institutes of Health. The government has certain rights in the invention.

### FIELD OF THE INVENTION

The present invention relates to the field of cancer. More specifically, the present invention provides compositions and methods useful for detecting and treating aggressive prostate cancer.

### INCORPORATION-BY-REFERENCE OF MATERIAL SUBMITTED ELECTRONICALLY

This application contains a sequence listing. It has been submitted electronically via EFS-Web as an ASCII text file entitled “P15992-02_ST25.txt.” The sequence listing is 104,945 bytes in size, and was created on Aug. 30, 2021. It is hereby incorporated by reference in its entirety.

### BACKGROUND OF THE INVENTION

Prostate cancer (PCa) is the most frequently diagnosed cancer and the second leading cause of cancer-related death for men [1]. However, most patients presenting with PCa actually have a low-risk form (Gleason score=6) that does not require interventions including unnecessary biopsies and treatments [2]. Currently, there are no Food and Drug Administration (FDA)-approved noninvasive biomarkers that can be used to differentiate aggressive (AG) from non-aggressive (NAG) PCa. Therefore, discovering noninvasive biomarkers for AG PCa is crucial.

Urine is a promising specimen for the discovery of noninvasive biomarkers associated with PCa. Urine-derived genetic biomarkers from RNA and DNA or metabolites have been investigated for their diagnostic and prognostic value for PCa [3-13]. Urine-derived long non-coding RNA prostate cancer antigen-3 (PCA3) is the first U.S. FDA approved urinary biomarker for PCa to aid decision making for repeated biopsies [14], while its ability in detecting the

aggressiveness of PCa is limited [15]. A recent multi-center study assessed the diagnostic and prognostic values of PCA3 and TMPRSS2:ERG gene fusions for PCa, in which TMPRSS2:ERG gene fusions, other than PCA3, has demonstrated prognostic value for PCa [7].

Urinary miRNAs have been proposed as potential prognostic biomarkers for PCa and different panels of urinary miRNAs have been used to predict the aggressiveness of the cancer

with comparable performance to most tissue-based prognostic assays (AUC around 0.7) [11]. Besides miRNAs, urine proteins have also been investigated, for which moderate performance is observed in distinguishing the extracapsular stage pT3 PCa from the organ-confined stage pT2 PCa (AUC=0.74) [9]. Nonetheless, due to the heterogeneous nature of PCa [6], there is still no ideal marker for the detection of aggressive PCa. Thus, it is essential to discover novel biomarkers that can work independently or in combination with currently available biomarkers to improve the discrimination power towards aggressive PCa.

### SUMMARY OF THE INVENTION

Urine is a rich source for glycoproteins derived from the urogenital system, and the majority of aggressive prostate cancer-associated glycoproteins from prostate cancer tissues are more readily detected in the patient's urine than serum samples. The present inventors applied the glycoproteomic workflow to the analysis of urine specimens from 75 aggressive and 70 nonaggressive prostate cancer patients using mass spectrometry. The present inventors discovered several glycoproteins associated with aggressive prostate cancer and evaluated the candidate glycoproteins using receiver operating characteristic analysis and multivariate logistic regression models. In certain embodiments, glycopeptides from ACPP, CD63, KLK 11, ATRN, GP2, PTPRN2, NPTN, RNASE2, and CPE were found significantly decreased in urine specimens of aggressive prostate cancer. Furthermore, glycopeptides from SERPINAL DSC2, PTGDS, GRN, LRG1, UMOD, CLU, LOX, ORM1, CD97, and AFM were found significantly elevated in aggressive prostate cancer urine specimens. The urine glycoprotein tests showed improved performance for aggressive prostate cancer diagnosis using noninvasive urinary test for selection patients with aggressive prostate cancer for treatment by avoiding unnecessary biopsy.

Thus, in one aspect, the present invention provides assays useful for identifying patients as having or likely to have aggressive prostate cancer. The assays can be singleplex or multiplex assays. In certain embodiments, the assay utilizes antibodies to capture biomarker glycoproteins of interest. The assays can further use antibodies to detect and quantify biomarker glycoproteins of interest. The assay can also use lectins to enrich, isolate or otherwise select for the biomarker glycoproteins of interest followed by subsequent analysis using antibodies. In certain embodiments, the assay utilizes both antibodies and lectins to detect and measure biomarker glycoproteins of interest. In certain embodiments, the assay utilizes lectins or antibodies to enrich target glycoproteins and followed by mass spectrometric analysis. In certain embodiments, the assay utilizes mass spectrometry to quantify the glycoproteins.

In one embodiment, a method comprises the step of measuring one or more of prostatic acid phosphatase (ACPP), CD63 antigen (CD63), kallikrein-11 (KLK11), attractin (ATRN), pancreatic secretory granule membrane major glycoprotein GP2 (GP2), receptor-type tyrosine-protein phosphatase N2 (PTPRN2), neuroplastin (NPTN), non-secretory ribonuclease (RNASE2), prostate-specific antigen (PSA) (also known as kallikrein-3 (KLK3), carboxypeptidase E (CPE), alpha-1-antitrypsin (SERPINA1), desmocollin-2 (DSC2), prostaglandin-H2 D9 isomerase (PTGDS), progranulin (GRN), leucine-rich alpha-2-glycprotein (LRG1), uromodulin (UMOD), clusterin (CLU), protein-lysine 6-oxidase (LOX), alpha-1-acid glycoprotein 1 (ORM1), CD97 antigen (CD97), and afamin (AFM) in a urine sample obtained from a subject having or suspected of having prostate cancer. In another embodiment, the method further comprises the step of measuring prostate-specific antigen (PSA) in a serum sample obtained from the subject.

In particular embodiments, the measuring step comprises an immunoassay. In a specific embodiment, the immunoassay comprises enzyme linked immunosorbent assay (ELISA). In certain embodiments, the measuring step comprises mass spectrometry.

In another embodiment, a method for identifying a subject as having aggressive prostate cancer comprises the step of measuring one or more of ACPP, CD63, KLK 11, ATRN, GP2, PTPRN2, NPTN, RNASE2, PSA, CPE, SERPINA1, DSC2, PTGDS, GRN, LRG1, UMOD, CLU, LOX, ORM1, CD97, and AFM in a urine sample obtained from the subject, wherein a decreased level of one or more of ACPP, CD63, KLK 11, ATRN, GP2, PTPRN2, NPTN, RNASE2, PSA, and CPE, and/or an increased level of one or more of SERPINA1, DSC2, PTGDS, GRN, LRG1,UMOD, CLU, LOX, ORM1, CD97, and AFM relative to a control identifies the subject as having aggressive prostate cancer. In yet another embodiment, the method further comprises the step of treating the subject with a prostate cancer therapy.

In a specific embodiment, a method comprise the steps of (a) detecting an decreased level of one or more of ACPP, CD63, KLK 11, ATRN, GP2, PTPRN2, NPTN, RNASE2, PSA and CPE and/or an increased level of one or more of SERPINA1, DSC2, PTGDS, GRN, LRG1, UMOD, CLU, LOX, ORM1, CD97, and AFM, relative to a control in a urine sample obtained from a subject having or suspected of having prostate cancer; and (b) treating the subject with a prostate cancer therapy.

In another specific embodiment, a method for identifying a subject as having aggressive prostate cancer comprises the steps of (a) measuring ACPP and one or more of clusterin (CLU), protein-lysine 6-oxidase (LOX), alpha-1-antitrypsin (SERPINA1), and alpha-1-acid glycoprotein 1 (ORM1), in a urine sample obtained from the subject; and (b) correlating the levels measured in step (a) with serum PSA to identify the subject as having aggressive prostate cancer. In a more specific embodiment, the method further comprises the step of treating the subject with a prostate cancer therapy.

The present invention also provides a method comprising the step of measuring (a) urine ACPP and serum PSA; (b) urine ACPP and urine CLU; (c) urine ACPP and urine LOX; (d) urine ACPP and urine SERPINA1; (e) urine ACPP and urine ORM1; (f) ACPP, urine CLU and serum PSA; (g) urine ACPP, urine LOX and serum PSA; (h) urine ACPP, urine SERPINA1 and serum PSA; or (i) urine ACPP, urine ORM1 and serum PSA, in samples obtained from a subject having or suspected of having prostate cancer. In a specific embodiment, the subject is identified as having aggressive prostate cancer or non-aggressive prostate cancer based on the measured levels. In a more specific embodiment, the subject having aggressive prostate cancer is treated with a prostate cancer therapy.

In an alternative embodiment, a method comprises the step of measuring (a) ACPP; (b) ACPP and CLU; (c) ACPP and LOX; (d) ACPP and SERPINA1; (e) ACPP and ORM1; (f) ACPP and CLU; (g) ACPP and LOX; (h) ACPP and SERPINA1; or (i) ACPP and ORM1, in a urine sample obtained from a subject having or suspected of having prostate cancer. In a specific embodiment, the measured proteins are used with serum PSA to diagnose the subject as having aggressive prostate cancer or non-aggressive prostate cancer. In a more specific embodiment, the subject having aggressive prostate cancer is treated with a prostate cancer therapy.

In a further embodiment, a method comprises the step of measuring ACPP, CLU, ORM1, CD97, and/or PSA in a urine sample obtained from a subject having or suspected of having prostate cancer. In a more specific embodiment, the measured proteins are used with combination of any two, three, four, or five of them to diagnose the subject as having aggressive prostate cancer or non-aggressive prostate cancer. In an even more specific embodiment, the subject having aggressive prostate cancer is treated with a prostate cancer therapy.

In particular embodiments, the prostate cancer therapy comprises prostatectomy, radiation therapy, cryotherapy, hormone therapy, chemotherapy, immunotherapy and combinations thereof.

In particular embodiments, the measuring step comprises an immunoassay. In a specific embodiment, the immunoassay comprises enzyme linked immunosorbent assay (ELISA). In certain embodiments, the measuring step comprises mass spectrometry.

The methods described herein can be used to measure the biomarker proteins and/or modified forms thereof including glycosylated forms. In some embodiments, the assays herein can be used or adjusted to measure total levels of a given protein and/or levels of a modified version of the protein. Total glycoprotein levels, specific levels of glycoproteins and/or the like can be measured.

In one embodiment, a method comprises (a) isolating glycoproteins from a urine sample obtained from the patient using a lectin affinity capture assay; and (b) quantitating the amount of a panel of glycoproteins from the isolated glycoproteins of step (a) using an immunoassay. In further embodiments, the method can further comprise the step of identifying the patient as having aggressive prostate cancer. In certain embodiments, the method further comprises the step of recommending, prescribing or treating the patient with, an appropriate therapeutic regimen for aggressive prostate cancer. It is also contemplated that the methods comprise a recommendation, prescription, treatment or administration of a cancer therapy to a patient identified or diagnosed as having aggressive prostate cancer or non-aggressive prostate cancer using a method described herein.

In one aspect, the present invention provides multiplex assays for distinguishing aggressive from non-aggressive prostate cancer in a patient. In particular embodiments, the method comprises the steps of (a) incubating a sample comprising biomarker glycoproteins of interest obtained from a patient with a plurality of lectins that specifically bind glycoproteins; (b) adding a plurality of monoclonal antibodies that specifically bind the biomarker glycoproteins of interest; (c) detecting the lectin-bound biomarker glycoproteins using a labeled detection antibody that binds the lectin-bound biomarker glycoproteins; and (d) identifying the patient as having aggressive prostate cancer if the detected biomarker glycoproteins are statistically significantly changed relative to reference levels that correlate to non-aggressive prostate cancer. In a specific embodiment, the biomarker glycoproteins of interest comprise one or more of ACPP, CD63, KLK 11, ATRN, GP2, PTPRN2, NPTN, RNASE2, PSA, CPE, SERPINA1, DSC2, PTGDS, GRN, LRG1, UMOD, CLU, LOX, ORM1, CD97, and AFM.

In a specific embodiment, a multiplex assay for distinguishing aggressive from non-aggressive prostate cancer in a patient comprises the steps of (a) incubating a urine sample comprising biomarker glycoproteins of interest obtained from a patient with a plurality of lectins that specifically bind glycoproteins, wherein the biomarker glycoproteins of interest comprise one or more of ACPP, CD63, KLK 11, ATRN, GP2, PTPRN2, NPTN, RNASE2, PSA, CPE, SERPINA1, DSC2, PTGDS, GRN, LRG1, UMOD, CLU, LOX, ORM1, CD97, and AFM; (b) adding a plurality of monoclonal antibodies that specifically bind the biomarker glycoproteins of interest; (c) detecting the lectin-bound biomarker glycoproteins using a labeled detection antibody that binds the lectin-bound biomarker glycoproteins; and (d) identifying the patient as having aggressive prostate cancer if the detected biomarker glycoproteins are statistically significantly changed relative to reference levels that correlate to non-aggressive prostate cancer or no cancer.

In other embodiments, a multiplex assay for distinguishing aggressive from non-aggressive prostate cancer in a patient comprises the steps of (a) incubating a sample comprising biomarker glycoproteins of interest obtained from a patient with a plurality of binding agents that specifically bind the biomarker glycoproteins of interest; (b) detecting the biomarker proteins using an immunoassay or mass spectrometry; and (d) identifying the patient as having aggressive prostate cancer if there is a statistically significant difference in the levels of the detected biomarker glycoproteins as compared to corresponding levels in a control sample that correlates to non-aggressive prostate cancer. In a specific embodiment, the biomarker glycoproteins of interest comprise one or more of ACPP, CD63, KLK 11, ATRN, GP2, PTPRN2, NPTN, RNASE2, PSA, CPE, SERPINA1, DSC2, PTGDS, GRN, LRG1, UMOD, CLU, LOX, ORM1, CD97, and AFM.

In another aspect, the present invention provides methods for treating prostate cancer in a patient. In a specific embodiment, the method comprises the steps of (a) collecting a urine sample from the patient; (b) detecting the levels of a panel of biomarkers in the sample collected from the patient, wherein the panel of biomarkers comprises one or more of ACPP, CD63, KLK 11, ATRN, GP2, PTPRN2, NPTN, RNASE2, PSA, CPE, SERPINA1, DSC2, PTGDS, GRN, LRG1, UMOD, CLU, LOX, ORM1, CD97, and AFM; (c) comparing the levels of the panel of biomarkers with predefined levels of the same panel of biomarkers that correlate to a patient having aggressive prostate cancer and predefined levels of the same panel of biomarkers that correlate to a patient not having aggressive prostate cancer, wherein a correlation to one of the predefined levels provides the diagnosis; and (d) treating the patient with an appropriate therapeutic regimen for aggressive prostate cancer if the diagnosis of the patient correlates to aggressive prostate cancer or treating the patient with an appropriate therapeutic regimen for non-aggressive prostate cancer if the diagnosis of the patient correlates to non-aggressive prostate cancer. The appropriate therapeutic regimen (for aggressive prostate cancer or for non-aggressive prostate cancer) can be determined by one of ordinary skill in the art using the methods described herein and other patient and diagnostic information.

In any of the embodiments recited herein, the biomarker glycoproteins of interest comprise ACPP and one or more of CD63, KLK 11, ATRN, GP2, PTPRN2, NPTN, RNASE2, PSA, CPE, SERPINA1, DSC2, PTGDS, GRN, LRG1, UMOD, CLU, LOX, ORM1, CD97, and AFM. In yet another embodiment, the biomarker glycoproteins of interest comprise ACPP and one or more of DSC2, PTGDS, GRN, AFM, CD97, LRG1, and UMOD. In a further embodiment, the biomarker glycoproteins of interest comprise ACPP and one or more of SERPINA1, CLU, LOX, and ORM1. In certain embodiments, the biomarker glycoproteins of interest comprise one or more of CD63, KLK 11, ATRN, GP2, PTPRN2, NPTN, RNASE2, PSA, CPE and one or more of SERPINA1, DSC2, PTGDS, GRN, LRG1, UMOD, CLU, LOX, ORM1, CD97, and AFM.

In one embodiment, a panel of biomarkers comprises urine ACPP and serum PSA. In another embodiment, a panel of biomarkers comprises urine ACPP and urine CLU. In yet another embodiment, a panel of biomarkers comprises urine ACPP and urine LOX. In a specific embodiment, a panel of biomarkers comprises urine ACPP and urine SERPINA1. In another specific embodiment, a panel of biomarkers comprises urine ACPP and urine ORM1.

In one embodiment, a panel of biomarkers comprises urine ACPP, urine CLU and serum PSA. In another embodiment, a panel of biomarkers comprises ACPP, urine LOX and serum PSA. In a specific embodiment, a panel of biomarkers comprises urine ACPP, urine SERPINA1 and serum PSA. In another specific embodiment, a panel of biomarkers comprises urine ACPP, urine ORM1 and serum PSA. It is further contemplated that panels of biomarkers can comprise the markers described in the figures and/or tables described herein.

In certain embodiments, the present invention comprises detection of the glycoproteins described herein, and more specifically, the glycoproteins comprising the following glycopeptides (Protein (glycopeptide sequence (*denotes glycosite)) (Uniprot No.): ACPP (FLN*ESYK) (SEQ ID NO:1) (P15309); LOX (AEN*QTAPGEVPALSNLRPPSR) (SEQ ID NO:2) (P28300); CLU (EDALN*ETR) (SEQ ID NO:3) (P10909); SERPINA1 (YLGN*ATAIFFLPDEGK) (SEQ ID NO:4) (P01009); ORM1 (QDQCIYN*TTYLNVQR) (SEQ ID NO:5) (P02763); CD63 (CCGAAN*YTDWEK) (SEQ ID NO:6) (P08962); ATRN (ISN*SSDTVECECSENWK) (SEQ ID NO:7) (075882); GP2 (QDLN*SSDVHSLQPQLDCGPR) (SEQ ID NO:8) (P55259); KLK11 (TATESFPHPGFN*NSLPNK) (SEQ ID NO:9) (Q9UBX7); PTPRN2 (VSANVQN*VTTEDVEK) (SEQ ID NO:10) (Q92932); NPTN (AN*ATIEVK) (SEQ ID NO:11) (Q9Y639); CPE (DLQGNPIAN*ATISVEGIDHDVTSAK) (SEQ ID NO:12) (P16870); RNASE2 (NQNTFLLTTFANVVNVCGNPN*MTCPSN*K) (SEQ ID NO:13) (P10153); DSC2 (NGIYN*ITVLASDQGGR) (SEQ ID NO:14) (Q02487); LRG1 (LPPGLLAN*FTLLR) (SEQ ID NO:15) (P02750); GRN (DVECGEGHFCHDN*QTCCR) (SEQ ID NO:16) (P28799); PTGDS (SVVAPATDGGLN*LTSTFLR) (SEQ ID NO:17) (P41222); UMOD (QDFN*ITDISLLEHR) (SEQ ID NO:18) (P07911); AFM (DIENFN*STQK) (SEQ ID NO:19) (P43652); CD97 (WCPQNSSCVN*ATACR) (SEQ ID NO:20) (P48960); ORM1 (QDQCIYN*TTYLNVQR) (SEQ ID NO:21) (P02763); and TMPRSS2 (LN*TSAGNVDIYK) (SEQ ID NO:21) (O15393).

Embodiments in which the specific biomarkers proteins are recited (e.g., ACPP, LOX and the like) also refers specifically to the peptides recited above (e.g., ACPP (FLN*ESYK) (SEQ ID NO:1), LOX (AEN*QTAPGEVPALSNLRPPSR) (SEQ ID NO:2), and the like). Capture/detection agents (e.g., antibodies, lectins, etc.) specific for these peptides are contemplated within relevant embodiments.

### BRIEF DESCRIPTION OF THE FIGURES

FIG. 1A-1C. FIG. 1A: Experimental workflow for the quantitative analysis of urine glycoproteomic to discover candidate biomarkers associated with aggressive prostate cancer. Reproducibility of DIA MS analysis was shown. FIG. 1B: The relative standard deviation (RSD) of the identification number of peptide precursors, peptides and proteins over three replicate DIA runs of glycopeptides are less than 3%. FIG. 1C: The correlation coefficients between any two replicates was at least 0.944.

FIG. 2. Identifications of 79 glycopeptides with significant fold change between AG and NAG samples (p<0.05). Glycopeptides with elevated levels in AG samples and NAG samples are in red and blue, respectively. The right panel shows the fold change of the glycopeptides between AG and NAG samples.

FIG. 3A-3F. Two down-regulated glycopeptides in AG PCa. FIG. 3A: Expression profiles of urinary ACPP (FLN*ESYK) (SEQ ID NO:1) in AG PCa and NAG PCa samples. FIG. 3B: ROC analysis results of urinary ACPP and serum PSA. FIG. 3C: A panel comprising urinary glycopeptide from ACPP and serum PSA was evaluated by label permutation for 1000 times. The AUC distribution of the 1000 random models (median AUC=0.55, red dotted line) was compared to the real model (AUC=0.82, black dotted line). FIG. 3D: Effect of serum PSA concentrations on the performance of urinary ACPP for detecting AG PCa. The AUC of urinary ACPP and serum PSA for detecting AG PCa was calculated and compared at different serum PSA cutoffs. FIG. 3E: Expression profiles of CD63 (CCGAAN*YTDWEK) (SEQ ID NO:6) in AG PCa and NAG PCa urine samples. FIG. 3F: ROC analysis results of CD63 (CCGAAN*YTDWEK) (SEQ ID NO:6) and serum PSA. The boxplots display a summary of minimum, first quartile, median, third quartile, and maximum of the expression profiles for AG and NAG PCa samples. AUC and 95% confidence interval are depicted for each candidate marker.

FIG. 4A-4F. Three up-regulated glycopeptides in AG PCa. FIG. 4A: Expression profiles of DSC2 (NGIYN*ITVLASDQGGR) (SEQ ID NO:14) in AG and NAG PCa urine samples. FIG. 4B: ROC analysis results of DSC2 (NGIYN*ITVLASDQGGR) (SEQ ID NO:14) and serum PSA. FIG. 4C: Expression profiles of LOX (AEN*QTAPGEVPALSNLRPPSR) in AG PCa and NAG PCa urine samples. FIG. 4D: ROC analysis results of LOX (AEN*QTAPGEVPALSNLRPPSR) (SEQ ID NO:2) and serum PSA. FIG. 4E: Expression profiles of LRG1 (LPPGLLAN*FTLLR) (SEQ ID NO:15) in AG PCa and NAG PCa urine samples. FIG. 4F: ROC analysis results of LRG1 (LPPGLLAN*FTLLR) (SEQ ID NO:15) and serum PSA. The boxplots display a summary of minimum, first quartile, median, third quartile, and maximum of the expression profiles for AG and NAG PCa samples. AUC and 95% confidence interval are depicted for each candidate marker.

FIG. 5A-5D. ROC analysis of combined panels including urinary ACPP (FLN*ESYK) (SEQ ID NO:1), one up-regulated glycopeptide, and serum PSA. FIG. 5A: The combinatory performance of ACPP (FLN*ESYK) (SEQ ID NO:1), CLU (EDALN*ETR) (SEQ ID NO:3) and serum PSA. FIG. 5B: The combinatory performance of ACPP (FLN*ESYK) (SEQ ID NO:1), LOX (AEN*QTAPGEVPALSNLRPPSR) (SEQ ID NO:2) and serum PSA. FIG. 5C: The combinatory performance of ACPP (FLN*ESYK) (SEQ ID NO:1), SERPINA1 (YLGN*ATAIFFLPDEGK) (SEQ ID NO:4) and serum PSA. FIG. 5D: The combinatory performance of ACPP (FLN*ESYK) (SEQ ID NO:1), ORM1 (QDQCIYN*TTYLNVQR) (SEQ ID NO:5) and serum PSA.

FIG. 6. Schematic overview of candidate glycopeptide discovery and validation.

FIG. 7. Schematic overview of the experimental workflow. Urinary glycopeptides were first isolated from the clinical samples using an automated high-throughput method. The PRM assays were developed using heavy isotope-labeled peptides and the analytical performances were determined. The isolated glycopeptides along with the spike-in heavy isotope-labeled peptides were quantified using the newly established targeted PRM assays. The quantification results were statistically analyzed and the clinical performance of the candidate peptides in detecting aggressive PCa were investigated and further evaluated using a second cohort.

FIG. 8A-8F. The analytical performance of the established PRM assays for glycopeptides from urinary CLU and ACPP. FIG. 8A: Reversed calibration curves for FLN*ESYK from ACPP. FIG. 8B: The extracted ion chromatography for the PRM transitions of heavy isotope-labeled peptide FLN*ESYK[+8] (SEQ ID NO:1) from ACPP. FIG. 8C: The extracted ion chromatography of PRM transitions of endogenous peptide FLN*ESYK (SEQ ID NO:1) from ACPP. FIG. 8D: Reversed calibration curves for glycopeptide EDALN*ETR (SEQ ID NO:3) from CLU. FIG. 8E: The extracted ion chromatography of the PRM transitions of heavy-isotope labeled peptide EDALN*ETR[±10] (SEQ ID NO:3) from CLU. FIG. 8F: The extracted ion chromatography of the PRM transitions of endogenous peptide EDALN*ETR (SEQ ID NO:3) from CLU. # Calibration curves of the triplicate along with their average are plotted. The linear regression model and R² values were calculated based on the average calibration curve.

FIG. 9A-9C. Reproducibility of the established PRM assays. FIG. 9A: PRM analysis of the targeted glycopeptides from ACPP was performed in four replicates. The intensities (peak area) of the top four PRM transitions for heavy isotope-labeled peptide FLN*ESYK[+8] (SEQ ID NO:1) and endogenous peptide FLN*ESYK (SEQ ID NO:1) from ACPP across the four replicates are displayed in (i) and (ii), respectively. FIG. 9B: The intensities of the top four PRM transitions for heavy-isotope labeled peptide EDALN*ETR[±10] (SEQ ID NO:3) and endogenous peptide EDALN*ETR (SEQ ID NO:3) from CLU across the four replicates are displayed in (i) and (ii), respectively. FIG. 9C: The data points from the calibration curves of the triplicate (data was collected across 5 days) were used to assess the repeatability of the PRM assays. The CVs of the measured heavy and light ratios at different heavy isotope-labeled peptide spike in levels are plotted. #H/L ratio is the ratio between heavy isotope-labeled peptide and endogenous peptide.

FIG. 10A-10E. Expression of ACPP and CLU based on the ratio of endogenous glycopeptides to heave-isotope labeled peptides and their predictive power towards aggressive PCa via the established PRM assays along with serum PSA. FIG. 10A: Expression profiles of the glycopeptide from ACPP between aggressive (Gleason 8, n=73) and non-aggressive (Gleason 6, n=69) PCa groups. FIG. 10B: Expression profiles of glycopeptide from CLU between aggressive (Gleason 8, n=73) and non-aggressive (Gleason 6, n=69) PCa groups. FIG. 10C: Expression profiles of serum PSA between aggressive (Gleason 8, n=73) and non-aggressive (Gleason 6, n=69) PCa groups. FIG. 10D: The performance of urinary ACPP, urinary CLU and serum PSA in detecting aggressive PCa individually. FIG. 10E: The performance of urinary ACPP, urinary CLU, or serum PSA test combined into as two- or three-signature panels.

FIG. 11. Performance of urinary ACPP, urinary CLU, and urine PSA in detecting aggressive PCa individually and in combination.

FIG. 12A-12B. Evaluation of the predictive models in comparison to random models. The panels were evaluated by label permutation for 500 times. The AUC distribution of the 500 random models was compared to the real models. FIG. 12A: panel comprising urinary glycopeptides from ACPP and CLU. FIG. 12B: panel composed of urinary glycopeptides from ACPP and CLU along with urine PSA.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

There is an urgent need for the detection of aggressive prostate cancer. Glycoproteins play essential roles in cancer development, while urine is a noninvasive and easily obtainable biological fluid that contains secretory glycoproteins from the urogenital system. Therefore, the present inventors aimed to identify urinary glycoproteins that are capable of differentiating aggressive from non-aggressive prostate cancer.

Quantitative mass spectrometry data of glycopeptides from a discovery cohort comprised of 74 aggressive (Gleason score≥8) and 68 non-aggressive (Gleason score=6) prostate cancer urine specimens were acquired via a data independent acquisition approach. The glycopeptides showing distinct expression profiles in aggressive relative to non-aggressive prostate cancer were further evaluated for their performance in distinguishing the two groups either individually or in combination with others using repeated 5-fold cross validation with logistic regression to build predictive models. Predictive models showing good performance from the discovery cohort were further evaluated using a validation cohort.

Among the 20 candidate glycoproteins, urinary ACPP outperformed the other candidates. Urinary ACPP can also serve as an adjunct to serum PSA to further improve the discrimination power for aggressive prostate cancer (AUC=0.82, 95% confidence interval 0.75 to 0.89). A three-signature panel including urinary ACPP, urinary CLU, and serum PSA displayed the ability to distinguish aggressive prostate cancer from non-aggressive prostate cancer with an AUC of 0.86 (95% confidence interval 0.8 to 0.92). Another three-signature panel containing urinary ACPP, urinary LOX, and serum PSA also demonstrated its ability in recognizing aggressive prostate cancer (AUC=0.82, 95% confidence interval 0.75 to 0.9). Moreover, consistent performance was observed from each panel when evaluated using a validation cohort.

The present inventors have identified glycopeptides of urinary glycoproteins associated with aggressive prostate cancer using a quantitative mass spectrometry-based glycoproteomic approach and demonstrated their potential to serve as noninvasive urinary glycoprotein biomarkers worthy of further validation by a multi-center study.

### I. Definitions

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, wood chucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets. In various embodiments, the subject is mouse or mice. In various embodiments, the subject, patient or individual is human.

A subject can be one who has been previously diagnosed with or identified as suffering from or having a condition, disease, or disorder in need of treatment (e.g., prostate cancer) or one or more complications related to the condition, disease, or disorder, and optionally, have already undergone treatment for the condition, disease, disorder, or the one or more complications related to the condition, disease, or disorder. Alternatively, a subject can also be one who has not been previously diagnosed as having a condition, disease, or disorder or one or more complications related to the condition, disease, or disorder. For example, a subject can be one who exhibits one or more risk factors for a condition, disease, or disorder, or one or more complications related to the condition, disease, or disorder, or a subject who does not exhibit risk factors. A “subject in need” of treatment for a particular condition, disease, or disorder can be a subject suspected of having that condition, disease, or disorder, diagnosed as having that condition, disease, or disorder, already treated or being treated for that condition, disease, or disorder, not treated for that condition, disease, or disorder, or at risk of developing that condition, disease, or disorder.

In some embodiments, the subject is selected from the group consisting of a subject suspected of having a disease, a subject that has a disease, a subject diagnosed with a disease, a subject that has been treated for a disease, a subject that is being treated for a disease, and a subject that is at risk of developing a disease.

In some embodiments, the subject is selected from the group consisting of a subject suspected of having prostate cancer, a subject that has prostate cancer, a subject diagnosed with prostate cancer, a subject that has non-aggressive prostate cancer, a subject suspected of having aggressive prostate cancer, a subject that has been treated for prostate cancer, a subject that is being treated for prostate cancer, and a subject that is at risk of developing prostate cancer.

By “at risk of” is intended to mean at increased risk of, compared to a normal subject, or compared to a control group, e.g., a patient population. Thus, a subject carrying a particular marker may have an increased risk for a specific condition, disease or disorder, and be identified as needing further testing. “Increased risk” or “elevated risk” mean any statistically significant increase in the probability, e.g., that the subject has the disorder. The risk is increased by at least 10%, at least 20%, and even at least 50% over the control group with which the comparison is being made. In certain embodiments, a subject can be at risk of developing aggressive prostate cancer.

“Sample” is used herein in its broadest sense. The term “biological sample” as used herein denotes a sample taken or isolated from a biological organism. A sample or biological sample may comprise a bodily fluid including blood, serum, plasma, tears, aqueous and vitreous humor, spinal fluid; a soluble fraction of a cell or tissue preparation, or media in which cells were grown; or membrane isolated or extracted from a cell or tissue; polypeptides, or peptides in solution or bound to a substrate; a cell; a tissue, a tissue print, a fingerprint, skin or hair; fragments and derivatives thereof. Non-limiting examples of samples or biological samples include cheek swab; mucus; whole blood, blood, serum; plasma; urine; saliva, semen; lymph; fecal extract; sputum; other body fluid or biofluid; cell sample; and tissue sample etc. The term also includes a mixture of the above-mentioned samples or biological samples. The term “sample” also includes untreated or pretreated (or pre-processed) biological samples. In some embodiments, a sample or biological sample can comprise one or more cells from the subject. Subject samples or biological samples usually comprise derivatives of blood products, including blood, plasma and serum. In some embodiments, the sample is a biological sample. In some embodiments, the sample is blood. In some embodiments, the sample is plasma. In some embodiments, the sample is blood, plasma, serum, or urine. In certain embodiments, the sample is a serum sample. In particular embodiments, the sample is a urine sample.

The terms “body fluid” or “bodily fluids” are liquids originating from inside the bodies of organisms. Bodily fluids include amniotic fluid, aqueous humour, vitreous humour, bile, blood (e.g., serum), breast milk, cerebrospinal fluid, cerumen (earwax), chyle, chyme, endolymph and perilymph, exudates, feces, female ejaculate, gastric acid, gastric juice, lymph, mucus (e.g., nasal drainage and phlegm), pericardial fluid, peritoneal fluid, pleural fluid, pus, rheum, saliva, sebum (skin oil), serous fluid, semen, sputum, synovial fluid, sweat, tears, urine, vaginal secretion, and vomit. Extracellular bodily fluids include intravascular fluid (blood plasma), interstitial fluids, lymphatic fluid and transcellular fluid. “Biological sample” also includes a mixture of the above-mentioned body fluids. “Biological samples” may be untreated or pretreated (or pre-processed) biological samples.

Sample collection procedures and devices known in the art are suitable for use with various embodiment of the present invention. Examples of sample collection procedures and devices include but are not limited to: phlebotomy tubes (e.g., a vacutainer blood/specimen collection device for collection and/or storage of the blood/specimen), dried blood spots, Microvette CB300 Capillary Collection Device (Sarstedt), HemaXis blood collection devices (microfluidic technology, Hemaxis), Volumetric Absorptive Microsampling (such as CE-IVD

Mitra microsampling device for accurate dried blood sampling (Neoteryx), HemaSpot™-HF Blood Collection Device, a tissue sample collection device; standard collection/storage device (e.g., a collection/storage device for collection and/or storage of a sample (e.g., blood, plasma, serum, urine, etc.); a dried blood spot sampling device. In some embodiments, the Volumetric Absorptive Microsampling (VAMS^{1M}) samples can be stored and mailed, and an assay can be performed remotely.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, -carboxyglutamate, and O-phosphoserine. Amino acid analogs refers to compounds that have the same basic chemical structure as a naturally occurring amino acid, i.e., an carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function s in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “peptide” as used herein refers to any compound containing at least two amino acid residues joined by an amide bond formed from the carboxyl group of one amino acid residue and the amino group of the adjacent amino acid residue. In some embodiments, peptide refers to a polymer of amino acid residues typically ranging in length from 2 to about 30, or to about 40, or to about 50, or to about 60, or to about 70 residues. In certain embodiments the peptide ranges in length from about 2, 3, 4, 5, 7, 9, 10, or 11 residues to about 60, 50, 45, 40, 45, 30, 25, 20, or 15 residues. In certain embodiments the peptide ranges in length from about 8, 9, 10, 11, or 12 residues to about 15, 20 or 25 residues. In some embodiments, the peptide ranges in length from 2 to about 12 residues, or 2 to about 20 residues, or 2 to about 30 residues, or 2 to about 40 residues, or 2 to about 50 residues, or 2 to about 60 residues, or 2 to about 70 residues. In certain embodiments the amino acid residues comprising the peptide are “L-form” amino acid residues, however, it is recognized that in various embodiments, “D” amino acids can be incorporated into the peptide. Peptides also include amino acid polymers in which one or more amino acid residues are an artificial chemical analogue of a corresponding naturally occurring amino acid, as well as to naturally occurring amino acid polymers. In addition, the term applies to amino acids joined by a peptide linkage or by other, “modified linkages” (e.g., where the peptide bond is replaced by an a-ester, a f3-ester, a thioamide, phosphonamide, carbamate, hydroxylate, and the like (see, e.g., Spatola, (1983) Chern. Biochem. Amino Acids and Proteins 7: 267-357), where the amide is replaced with a saturated amine (see, e.g., Skiles et al., U.S. Pat. No. 4,496,542, which is incorporated herein by reference, and Kaltenbronn et al., (1990) pp. 969-970 in Proc. 'I 1th American Peptide Symposium, ESCOM Science Publishers, The Netherlands, and the like)).

A protein refers to any of a class of nitrogenous organic compounds that comprise large molecules composed of one or more long chains of amino acids and are an essential part of all living organisms. A protein may contain various modifications to the amino acid structure such as disulfide bond formation, phosphorylations and glycosylations. A linear chain of amino acid residues may be called a “polypeptide,” A protein contains at least one polypeptide. Short polypeptides, e.g., containing less than 20-30 residues, are sometimes referred to as “peptides.”

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy-chain variable region.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity).

The term “threshold” as used herein refers to the magnitude or intensity that must be exceeded for a certain reaction, phenomenon, result, or condition to occur or be considered relevant. The relevance can depend on context, e.g., it may refer to a positive, reactive or statistically significant relevance.

By “binding assay” is meant a biochemical assay wherein the biomarkers are detected by binding to an agent, such as an antibody, through which the detection process is carried out. The detection process may involve fluorescent or radioactive labels, and the like. The assay may involve immobilization of the biomarker, or may take place in solution.

“Immunoassay” is an assay that uses an antibody to specifically bind an antigen (e.g., a marker). The immunoassay is characterized by the use of specific binding properties of a particular antibody to isolate, target, and/or quantify the antigen. Non-limiting examples of immunoassays include ELISA (enzyme-linked immunosorbent assay), immunoprecipitation, SISCAPA (stable isotope standards and capture by anti-peptide antibodies), Western blot, etc.

“Diagnostic” means identifying the presence or nature of a pathologic condition, disease, or disorder and includes identifying patients who are at risk of developing a specific condition, disease or disorder. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, a disease, or a disorder, it suffices if the method provides a positive indication that aids in diagnosis.

The term “statistically significant” or “significantly” refers to statistical evidence that there is a difference. It is defined as the probability of making a decision to reject the null hypothesis when the null hypothesis is actually true. The decision is often made using the p-value.

The terms “detection”, “detecting” and the like, may be used in the context of detecting biomarkers, detecting peptides, detecting proteins, or of detecting a condition, detecting a disease or a disorder (e.g., when positive assay results are obtained). In the latter context, “detecting” and “diagnosing” are considered synonymous when mere detection indicates the diagnosis.

The terms “marker” or “biomarker” are used interchangeably herein, and in the context of the present invention refer to a protein or peptide (for example, protein or peptide associated with prostate cancer or prostate cancer as described herein) is differentially present in a sample taken from patients having a specific disease or disorder as compared to a control value, the control value consisting of, for example average or mean values in comparable samples taken from control subjects (e.g., a person with a negative diagnosis, normal or healthy subject). Biomarkers may be determined as specific peptides or proteins which may be detected by, for example, antibodies or mass spectroscopy. In some applications, for example, a mass spectroscopy or other profile of multiple antibodies may be used to determine multiple biomarkers, and differences between individual biomarkers and/or the partial or complete profile may be used for diagnosis. In some embodiments, the biomarkers may be detected by antibodies, mass spectrometry, or combinations thereof.

A “test amount” of a marker refers to an amount of a marker present in a sample being tested. A test amount can be either in absolute amount (e.g., g/mi) or a relative amount (e.g., relative intensity of signals).

A “diagnostic amount” of a marker refers to an amount of a marker in a subject's sample that is consistent with a diagnosis of a particular disease or disorder. A diagnostic amount can be either in absolute amount (e.g., μg/ml) or a relative amount (e.g., relative intensity of signals).

A “control amount” of a marker can be any amount or a range of amount which is to be compared against a test amount of a marker. For example, a control amount of a marker can be the amount of a marker in a person who does not suffer from the disease or disorder sought to be diagnosed, A control amount can be either in absolute amount (e.g., μg/ml) or a relative amount (e.g., relative intensity of signals).

The term “differentially present” or “change in level” refers to differences in the quantity and/or the frequency of a marker present in a sample taken from patients having a specific disease or disorder as compared to a control subject. For example, a marker can be present at an elevated level or at a decreased level in samples of patients with the disease or disorder compared to a control value (e.g., determined from samples of control subjects). Alternatively, a marker can be detected at a higher frequency or at a lower frequency in samples of patients compared to samples of control subjects. A marker can be differentially present in terms of quantity, frequency or both as well as a ratio of differences between two or more specific modified amino acid residues and/or the protein itself. In one embodiment, an increase in the ratio of modified to unmodified proteins and peptides described herein is diagnostic of any one or more of the diseases described herein. In particular embodiments, a marker can be differentially present in patients having aggressive prostate cancer as compared to a control subject including patients having non-aggressive prostate cancer or no cancer.

A marker, compound, composition or substance is differentially present in a sample if the amount of the marker, compound, composition or substance in the sample (a patient having aggressive prostate cancer) is statistically significantly different from the amount of the marker, compound, composition or substance in another sample (a patient having non-aggressive cancer or no cancer), or from a control value (e.g., an index or value representative of non-aggressive cancer or no cancer). For example, a compound is differentially present if it is present at least about 120%, at least about 130%, at least about 150%, at least about 180%, at least about 200%, at least about 300%, at least about 500%, at least about 700%, at least about 900%, or at least about 1000% greater or less than it is present in the other sample (e.g., control), or if it is detectable in one sample and not detectable in the other.

Alternatively, or additionally, a marker, compound, composition or substance is differentially present between samples if the frequency of detecting the marker, etc. in samples of patients suffering from a particular disease or disorder, is statistically significantly higher or lower than in the control samples or control values obtained from controls such as a subject having non-aggressive prostate cancer, benign lesions and the like, or otherwise healthy individuals. For example, a biomarker is differentially present between the two sets of samples if it is detected at least about 10%, at least about 20%, at least about 30%, at least about 40%, at least about 50%, at least about 60%, at least about 70%, at least about 80%, at least about 90%, or at least about 100% more frequently or less frequently observed in one set of samples (e.g., a patient having aggressive prostate cancer) than the other set of samples (e.g., a patient having non-aggressive prostate cancer or no cancer). These exemplary values notwithstanding, it is expected that a skilled practitioner can determine cut-off points, etc., that represent a statistically significant difference to determine whether the marker is differentially present.

The term “one or more of” refers to combinations of various biomarkers. The term encompasses 1, 2, 3, 4, 5, 6, 7, 8, 9, 10, 11, 12, 13, 15 ,16 ,17, 18, 19, 20, 21, 22, 23, 24, 25, 26, 27, 28, 29, 30, 31, 32, 33, 34, 35, 36, 37, 38, 39, 40 . . . N, where “N” is the total number of biomarker proteins in the particular embodiment. The term also encompasses, and is interchangeably used with, at least 1, at least 2, at least 3, at least 4, at least 5, at least 6, at least 7, at least 8, at least 9, at least 10, at least 11, at least 12, at least 13, at least 15 ,16 ,17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40 . . . N. It is understood that the recitation of biomarkers herein includes the phrase “one or more of” the biomarkers and, in particular, includes the “at least 1, at least 2, at least 3” and so forth language in each recited embodiment of a biomarker panel.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, digoxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, flow cytometry, or direct analysis by mass spectrometry of intact protein or peptides. In some embodiments, the detectable moiety is a stable isotope. In some embodiments, the stable isotope is selected from the group consisting of ¹⁵N, ¹³C, ¹⁸O and ²H.

As used herein, the terms “treat”, “treatment”, “treating”, or “amelioration” when used in reference to a disease, disorder or medical condition, refer to both therapeutic treatment and prophylactic or preventative measures, wherein the object is to reverse, alleviate, ameliorate, inhibit, lessen, slow down or stop the progression or severity of a symptom, a condition, a disease, or a disorder. The term “treating” includes reducing or alleviating at least one adverse effect or symptom of a condition, a disease, or a disorder. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition, disease, or disorder even if the treatment is ultimately unsuccessful. Those in need of treatment include those already with the condition, disease, or disorder as well as those prone to have the condition, disease, or disorder or those in whom the condition, disease, or disorder is to be prevented.

Non-limiting examples of treatments or therapeutic treatments include pharmacological or biological therapies and/or interventional surgical treatments.

The term “preventative treatment” means maintaining or improving a healthy state or non-diseased state of a healthy subject or subject that does not have a disease. The term “preventative treatment” or “health surveillance” also means to prevent or to slow the appearance of symptoms associated with a condition, disease, or disorder. The term “preventative treatment” also means to prevent or slow a subject from obtaining a condition, disease, or disorder.

As used herein, the term “administering,” refers to the placement an agent or a treatment as disclosed herein into a subject by a method or route which results in at least partial localization of the agent or treatment at a desired site. “Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrastemai, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. In accordance with the present invention, “administering” can be self-administering. For example, it is considered as “administering” that a subject consumes a composition as disclosed herein.

### II. Measurement/Detection of Markers

In one aspect, the present invention provides compositions and methods for measuring one or more proteins. In certain embodiments, the one or more proteins are glycosylated proteins/peptides. In specific embodiments, the glycosylated proteins/peptides comprises one or more of ACPP, CD63, KLK 11, ATRN, GP2, PTPRN2, NPTN, RNASE2, CPE, SERPINA1, DSC2, PTGDS, GRN, LRG1, UMOD, CLU, LOX, ORM1, CD97, and AFM. In other embodiments, the present invention also comprises measurement of serum PSA. In alternative embodiments, the present invention utilizes serum PSA in the detection of aggressive prostate cancer.

In any of the embodiments recited herein, the biomarker glycoproteins of interest comprise ACPP and one or more of CD63, KLK 11, ATRN, GP2, PTPRN2, NPTN, RNASE2, CPE, SERPINA1, DSC2, PTGDS, GRN, LRG1, UMOD, CLU, LOX, ORM1, CD97, and AFM. In yet another embodiment, the biomarker glycoproteins of interest comprise ACPP and one or more of DSC2, PTGDS, GRN, CD97, AFM, LRG1, and UMOD. In a further embodiment, the biomarker glycoproteins of interest comprise ACPP and one or more of SERPINA1, CLU, LOX, and ORM1. In certain embodiments, the biomarker glycoproteins of interest comprise one or more of CD63, KLK 11, ATRN, GP2, PTPRN2, NPTN, RNASE2, CPE and one or more of SERPINA1, DSC2, PTGDS, GRN, LRG1, UMOD, CLU, LOX, ORM1, CD97, and AFM.

In one embodiment, a panel of biomarkers comprises urine ACPP and serum PSA. In another embodiment, a panel of biomarkers comprises urine ACPP and urine CLU. In yet another embodiment, a panel of biomarkers comprises urine ACPP and urine LOX. In a specific embodiment, a panel of biomarkers comprises urine ACPP and urine SERPINA1. In another specific embodiment, a panel of biomarkers comprises urine ACPP and urine ORM1.

In one embodiment, a panel of biomarkers comprises urine ACPP, urine CLU and serum PSA. In another embodiment, a panel of biomarkers comprises ACPP, urine LOX and serum PSA. In a specific embodiment, a panel of biomarkers comprises urine ACPP, urine SERPINA1 and serum PSA. In another specific embodiment, a panel of biomarkers comprises urineACPP, urine ORM1 and serum PSA.

In another aspect, the measured proteins can be used further to determine certain aspects associated with prostate cancer. For example, the measured proteins can be used to identify a subject as having prostate cancer. In certain embodiments, the proteins can be used to assess prostate cancer severity (e.g., aggressive vs. non-aggressive), predict survival, and predict response to therapy.

### A. Measurement/Detection by Mass Spectrometry

In various embodiments the invention provides a method to identify protein biomarkers and patterns that are indicative of a disease. In various embodiments the invention provides a method to identify protein biomarkers and patterns that are indicative a disease is or may be present. In some embodiments these methods may provide objective rationale for further testing. In various embodiments the invention provides a method for the identification of a plurality of proteins from a sample, wherein each protein is correlated to one or more peptides, wherein each peptide is correlated to one or more transitions, wherein each transition comprises a Q1 mass value. In various embodiments the invention provides a method for the identification of a plurality of proteins from a sample, wherein each protein is correlated to one or more peptides, wherein each peptide is correlated to one or more transitions, wherein each transition comprises a Q1 mass value and a Q3 mass value. In various embodiments the invention provides a method for the identification of a plurality of proteins from a sample, wherein each protein is correlated to one or more peptides, wherein each peptide is correlated to one or more transitions, wherein each transition comprises a Q1/Q3 mass value pair.

As used herein, SRM stands for selected reaction monitoring. As used herein, MRM stands for multiple reaction monitoring. As used herein, PRM stands for parallel reaction monitoring. As used herein, SWATH stands for sequential window acquisition of all theoretical fragment ion spectra. As used herein, DIA stands for data-independent acquisition. As used herein, MS stands for mass spectrometry. As used herein, SIL stands for stable isotope-labeled.

As used herein, “MS data” can be raw MS data obtained from a mass spectrometer and/or processed MS data in which peptides and their fragments (e.g., transitions and MS peaks) are already identified, analyzed and/or quantified. MS data can be Selective Reaction Monitoring (SRM) data, Multiple Reaction Monitoring (MRM) data, parallel reaction monitoring (PRM) data, Shotgun CID MS data, Original DIA MS Data, MSE MS data, p2CID MS Data, PAcIFIC MS Data, AIF MS Data, XDLA MS Data, SWATH MS data, or FT-ARM MS Data, or their combinations.

In some embodiments of the present invention, based on SRM and/or MS, and/or PRM MS, allows for the detection and accurate quantification of specific peptides in complex mixtures.

Selected Reaction Monitoring or Multiple Reaction Monitoring (SRM/MRM) mass spectrometry is a technology with the potential for reliable and comprehensive quantification of substances of low abundance in complex samples. SRM is performed on triple quadrupole-like instruments, in which increased selectivity is obtained through collision-induced dissociation. It is a non-scanning mass spectrometry technique, where two mass analyzers (Q1 and Q3) are used as static mass filters, to monitor a particular fragment of a selected precursor. On triple quadrapole instruments, various ionization methods can be used including without limitation electrospray ionization, chemical ionization, electron ionization, atmospheric pressure chemical ionization, and matrix-assisted laser desorption ionization. Both the first mass analyzer and the collision cell are continuously exposed to ions from the source in a time dependent manner. Once the ions move into the third mass analyzer time dependence becomes a factor. On triple quadrupole instruments, the first quadrapole mass filter, Q1 is the primary m/z selector after the sample leaves the ionization source. Any ions with mass-to-charge ratios other than the one selected for will not be allowed to infiltrate Q1. The collision cell, denoted as “q2”, located between the first quadrapole mass filter Q1 and second quadrapole mass filter Q3, is where fragmentation of the sample occurs in the presence of an inert gas like argon, helium, or nitrogen. Upon exiting the collision cell, the fragmented ions then travel onto the second quadrapole mass filter Q3, where m/z selection can occur again. The specific pair of mass-over-charge (m/z) values associated to the precursor and fragment ions selected is referred to as a “transition”. The detector acts as a counting device for the ions matching the selected transition thereby returning an intensity distribution over time. MRM is when multiple SRM transitions are measured within the same experiment on the chromatographic time scale by rapidly switching between the different precursor/fragment pairs. Typically, the triple quadrupole instrument cycles through a series of transitions and records the signal of each transition as a function of the elution time. The method allows for additional selectivity by monitoring the chromatographic co-elution of multiple transitions for a given analyte.

In addition to MRM, the choice of peptides can also be quantified through Parallel-Reaction Monitoring (PRM), Parallel reaction monitoring (PRM) is the application of SRM with parallel detection of all transitions in a single analysis using a high resolution mass spectrometer. PRM provides high selectivity, high sensitivity and high-throughput to quantify selected peptide (Q1), hence quantify proteins. Again, multiple peptides can be specifically selected for each protein. PRM methodology uses the quadrupole of a mass spectrometer to isolate a target precursor ion, fragments the targeted precursor ion in the collision cell, and then detects the resulting product ions in the Orbitrap mass analyzer. Quantification is carried out after data acquisition by extracting one or more fragment ions with 5-10 ppm mass windows. PRM uses a quadrupole time-of-flight (QTOF) or hybrid quadrupole-orbitrap (QOrbitrap) mass spectrometer to carry out the peptides/ proteins quantitation. Examples of QTOF include but are not limited to: TripleTOF® 6600 or 5600 System (Sciex); X500R QTOF System (Sciex); 6500 Series Accurate-Mass Quadrupole Time-of-Flight (Q-TOF) (Agilent); or Xevo G2-XS QTof Quadrupole Time-of-Flight Mass Spectrometry (Waters). Examples of QObitrap include but are not limited to: Q Exactive™ Hybrid Quadrupole-Orbitrap Mass Spectrometer (the Thermo Scientific); or Orbitrap Fusion™ Tribrid™ (the Thermo Scientific).

Non-limiting advantages of PRM include elimination of most interferences, provides more accuracy and attomole-level limits of detection and quantification, enables the confident confirmation of the peptide identity with spectral library matching, reduces assay development time since no target transitions need to be preselected, ensures UHPLC-compatible data acquisition speeds with spectrum multiplexing and advanced signal processing.

SWATH MS is a data independent acquisition (DIA) method which aims to complement traditional mass spectrometry-based proteomics techniques such as shotgun and SRM methods. In essence, it allows a complete and permanent recording of all fragment ions of the detectable peptide precursors present in a biological sample. It thus combines the advantages of shotgun (high throughput) with those of SRM (high reproducibility and consistency).

In some embodiments, the developed methods herein can be applied to the quantification of polypeptides(s) or protein(s) in biological sample(s), such as urine and/or serum. Any kind of biological samples comprising polypeptides or proteins can be the starting point and be analyzed by the methods herein. Indeed, any protein/peptide containing sample can be used for and analyzed by the methods produced here (e.g., tissues, cells). The methods herein can also be used with peptide mixtures obtained by digestion. Digestion of a polypeptide or protein includes any-kind of cleavage strategies such as enzymatic, chemical, physical or combinations thereof.

In some embodiments, the analysis and/or comparison is performed on protein samples of wild-type or physiological/healthy origin against protein samples of mutant or pathological origin.

### B. Measurement/Detection by Immunoassays

In specific embodiments, the proteins of the present invention can be detected and/or measured by immunoassay. Immunoassay requires biospecific capture reagents/binding agent, such as antibodies, to capture the biomarkers. Many antibodies are available commercially. Antibodies also can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well-known in the art. Biospecific capture reagents useful in an immunoassay can also include lectins. The biospecific capture reagents can, in some embodiments, bind all forms of the biomarker, e.g., PSA and its post-translationally modified forms (e.g., glycosylated form). In other embodiments, the biospecific capture reagents bind the specific biomarker and not similar forms thereof.

The present invention contemplates traditional immunoassays including, for example, sandwich immunoassays including ELISA or fluorescence-based immunoassays, immunoblots, Western Blots (WB), as well as other enzyme immunoassays. Nephelometry is an assay performed in liquid phase, in which antibodies are in solution. Binding of the antigen to the antibody results in changes in absorbance, which is measured. In a SELDI-based immunoassay, a biospecific capture reagent for the biomarker is attached to the surface of an MS probe, such as a pre-activated protein chip array. The biomarker is then specifically captured on the biochip through this reagent, and the captured biomarker is detected by mass spectrometry.

In certain embodiments, the expression levels of the protein biomarkers employed herein are quantified by immunoassay, such as enzyme-linked immunoassay (ELISA) technology. In specific embodiments, the levels of expression of the biomarkers are determined by contacting the biological sample with antibodies, or antigen binding fragments thereof, that selectively bind to the biomarker; and detecting binding of the antibodies, or antigen binding fragments thereof, to the biomarkers. In certain embodiments, the binding agents employed in the disclosed methods and compositions are labeled with a detectable moiety. In other embodiments, a binding agent and a detection agent are used, in which the detection agent is labeled with a detectable moiety. For ease of reference, the term antibody is used in describing binding agents or capture molecules. However, it is understood that reference to an antibody in the context of describing an exemplary binding agent in the methods of the present invention also includes reference to other binding agents including, but not limited to lectins.

For example, the level of a biomarker in a sample can be assayed by contacting the biological sample with an antibody, or antigen binding fragment thereof, that selectively binds to the target protein (referred to as a capture molecule or antibody or a binding agent), and detecting the binding of the antibody, or antigen-binding fragment thereof, to the protein. The detection can be performed using a second antibody to bind to the capture antibody complexed with its target biomarker. A target biomarker can be an entire protein, or a variant or modified form thereof. Kits for the detection of proteins as described herein can include pre-coated strip/plates, biotinylated secondary antibody, standards, controls, buffers, streptavidin-horse radish peroxidise (HRP), tetramethyl benzidine (TMB), stop reagents, and detailed instructions for carrying out the tests including performing standards.

The present disclosure also provides methods for detecting protein in a sample obtained from a subject, wherein the levels of expression of the proteins in a biological sample are determined simultaneously. For example, in one embodiment, methods are provided that comprise: (a) contacting a biological sample obtained from the subject with a plurality of binding agents that each selectively bind to one or more biomarker proteins for a period of time sufficient to form binding agent-biomarker complexes; and (b) detecting binding of the binding agents to the one or more biomarker proteins. In further embodiments, detection thereby determines the levels of expression of the biomarkers in the biological sample; and the method can further comprise (c) comparing the levels of expression of the one or more biomarker proteins in the biological sample with predetermined threshold values, wherein levels of expression of at least one of the biomarker proteins above or below the predetermined threshold values indicates, for example, the subject has prostate cancer, the severity of prostate cancer, and/or is/will be responsive to prostate cancer therapy. Examples of binding agents that can be effectively employed in such methods include, but are not limited to, antibodies or antigen-binding fragments thereof, aptamers, lectins and the like.

Although antibodies are useful because of their extensive characterization, any other suitable agent (e.g., a peptide, an aptamer, or a small organic molecule) that specifically binds a biomarker of the present invention is optionally used in place of the antibody in the above described immunoassays. For example, an aptamer that specifically binds a biomarker and/or one or more of its breakdown products might be used. Aptamers are nucleic acid-based molecules that bind specific ligands. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

In specific embodiments, the assay performed on the biological sample can comprise contacting the biological sample with one or more capture agents (e.g., antibodies, lectins, peptides, aptamer, etc., combinations thereof) to form a biomarker:capture agent complex. The complexes can then be detected and/or quantified. A subject can then be identified as having aggressive prostate cancer based on a comparison of the detected/quantified/measured levels of biomarkers to one or more reference controls as described herein. The biomarker levels can also be utilized with other biomarker measurements including, but not limited to serum PSA.

In one method, a first, or capture, binding agent, such as an antibody that specifically binds the protein biomarker of interest, is immobilized on a suitable solid phase substrate or carrier. The test biological sample is then contacted with the capture antibody and incubated for a desired period of time. After washing to remove unbound material, a second, detection, antibody that binds to a different, non-overlapping, epitope on the biomarker (or to the bound capture antibody) is then used to detect binding of the polypeptide biomarker to the capture antibody. The detection antibody is preferably conjugated, either directly or indirectly, to a detectable moiety. Examples of detectable moieties that can be employed in such methods include, but are not limited to, cheminescent and luminescent agents; fluorophores such as fluorescein, rhodamine and eosin; radioisotopes; colorimetric agents; and enzyme-substrate labels, such as biotin.

In a more specific embodiment, a biotinylated lectin that specifically binds a biomarker (e.g., ACPP) can be added to a patient sample and a streptavidin labeled fluorescent marker that binds the biotinylated lectin bound to the biomarker is then added, and the biomarker is detected.

In another embodiment, the assay is a competitive binding assay, wherein labeled protein biomarker is used in place of the labeled detection antibody, and the labeled biomarker and any unlabeled biomarker present in the test sample compete for binding to the capture antibody. The amount of biomarker bound to the capture antibody can be determined based on the proportion of labeled biomarker detected.

Solid phase substrates, or carriers, that can be effectively employed in such assays are well known to those of skill in the art and include, for example, 96 well microtiter plates, glass, paper, and microporous membranes constructed, for example, of nitrocellulose, nylon, polyvinylidene difluoride, polyester, cellulose acetate, mixed cellulose esters and polycarbonate. Suitable microporous membranes include, for example, those described in US Patent Application Publication no. US 2010/0093557 A1. Methods for the automation of immunoassays are well known in the art and include, for example, those described in U.S. Pat. Nos. 5,885,530, 4,981,785, 6,159,750 and 5,358,691.

The presence of several different protein biomarkers in a test sample can be detected simultaneously using a multiplex assay, such as a multiplex ELISA. Multiplex assays offer the advantages of high throughput, a small volume of sample being required, and the ability to detect different proteins across a board dynamic range of concentrations.

In certain embodiments, such methods employ an array, wherein multiple binding agents (for example capture antibodies) specific for multiple biomarkers are immobilized on a substrate, such as a membrane, with each capture agent being positioned at a specific, pre-determined, location on the substrate. Methods for performing assays employing such arrays include those described, for example, in US Patent Application Publication nos. US2010/0093557A1 and US2010/0190656A1, the disclosures of which are hereby specifically incorporated by reference.

Multiplex arrays in several different formats based on the utilization of, for example, flow cytometry, chemiluminescence or electron-chemiluminesence technology, can be used. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-biomarker complexes formed on the bead set. Multiple biomarkers can be recognized and measured by differences in the bead sets, with chromogenic or fluorogenic emissions being detected using flow cytometric analysis.

In an alternative format, a multiplex ELISA from Quansys Biosciences (Logan, Utah) coats multiple specific capture antibodies at multiple spots (one antibody at one spot) in the same well on a 96-well microtiter plate. Chemiluminescence technology is then used to detect multiple biomarkers at the corresponding spots on the plate.

In several embodiments, the biomarkers of the present invention may be detected by means of an electrochemicaluminescent assay developed by Meso Scale Discovery (Gaithersrburg, MD). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ˜620 nm, eliminating problems with color quenching. See U.S. Pat. Nos. 7,497,997; 7,491,540; 7,288,410; 7,036,946; 7,052,861; 6,977,722; 6,919,173; 6,673,533; 6,413,783; 6,362,011; 6,319,670; 6,207,369; 6,140,045; 6,090,545; and 5,866,434. See also U.S. Patent Applications Publication No. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534.

### C. Measurement/Detection By Other Detection Methods

The proteins of the present invention can be detected by other suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In particular embodiments, the protein biomarker proteins of the present invention can be captured and concentrated using nano particles. In a specific embodiment, the proteins can be captured and concentrated using Nanotrap® technology (Ceres Nanosciences, Inc. (Manassas, VA)). Briefly, the Nanotrap platform reduces pre-analytical variability by enabling biomarker enrichment, removal of high-abundance analytes, and by preventing degradation to highly labile analytes in an innovative, one-step collection workflow. Multiple analytes sequestered from a single sample can be concentrated and eluted into small volumes to effectively amplify, up to 100-fold or greater depending on the starting sample volume (Shafagati, 2014; Shafagati, 2013; Longo, et al., 2009), resulting in substantial improvements to downstream analytical sensitivity.

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, CA.), Invitrogen Corp. (Carlsbad, CA), Affymetrix, Inc. (Fremong, CA), Zyomyx (Hayward, CA), R&D Systems, Inc. (Minneapolis, MN), Biacore (Uppsala, Sweden) and Procognia (Berkshire, UK). Examples of such protein biochips are described in the following patents or published patent applications: U.S. Pat. Nos. 6,537,749; 6,329,209; 6,225,047; 5,242,828; PCT International Publication No. WO 00/56934; and PCT International Publication No. WO 03/048768.

In a particular embodiment, the present invention comprises a microarray chip. More specifically, the chip comprises a small wafer that carries a collection of binding agents bound to its surface in an orderly pattern, each binding agent occupying a specific position on the chip. The set of binding agents specifically bind to each of the one or more one or more of the biomarkers described herein. In particular embodiments, a few micro-liters of blood serum or plasma are dropped on the chip array. Protein biomarkers present in the tested specimen bind to the binding agents specifically recognized by them. Subtype and amount of bound mark is detected and quantified using, for example, a fluorescently-labeled secondary, subtype-specific antibody. In particular embodiments, an optical reader is used for bound biomarker detection and quantification. Thus, a system can comprise a chip array and an optical reader. In other embodiments, a chip is provided.

### III. Treatment Methods

In another aspect, the present invention provides a prostate cancer therapy or therapeutic interventions practically applied following the measurement/detection of biomarker glycopeptides. In particular embodiments, therapeutic intervention comprises prostatectomy, radiation therapy, cryotherapy (also referred to as cryosurgery or cryoablation), hormone therapy, chemotherapy, immunotherapy and combinations thereof.

Prostatectomy includes radical prostatectomy (open (radical retropubic prostatectomy or radical perineal prostatectomy) or lateral (laparoscopic radical prostatectomy including robotic-assisted), and transurethral resection of the prostate (TURP).

Radiation therapy includes external beam radiation (three-dimensional conformal radiation therapy (3D-CRT), intensity modulated radiation therapy (IMIRT), stereotactic body radiation therapy (SBRT), proton beam radiation therapy) and brachytherapy (internal radiation) (permanent (low dose rate or LDR) brachytherapy or temporary (high dose rate or HDR) brachytherapy).

Hormone therapy (androgen suppression therapy) includes orchiectomy (surgical castration), luteinizing hormone-release hormone (LHRH) agonists (e.g., leuprolide, goserelin, triptorelin, histrelin), LHRH antagonists (e.g., degareli), treatment to lower androgen levels from the adrenal glands (e.g., abiraterone, ketoconazole), anti-androgens (e.g., flutamide, bicalutamide, nilutamide, enzalutamide, apalutamide), and estrogens.

Chemotherapy includes treatment with compounds including, but not limited to, docetaxel, cabazitaxel, mitoxantrone, and estramustine.

Immunotherapy includes, but is not limited to, a cancer vaccine (e.g., sipuleucel-T), as well as immune checkpoint inhibitors (e.g., PD-1 inhibitors including pembrolizumab). Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor).

A prostate therapeutic intervention can comprise a targeted therapy including poly(ADP)-ribose polymerase (PARP) inhibitor (e.g., niraparib (zejula), olaparib (lynparza), and rucaparib (rubraca)).

Other therapeutic interventions for prostate cancer include an androgen receptor (AR)-targeted therapy (e.g., enzalutamide, ARN-509, ODM-201, EPI-001, hydrazinobenzoylcurcumin (HBC), aberaterone, geleterone, and seviteronel), an antimicrotubule agent, an alkylating agent and an anthracenedione.

In particular embodiments, a therapeutic intervention for prostate cancer can include the administration of drugs including, but not limited to, Abiraterone Acetate, Apalutamide, Bicalutamide, Cabazitaxel, Casodex (Bicalutamide), Darolutamide, Degarelix, Docetaxel, Eligard (Leuprolide Acetate), Enzalutamide, Erleada (Apalutamide), Firmagon (Degarelix), Flutamide, Goserelin Acetate, Jevtana (Cabazitaxel), Leuprolide Acetate, Lupron (Leuprolide Acetate), Lupron Depot (Leuprolide Acetate), Lynparza (Olaparib), Mitoxantrone Hydrochloride, Nilandron (Nilutamide), Nilutamide, Nubeqa (Darolutamide), Olaparib, Provenge (Sipuleucel-T), Radium 223 Dichloride, Rubraca (Rucaparib Camsylate), Rucaparib Camsylate, Sipuleucel-T, Taxotere (Docetaxel), Xofigo (Radium 223 Dichloride), Xtandi (Enzalutamide), Zoladex (Goserelin Acetate), Zytiga (Abiraterone Acetate).

### IV. Kits

In another aspect, the present invention provides kits for detecting one or more biomarker proteins. The exact nature of the components configured in the inventive kit depends on its intended purpose. In one embodiment, the kit is configured particularly for human subjects.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

In various embodiments, the present invention provides a kit comprising: (a) one or more internal standards suitable for measurement of one or more proteins including any one or more of mass spectrometry, antibody method, antibodies, lectins, nucleic acid aptamer method, nucleic acid aptamers, immunoassay, ELISA, immunoprecipitation, SISCAPA, Western blot, or combinations thereof; and (b) reagents and instructions for sample processing, preparation and biomarker protein measurement/detection. The kit can further comprise (c) instructions for using the kit to measure biomarker proteins in a sample obtained from the subject.

In particular embodiments, the kit comprises reagents necessary for processing of samples and performance of an immunoassay. In a specific embodiment, the immunoassay is an ELISA. Thus, in certain embodiments, the kit comprises a substrate for performing the assay (e.g., a 96-well polystyrene plate). The substrate can be coated with antibodies specific for a biomarker protein. In a further embodiment, the kit can comprise a detection antibody including, for example, a polyclonal antibody specific for a biomarker protein conjugated to a detectable moiety or label (e.g., horseradish peroxidase). The kit can also comprise a standard, e.g., a human protein standard. The kit can also comprise one or more of a buffer diluent, calibrator diluent, wash buffer concentrate, color reagent, stop solution and plate sealers (e.g., adhesive strip).

In particular embodiments, the kit may comprise a solid support, such as a chip, microtiter plate (e.g., a 96-well plate), bead, or resin having protein biomarker capture reagents attached thereon. The kit may further comprise a means for detecting the protein biomarkers, such as antibodies, and a secondary antibody-signal complex such as horseradish peroxidase (HRP)-conjugated goat anti-rabbit IgG antibody and tetramethyl benzidine (TMB) as a substrate for HRP. In other embodiments, the kit can comprise magnetic beads conjugated to the antibodies (or separate containers thereof for later conjugation). The kit can further comprise detection antibodies, for example, biotinylated antibodies or lectins that can be detected using, for example, streptavidin labeled fluorescent markers such as phycoerythrin. The kit can be configured to perform the assay in a singleplex or multiplex format.

The kit may be provided as an immuno-chromatography strip comprising a membrane on which the antibodies are immobilized, and a means for detecting, e.g., gold particle bound antibodies, where the membrane, includes NC membrane and PVDF membrane. The kit may comprise a plastic plate on which a sample application pad, gold particle bound antibodies temporally immobilized on a glass fiber filter, a nitrocellulose membrane on which antibody bands and a secondary antibody band are immobilized and an absorbent pad are positioned in a serial manner, so as to keep continuous capillary flow of the sample.

In a specific embodiment, a kit comprises (a) magnetic beads for conjugating to antibodies that specifically bind biomarker proteins of interest; (b) monoclonal antibodies that specifically bind the biomarker proteins of interest; (c) biotinylated immunoglobulin G detection antibodies; (d) biotinylated lectins that specifically bind the biomarker proteins of interest; and (e) streptavidin labeled fluorescent marker.

In certain embodiments, a subject can be diagnosed by adding a biological sample (e.g., blood) from the patient to the kit and detecting the relevant protein biomarkers conjugated with antibodies/lectins, specifically, by a method which comprises the steps of: (i) collecting urine from the patient; (ii) adding urine from patient to a diagnostic kit; and, (iii) detecting the protein biomarkers conjugated with antibodies/lectins. If the biomarkers are present in the sample, the antibodies/lectins will bind to the sample, or a portion thereof. In other kit and diagnostic embodiments, urine will not be collected from the patient (i.e., it is already collected). Urine or other samples can be collected from subject of varying ages. Indeed, in other embodiments, the sample may comprise a serum, plasma sweat, tissue, blood or a clinical sample.

The kit can also comprise a washing solution or instructions for making a washing solution, in which the combination of the capture reagents and the washing solution allows capture of the protein biomarkers on the solid support for subsequent detection by, e.g., antibodies/lectins or mass spectrometry. In a further embodiment, a kit can comprise instructions for suitable operational parameters in the form of a label or separate insert. For example, the instructions may inform a consumer about how to collect the sample, etc. In yet another embodiment, the kit can comprise one or more containers with protein biomarker samples, to be used as standard(s) for calibration or normalization. Detection of the markers described herein may be accomplished using a lateral flow assay.

In certain embodiments, the kit comprises reagents and components necessary for performing an electrochemiluminescent ELISA.

In another aspect the present invention provides kit. In particular embodiments, a kit comprises (a) monoclonal antibodies that each specifically bind a biomarker protein of interest; (b) biotinylated immunoglobulin G detection antibodies; (c) biotinylated lectins that specifically bind glycosylated forms of the biomarker protein of interest; and (d) streptavidin labeled fluorescent markers. In a further embodiment, the kit further comprises (e) magnetic beads for conjugating to monoclonal antibodies that each specifically bind a biomarker protein of interest. In a specific embodiment, the biomarker protein of interest comprises one or more of ACPP, CD63, KLK 11, ATRN, GP2, PTPRN2, NPTN, RNASE2, CPE, SERPINA1, DSC2, PTGDS, GRN, LRG1, UMOD, CLU, LOX, ORM1, CD97, and AFM.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: Urinary Glycoproteins Associated with Aggressive Prostate Cancer

Glycoproteins play essential roles in cancer development or progression [16-22]. Most of the FDA-approved biomarkers for cancer diagnosis and monitoring are glycoproteins [16]. They are often present on the cell surface or secreted from cells; therefore, they can be found in body fluids (e.g., serum or urine) and serve as noninvasive biomarkers. For the study of PCa, urinary glycoproteins are appealing targets for several reasons. First, the urine collected immediately after the digital rectal examination (DRE) of PCa patients may contain glycoproteins secreted or shed from tumor tissues that may be associated with the aggressiveness of the cancer. Second, the present inventors' previous research has demonstrated that the majority of cancer-associated glycoproteins identified from prostate tissue samples are more readily detected in patients' urine in higher abundances than in their serum [23, 24]. These studies have laid the foundation for the increased use of urine specimens to identify glycoprotein biomarkers for PCa. While high-throughput analysis of clinical specimens is essential for biomarker discovery, the low protein concentration and interfering compounds in urine make it quite challenging for high-throughput proteomic analysis. Recently, the present inventors' lab has reported an automated sample preparation procedure to process urine samples for proteomics and glycoproteomics analysis with high reproducibility and high throughput [25, 26], which paves the way for analyzing large cohorts of clinical urine samples.

Furthermore, the rapid development of mass spectrometry (MS) technology has also advanced biomarker discovery. Data independent acquisition (DIA) MS works as a powerful

tool offering high throughput and reproducibility for quantitative proteomics [27], which is suitable for analyzing large-scale clinical cohorts. Another advantage of DIA MS is that the acquired data set can be reprocessed to obtain previously unidentified features and make parallel comparisons of data acquired at different times. The acquired DIA data can serve as a digital bank of clinical samples, which would benefit long term biomarker screening and aid in the search for novel biomarkers based on the same samples without having to recollect the data. Therefore, DIA MS was used for quantitative analysis in this study because of its high-throughput, cost-effective and flexible nature.

In this study, a high-throughput and integrated workflow for urinary glycoproteomics analysis was employed. The present inventors used an automated approach for urine sample preparation and glycopeptide isolation [25, 26], coupled with DIA MS, to systematically and effectively conduct the quantitative analysis of glycopeptides derived from urine in order to discover unique urinary glycoproteins distinguishing aggressive PCa from non-aggressive PCa. The present inventors also compared and evaluated a combinatorial approach with candidate urinary glycopeptides and serum PSA, which may improve performance for aggressive PCa diagnosis.

### Materials and Methods

Chemicals and reagents. C4 resin beads (35 μm, 300 Å) were purchased from Separation Methods Technologies (Newark, DE). Oasis MAX resins and Sep-Pak C18 resins were obtained from Waters (Milford, MA). Sequencing-grade trypsin and Lys-C were acquired from Promega (Madison, WI). Other chemicals including urea, ammonia bicarbonate (AB), acetonitrile (ACN), trifluoroacetic acid (TFA), triethyl ammonium bicarbonate (TEAB), tris (2-carboxyethyl) phosphine (TCEP), iodoacetamide, and triethylammonium acetate were purchased from Sigma Aldrich (St. Louis, MO). Indexed retention time (iRT) standards (a mixture of eleven peptides) were purchased from Biognosys Inc (Zurich, Switzerland).

Automated tryptic digestion of human urine samples. A discovery cohort containing post-digital rectal examination (DRE) urine samples from 74 AG PCa patients (Gleason score≥8) and 68 NAG PCa patients (Gleason score=6) as well as a validation cohort consisting of 77 post-DRE urine samples (40 AG PCa and 37 NAG PCa) were collected by the Department of Urology at Johns Hopkins University School of Medicine with approval from the Institutional Review Board of Johns Hopkins University under informed consent. Detailed information on the clinical urine specimens is listed in Table S1 (not shown).

The urine samples (500 μL) were desalted and protease digested on Versette (Thermo Scientific, Waltham, MA) according to the automated procedures that the present inventors published previously [25]. In brief, each aspiration/dispense cycle was performed in approximately two minutes at room temperature. C4-tips were fabricated with 30 mg of C4 resin beads packed into each tip and conditioned with 50% ACN containing 0.1% TFA followed by 0.1% TFA (10 cycles each). Next, urine samples (500 μL) were acidified (pH<3) and then loaded onto the C4-tips (90 aspiration/dispense cycles). The tips were rinsed with 0.1% TFA followed by 100 mM triethyl ammonium bicarbonate (TEAB) to remove unbound and contaminant material (10 cycles each). Proteins binding onto the C4-tips were reduced with 10 mM Tris 2-carboxyethyl phosphine (TCEP) in 50mM TEAB buffer (pH 8.2) at room temperature and alkylated with 15 mM iodoacetamide in the dark (20 cycles each). Proteins were digested (1:40 enzyme/protein) by Lys-C for one hour (30 cycles) followed by trypsin digestion for another six hours (120 cycles) in 50 mM TEAB buffer containing 30% ACN to directly recover digested peptides from C4-tips to solution. The C4-tips were subsequently rinsed twice with 50% ACN containing 0.1% TFA to elute the remaining digested peptides into the solution. Peptide mixtures were dried down and stored at −20° C. until analyzed.

Isolation of N-linked glycosite-containing peptide from human urine samples Intact glycopeptides were isolated from the peptide mixture for each urine sample according to the present inventors' recently established automated method [26]. Briefly, 6 mg Oasis MAX resins and 20 mg C18 resins were stacked into tips to generate the mix-mode enrichment tip. The tips were sequentially conditioned by 100% ACN, 100 mM Triethylammonium Acetate (TAAB), 95% ACN containing 1% TFA, and 0.1% TFA (20 cycles each). Peptide mixtures from urine samples were dissolved in 0.1% TFA and put in 96-well plate, then loaded onto MAX/C18 tips with 15 cycles of aspirating/dispensing followed by a rinse with 0.1% TFA (10 cycles). Peptides were desalted via binding onto C18. Desalted intact glycopeptides were eluted from C18 to MAX using 95% ACN/1% TFA. Finally, the bound intact glycopeptides were eluted from MAX by 50% ACN/0.1% TFA and dried down. For the removal of N-glycans, intact glycopeptides were dissolved in 100 mM Tris-HCl at pH 8.0 with 2 μL of PNGase F. The mixture was incubated in 37° C. overnight and subjected to C18-cleanup via StageTip method [28]. After removing N-glycans, N-linked glycosite-containing peptides (one tenth of the total glycopeptides enriched from 500 μL urine) were subjected to DIA MS analysis together with index retention time (iRT) peptides in a Q-Exactive HF-X mass spectrometer.

Basic reversed-phase liquid chromatography (bRPLC) fractionation. To build a PCa urine specific spectral library for direct database searching of the DIA data, glycopeptides from 142 human urine samples (discovery cohort) were pooled and fractionated by bRPLC for a deeper coverage of low abundance peptides. The pooled glycopeptides were load onto reversed-phase Zorbax Extend-C18 analytical column (1.8 μm resin, 4.6×100 mm, Agilent Technology, CA), which was installed on an Agilent 1220 Infinity HPLC system. With buffer A (10 mM ammonium formate, pH 10) and buffer B (10 mM ammonium formate in 90% ACN, pH 10), the HPLC gradient was set as follows: 0-2% B for 10 min followed by 2-8% B for 5 min, 8-35% B for 85 min, 35-95% B for 5 min, and 95-95% B for 15 min. Total of 96 fractions were collected in a time-based mode from 16 to 112 min and were concatenated into 24 fractions. The 24 fractions were further consolidated into eight final fractions. The final pooled fractions were dried down and then dissolved in 0.1% FA together with iRT peptides for data-dependent acquisition (DDA) analysis.

LC-MS/MS analysis of glycopeptides. For the DDA MS analysis, all samples were analyzed by a Q-Exactive HF-X mass spectrometer connected to an EASY-nLC 1200 system (Thermo Fisher Scientific). Glycopeptides were directly injected into a 28 cm long self-packed C18 column (1.9 μm/120 Å ReproSil-Pur C18 resin, Dr. Maisch GmbH, Germany) with an integrated PicoFrit emitter (New Objective). Peptides were separated using 88 min gradient from 5% to 40% buffer B (80% ACN and 0.1% formic acid) at a flow rate of 300 nL/min. MS1 was acquired at a resolution of 60,000 from m/z 400 to 1000 with automatic gain control (AGC) set at 1×10⁶ and a max injection time of 60 ms. MS2 scans were performed by higher-energy collisional dissociation (HCD) on the top 20 abundant precursor ions at a resolution of 15,000 with an isolation width of 1.4 m/z and a normalized collision energy (NCE) of 30. The dynamic exclusion was set as 20 s.

DIA MS analysis was performed on the same MS instrument and LC separation gradient was kept consistent with the DDA analysis. The setting of full MS scan was similar as the MS1 scan of DDA, except the resolution was 120,000 under DIA mode. For the DIA MS2 scan, a set of 50 overlapping windows was constructed covering the precursor mass range of 400-1000 Da with a fixed isolation width of 12 m/z. The resolution and AGC was the same as that of a full MS scan with a maximum injection time of 25 ms and NCE of 30.

Construction of a PCa urine specific spectral library using DDA data. For generation of the PCa urine-specific spectral library, glycopeptides enriched from the 142 urine samples (discovery cohort) were pooled together and measured in two ways. The unfractionated samples were analyzed with DDA in three technical replicates. The eight fractions generated by the bRPLC fractionation method were also measured by DDA MS. In addition, glycopeptides enriched from one urine specimen (sample name: P2) were also subjected to DDA analysis. The 12 DDA raw files were searched against a combined database consisting of an iRT fusion protein and human proteins (Swiss-Prot, downloaded on Feb. 20, 2019) via Pulsar algorithm embedded in Spectronaut Pulsar X (Biognosys, Zurich, Switzerland). The parameters for the database search are as follows: an allowance for tryptic peptides of up to two missed cleavages within the length range of 2 to 52 amino acids. Mass tolerance of MS1 and MS2 were set as dynamic with a correction factor of one. Carbamidomethylation of cysteine (C) was set as a fixed modification whereas oxidation of methionine (M) and acetylation of protein N-terminal were selected as variable modifications. Since N-glycosylated asparagine (N) is converted to aspartic acid (D) upon PNGase F treatment, conversion of N to D was set as a variable modification as well. A false discovery rate (FDR) of<1% was required to generate the final peptide spectral library, in which there were 1289 unique glycopeptides of 594 glycoproteins.

Database search and statistical analysis of glycopeptide DIA data. For quantitative analysis of glycopeptides across the urine samples, DIA raw data files were first searched against the aforementioned spectral library for identification of glycopeptides followed by the quantification via Spectronaut Pulsar X. Mass tolerance of MS and MS/MS was set as dynamic with a correction factor of one. Source-specific iRT calibration was enabled with a local (non-linear) RT regression. Cross run normalization was not selected. All quantified glycopeptides were filtered by a Q value cutoff of 0.01 (which corresponds to an FDR of 1%) and decoy peptide sequences were removed.

The present inventors performed normalization on glycopeptides to the total protein amount in individual urine sample then multiplied by the median of the total amount of proteins across samples. The present inventors used WebGestalt for Gene Ontology (GO) cellular

component annotation [29]. Enriched pathways were analyzed using STRING [30]. At the initial discovery phase, glycopeptides identified and quantified in at least one-third of AG or NAG samples were selected. The p-value for each glycopeptide in the discovery cohort was computed between AG and NAG groups using the Mann-Whitney U test and multiple testing by label permutation was used to estimate the false discovery rate (FDR) of candidate marker selection. For each glycopeptide, its discrimination power as an individual marker or in combination with serum PSA through logistic regression was evaluated using receiver operating characteristic (ROC) analysis in three repeated 5-fold cross validations. The mean ROC curves from repeated 5-fold cross validation were depicted and area under the curve (AUC) was computed for the mean ROC curves. The predictive models with cross validation were built using caret (version 6.0-85) in R. ROC curves were generated using pROC (version 1.13). AUC along with 95% confidence interval (95% CI) as well as sensitivity and specificity at the best cutoff point along with 95% CI were obtained via MLeval in R, for which the best cutoff point on the ROC curve has the maximal summed sensitivity and specificity. The generated predictive models were further investigated using another validation cohort.

Abbreviations. Afamin, AFM; aggressive, AG; Alpha-1-acid glycoprotein 1, ORM1; Alpha-1-antitrypsin, SERPINA1; Area Under Curve, AUC; Attractin, ATRN; Carboxypeptidase E, CPE; CD63 antigen, CD63; CD97 antigen, CD97; Clusterin, CLU; Data independent acquisition, DIA; Desmocollin-2, DSC2; digital rectal examination, DRE Food and Drug Administration, FDA; Kallikrein-11, KLK11; Leucine-rich alpha-2-glycoprotein, LRG1; mass spectrometry, MS; Mix-mode anion exchange, MAX; Neuroplastin, NPTN; non-aggressive, NAG; Non-secretory ribonuclease, RNASE2; Pancreatic secretory granule membrane major glycoprotein GP2, GP2; Progranulin, GRN; Prostaglandin-H2 D-isomerase, PTGDS; prostate cancer antigen-3, PCA3; prostate cancer, PCa; Prostate-specific antigen, PSA; Prostatic acid phosphatase, ACPP; Protein-lysine 6-oxidase, LOX; Receptor-type tyrosine-protein phosphatase N2, PTPRN2; Transmembrane Serine Protease 2, TMPRSS2; Uromodulin, UMOD.

### Results and Discussion

Workflow of an integrated urine glycoproteomic analysis. It is essential to establish an experimental workflow that can analyze a large number of specimens with high throughput, high sensitivity, and high reproducibility for biomarker discovery using urine. A previous study demonstrated the feasibility of detecting a glycoprotein difference between aggressive PCa and

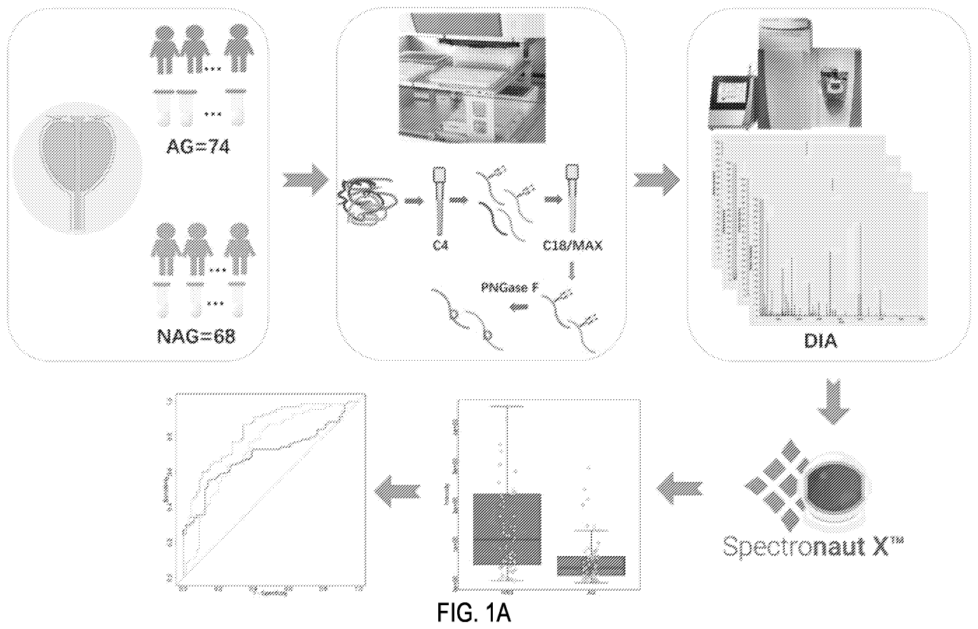

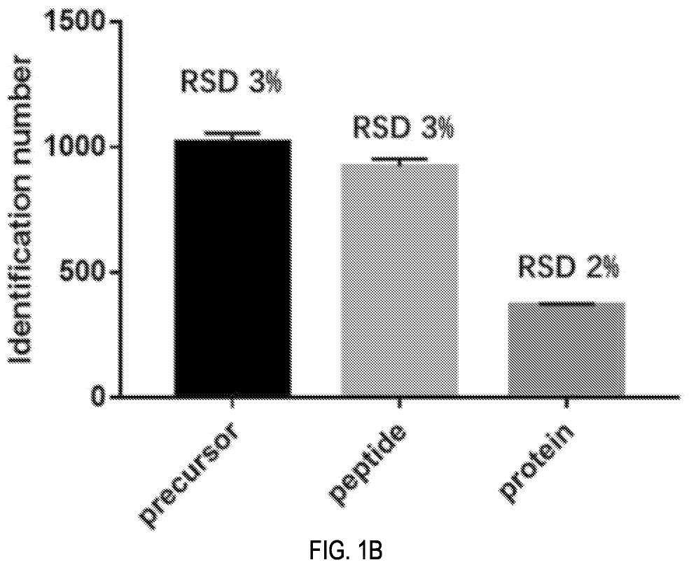

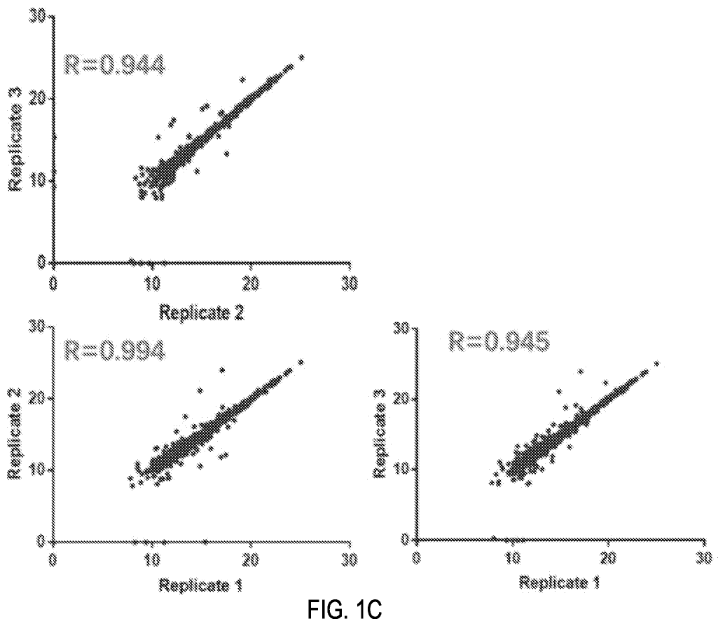

non-aggressive PCa using pooled urine samples from PCa patients [24]. However, the performance of each glycoprotein for differentiating aggressive and non-aggressive PCa was difficult to evaluate using pooled urine samples. Therefore, in this study, the present inventors established an integrated workflow by coupling automated urine sample preparation with DIA MS for the quantitative analysis of N-linked glycosite-containing peptides (referred to as De-N-glycopeptides or glycopeptides for simplicity) derived from the urine samples of 74 AG (Gleason score≥8) and 68 NAG (Gleason score=6) PCa patients (discovery cohort, Table S1 (not shown)). An overview of the experimental workflow is illustrated in FIG. 1A. Table S2 (not shown) shows the major differences between the previous study [24] and the current study in terms of samples, the glycopeptide enrichment method, data acquisition, and quantification approach.

To perform the quantitative proteomic analysis of the enriched glycopeptides using DIA, a PCa urine specific spectral library was generated using the glycopeptides from the discovery cohort via DDA MS (Table S3 (not shown)). The constructed spectral library contained 1,289 unique de-N-glycopeptides corresponding to 594 glycoproteins, thereby allowing for a broad coverage of the PCa-related urine glycoproteome for reliable DIA data analysis. Quantification accuracy and data reproducibility are extremely important for biomarker discovery. Thus, the reproducibility of DIA MS was evaluated by using three replicate injections. The relative standard deviation (RSD) of the identification number of peptide precursors (i.e., the same peptide sequences with different charge states or modifications), peptides, or proteins across the three replicates was 3% or less, which indicates consistency in DIA MS data acquisition (FIG. 1B). To determine reproducibility among replicates, pair-wise correlation was calculated based on the intensity of quantified peptide precursors (FIG. 1C). The correlation between any two replicates was≥0.944 indicating the precision in quantification of the present inventors' DIA MS method.

To investigate the levels of non-enzymatic deamidation generated during sample preparation and assess the false discovery rate of N-linked glycopeptides caused by non-enzymatic deamidation, a control experiment was performed in 20 randomly selected urine specimens from the discovery cohort. The urine proteins were subjected to trypsin digestion followed by enrichment of intact glycopeptides. The enriched intact glycopeptides from each sample were divided into two equal aliquots. One aliquot was directly analyzed by LC-MS/MS without PNGase F treatment. For the other aliquot, peptides were first treated with PNGase F to remove glycans before LC-MS/MS analysis. The identified peptides from the 20 urine specimens are presented in Table S4 (not shown). For the 20 samples without PNGase F treatment, 2132 peptides were identified in total, of which 109 peptides (5.1%) were modified by deamidation and only 5 out of 109 deamidated peptides (4.6%) contained the NXS/T sequence. The result indicates that the identification rate of false N-linked glycopeptides (generated by nonenzymatic deamidation in NXS/T motif) is low (0.2%)). For the PNGase F treated peptides from the same 20 urine samples, 2692 peptides were identified, in which 1652 peptides (61.4%) were modified by deamidation (Table S4 (not shown)). Among the 1652 peptides modified by deamidation, 1458 (88%) of them contained an NXS/T motif in their peptide sequences. Therefore, the present inventors can conclude that most of the deamidated peptides, particularly the deamidated peptides with NXS/T motif, were generated due to removal of glycans using PNGase F.

Overview of the quantified glycopeptides in the discovery cohort. For the DIA MS analysis of each clinical urine sample, glycopeptides enriched from the 142 samples (discovery cohort) were analyzed. In total, 889 glycopeptides originating from 549 glycoproteins were identified and quantified in this study at an FDR of<1% for both proteins and peptides (Table S5 (not shown)). The present inventors further investigated the cellular component of the identified proteins based on the GO annotation [33]. Consistent with previous reports [9, 34], a majority of glycopeptides identified from urine were proteins derived from membrane (373 glycopeptides, 67.9%), extracellular space (312 glycopeptides, 56.8%), or otherwise secreted (304 glycopeptides, 55.4%), indicating that most of the glycopeptides were originated from glycoproteins secreted or shed from tissues. For the biological pathway analysis, the most significantly enriched pathways were neutrophil degranulation, innate immune system, immune system, and extracellular matrix organization.

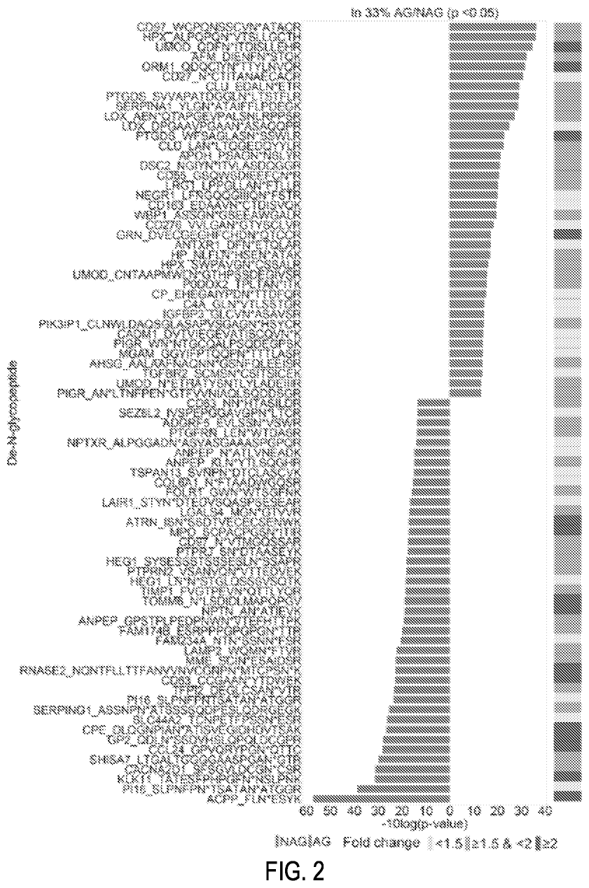

Quantitative analysis of the urinary glycoproteins. To discover urinary glycoproteins associated with aggressive PCa, a two-tier screening approach of candidate selection was used to narrow down the present inventors' initial targets. The first-tier retained glycopeptides quantified in at least one-third of the AG or NAG samples. The second-tier was to filter further and keep only those significantly changed between AG and NAG samples with p<0.05. In total, 79 glycopeptides were identified (Table S6 (not shown)), where 38 increased and 41 decreased in AG group relative to NAG group (FIG. 2), were selected for further evaluation. Among the 79 glycopeptides with significant changes, 54 glycopeptides had at least a 1.5-fold change between AG and NAG groups (Table S7 (not shown) and the right panel of FIG. 2) with an estimated FDR of 0.25 based on label permutation.

Determining the utilities of urinary glycoproteins for the detection of aggressive PCa. To evaluate the discrimination power of the differentially expressed glycoproteins in distinguishing AG PCa from NAG PCa, the ROC curves were generated and AUC were calculated based on predictive models of logistic regression with three repeated 5-fold cross validation for the 54 glycopeptides, where 29 showed decreased levels (Table S7 (not shown)) and 25 showed increased levels (Table S7 (not shown)) in AG PCa relative to NAG PCa. From the ROC results, a total of 20 candidates were selected of which 9 were decreased and 11 were increased in AG PCa samples compared to NAG PCa samples (Tables 2 and 3). In this study, serum PSA concentration obtained from clinical testing served as a reference to determine if candidates were comparable to serum PSA result or could be used in combination with the serum PSA to further improve the discrimination power towards AG PCa. Detailed information on the selected 20 candidates is in Table 4.

Among the 29 glycopeptides showing lower expression in AG PCa, the present inventors found glycopeptide FLN*ESYK (SEQ ID NO:1) from ACPP (Prostatic acid phosphatase, * indicates the glycosylation site) showed the best performance. ACPP is a prostate specific protein with at least fifty-fold higher mRNA expression levels in prostate tissue compared to other tissues [35]. Moreover, decreased expression level of ACPP has been found in the tumor tissues of aggressive PCa patients compared to non-aggressive PCa patients based on quantitative glycoproteomic study [23] and immunohistochemistry analysis of ACPP on the cancer slides of PCa patients [36]. ACPP acts as a tumor suppressor of PCa through dephosphorylation of ERBB2 (receptor tyrosine-protein kinase erbB-2) and deactivation of MAPK-mediated (mitogen-activated protein kinase) signaling [37, 38]. Decreased ACPP expression correlates with the activation of downstream MAPK signaling resulting in PCa progression as well as androgen independent growth of PCa cells [37, 38].

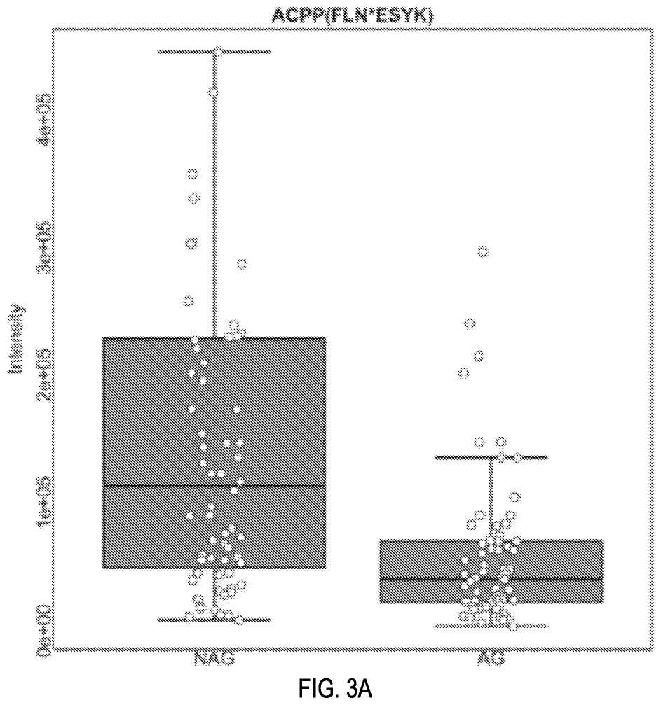

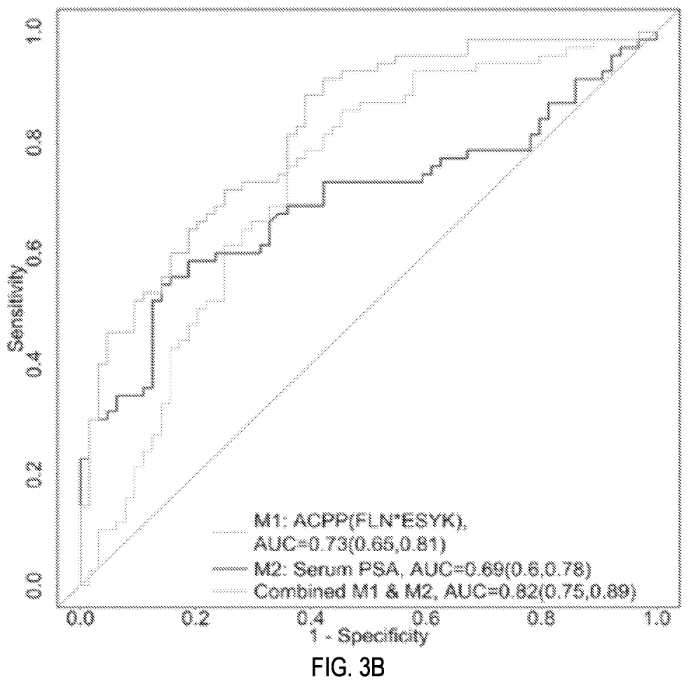

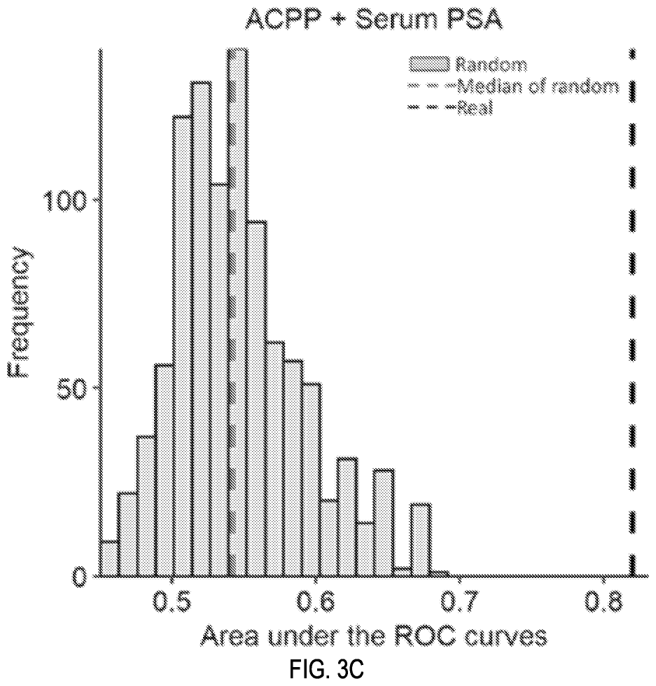

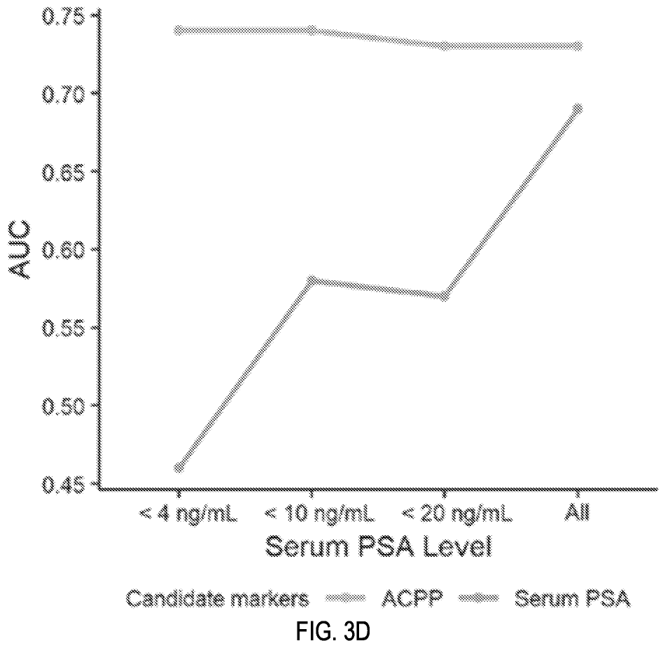

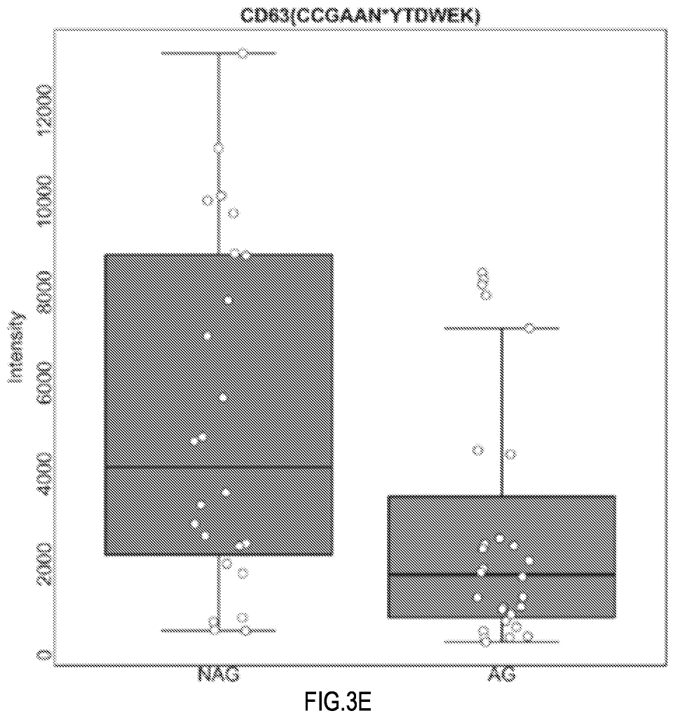

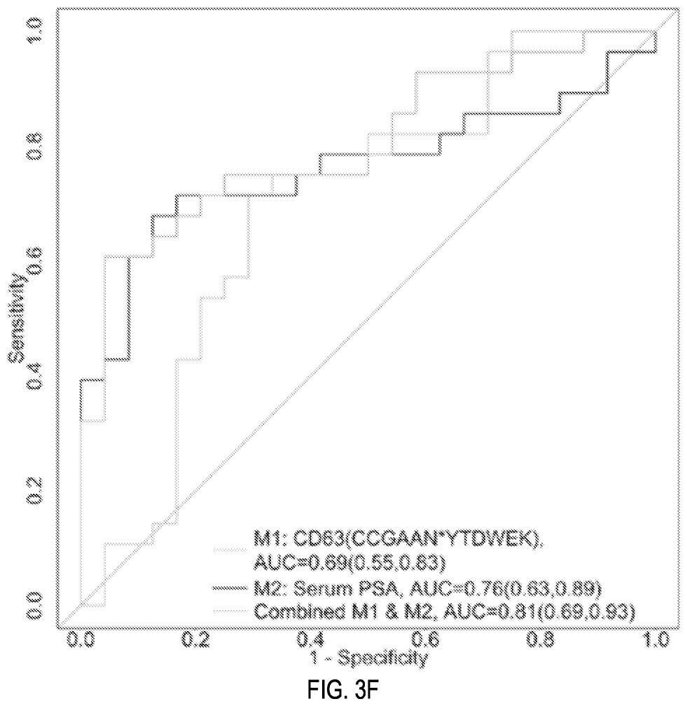

ACPP was initially discovered as a serum biomarker for PCa instead of a urinary biomarker. Serum ACPP was measured by its elevated activity in the patient with PCa rather than by protein abundance [38, 39]. However, the assays to measure the activity of the serum ACPP were unstable in room temperature causing technical variations [40, 41]. Consequently, when serum PSA emerged and demonstrated better accuracy in detecting PCa [42], serum ACPP soon fell to disfavor. In the current study, the present inventors discovered the prognostic value of glycopeptide from urinary ACPP. Glycopeptide FLN*ESYK (SEQ ID NO:1) of ACPP was identified in 70 of AG and 64 of NAG urine samples, with intensity significantly decreased in AG PCa group (fold change=2.56 with p<0.01, Table 4 and FIG. 3A). As shown in FIG. 3B, urinary ACPP had a better predictive power with AUC of 0.73 (95% CI, 0.65 to 0.81) compared to serum PSA with AUC of 0.69 (95% CI, 0.6 to 0.78) (Table 2 and FIG. 3B). Since ACPP (low in AG PCa samples, where median of AG=40547.17 and median of NAG=113063.9) and serum PSA (high in AG PCa samples, where median of AG=7.8 and median of NAG=4.5) had opposite expression profiles with a negative Spearman's correlation of −0.023 suggesting that they may provide complementary information for AG PCa diagnosis. Therefore, a two-signature panel consisting of ACPP and serum PSA was examined. The present inventors found the panel provided better diagnostic accuracy by improving the AUC to 0.82 (95% CI, 0.75 to 0.89) (FIG. 3B and Table 2). To ensure the performance of the panel (urinary ACPP+serum PSA), the present inventors generated 1000 random combined signature sets using label permutation and computed the AUC for each built random model (FIG. 3C). The random models generated a median AUC of 0.55, which was lower and clearly separated from the present inventors' real model (AUC=0.82).

To investigate the effect of serum PSA concentration on the performance of urinary ACPP in detecting AG PCa, ROC analysis was conducted for urinary ACPP and serum PSA at different serum PSA cutoffs. As shown in FIG. 3D, the performance of urinary ACPP is quite consistent across different cutoff points with the AUC ranged from 0.73 to 0.74. On the contrary, the performance of serum PSA varied at different cutoff values with the AUC ranged from 0.45 to 0.69. It has demonstrated limited discrimination power of serum PSA towards AG PCa detection with serum PSA values<20 ng/mL (FIG. 3D). Although further investigation is required, the aforementioned result indicates that the performance of urinary ACPP is independent of serum PSA concentrations. Thus, urinary ACPP may be useful in supplementing serum PSA test for the detection of AG PCa at serum PSA ranges less than 20 ng/mL.

Another down-regulated candidate glycopeptide of interest is CD63 (CD63 antigen). CD63 is one of the widely accepted exosomal markers that belongs to the transmembrane 4 superfamily (TM4SF) [43]. Protein complexes formed by TM4SF members are associated with beta-1 integrin and contribute to cell motility, which plays an important role in tumor progression [43, 44]. In the present inventors' study, glycopeptide (CCGAAN*YTDWEK) (SEQ ID NO:6) from CD63 was identified and its intensity was 2.2 times lower in AG PCa urine specimens comparing to NAG PCa urine specimens (p<0.05, FIG. 3E). FIG. 3F shows that the glycopeptide has the ability to differentiate AG PCa from NAG PCa with an AUC of 0.69 (95% CI, 0.55 to 0.83). When combined with serum PSA, the AUC was further improved (0.81, 95% CI of 0.69 to 0.93, Table 2).

Besides ACPP and CD63, glycopeptides from other proteins such as ATRN (Attractin), GP2 (Pancreatic secretory granule membrane major glycoprotein GP2), KLK11 (Kallikrein-11), PTPRN2 (Receptor-type tyrosine-protein phosphatase N2), NPTN (Neuroplastin), CPE (Carboxypeptidase E), and RNASE2 (Non-secretory ribonuclease), also showed good performance in detecting AG PCa when combined with serum PSA (Table 2). In addition, TMPRSS2 (Transmembrane Serine Protease 2) is another important PCa-specific protein identified in this study. TMPRSS2 is an androgen-responsive gene. Its fusion to ERG contributes to the development of androgen-independence in PCa, resulting in cancer progression

including invasion and metastasis [45]. Genetic detection of this type of fusion in urine specimens has entered clinical practice [46-48]. In this study, the present inventors found the expression levels of LN*TSAGNVDIYK (SEQ ID NO:21) from TMPRSS2 was 1.7-fold decreased in AG PCa urine samples (n=25) relative to NAG PCa urine samples (n=22) with a p-value of 0.13. Usually, the exon 1 or 2 of TMPRSS2 is fused to exon 2 or 4 of ERG during TMPRSS2:ERG fusion [45]. However, the glycosite, N213, on LN*TSAGNVDIYK (SEQ ID NO:21) is located after the fusion position. The present inventors speculate that the TMPRSS2:ERG fusion may lead to the decrease of LN*TSAGNVDIYK (SEQ ID NO:21) expression. Therefore, a decrease in the level of LN*TSAGNVDIYK (SEQ ID NO:21) in AG PCa urine samples may be explained by TMPRSS2:ERG fusion more frequently occurred in AG PCa samples. Further studies are needed to investigate this hypothesis. Nonetheless, the present inventors' finding may provide a new angle for studying the TMPRSS2:ERG fusion.

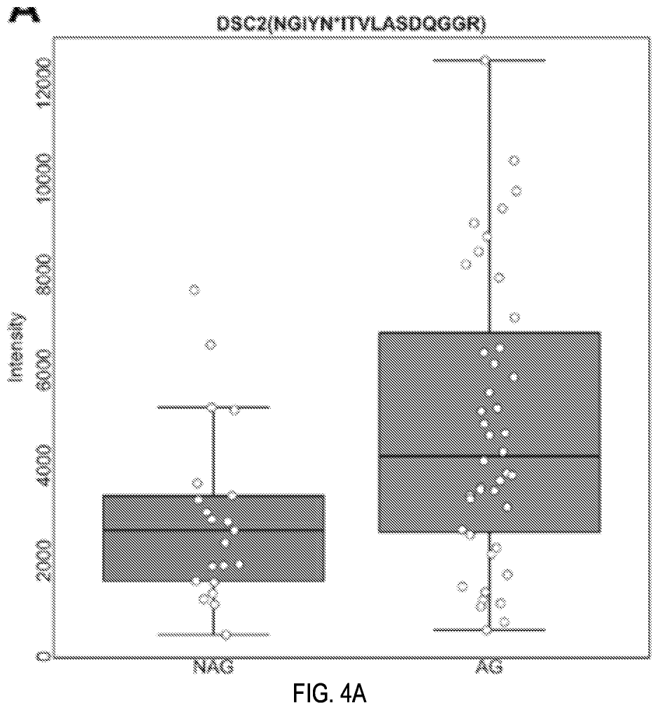

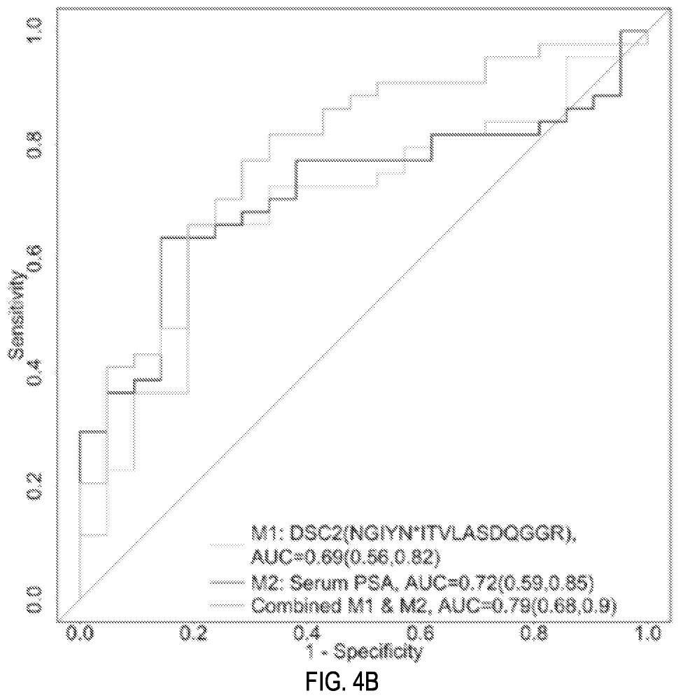

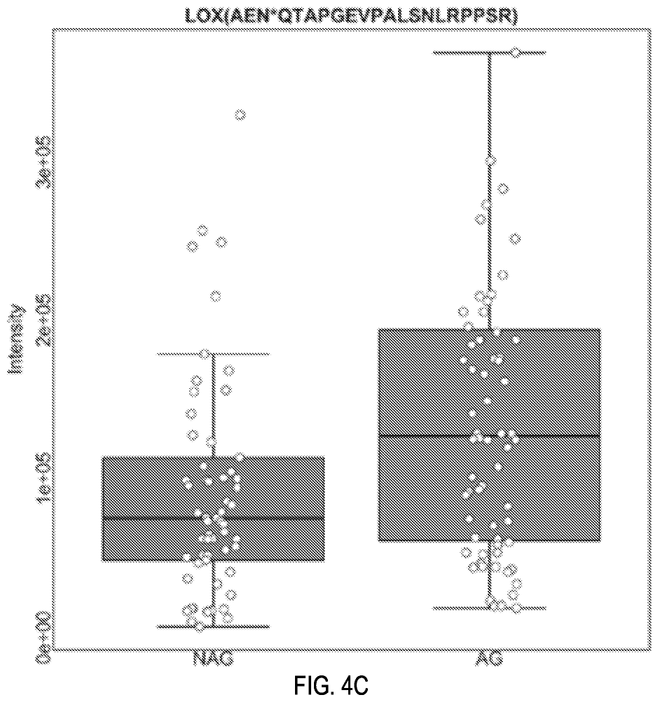

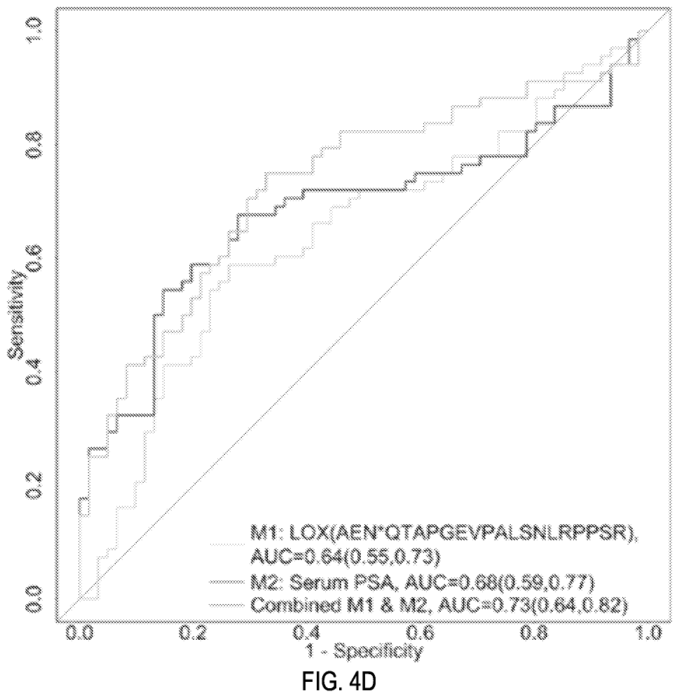

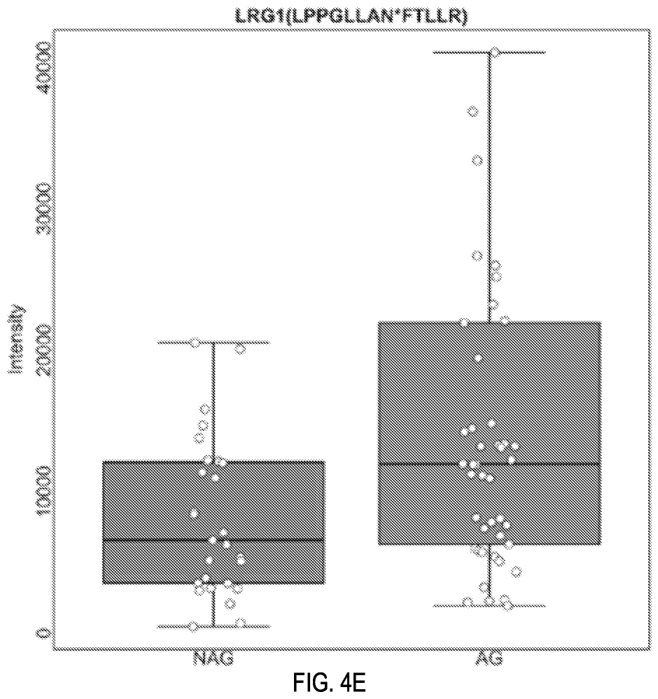

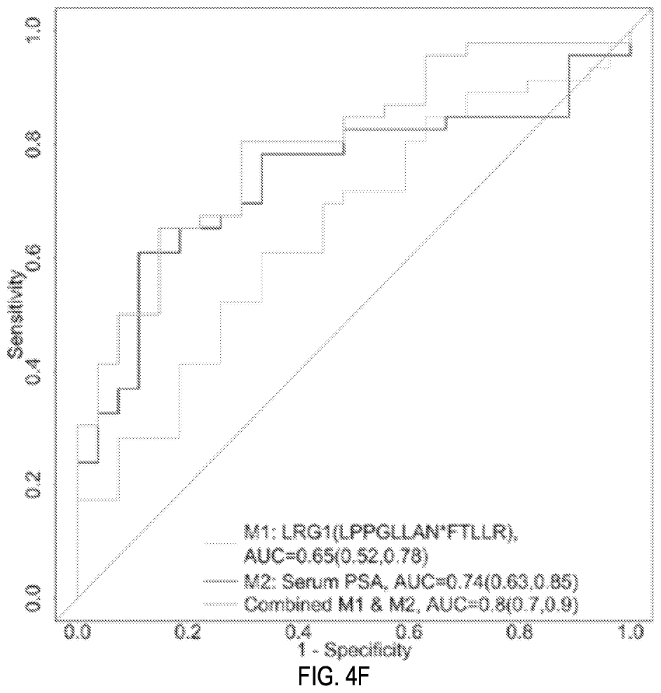

Apart from the aforementioned down-regulated glycopeptides, the present inventors also explored the up-regulated candidate glycopeptides including NGIYN*ITVLASDQGGR (SEQ ID NO:14) from DSC2 (Desmocollin-2), AEN*QTAPGEVPALSNLRPPSR (SEQ ID NO:2) from LOX (Protein-lysine 6-oxidase), and LPPGLLAN*FTLLR (SEQ ID NO:15) from LRG1 (Leucine-rich alpha-2-glycoprotein), as shown in FIG. 4. DSC2 belongs to the demecolcine protein subfamily and is the major component of desmosomes. Desmosomes are involved in establishing and maintaining cell-cell adhesion and are critical for the development, differentiation, and maintenance of normal human tissues [49]. The loss of cell-cell adhesion is frequently associated with the progression of PCa to a metastatic state. Previous research has found aberrant expression of DCS2 in several types of cancers by possible involvement in tumor progression [50]. In the present inventors' study, the present inventors observed an elevated expression profile of the glycopeptide, NGIYN*ITVLASDQGGR of DSC2 (SEQ ID NO:14), in AG PCa urine samples (FIG. 4A). NGIYN*ITVLASDQGGR (SEQ ID NO:14) of DSC2 generated an AUC of 0.69 (95% CI, 0.56 to 0.82) as an individual signature. However, an improvement was noticed when combined with serum PSA with an AUC of 0.79 (95% CI, 0.68 to 0.9) (FIG. 4B and Table 3). LOX is another glycopeptide of interest since it is reported to be associated with PCa [51]. The present inventors observed the level of AEN*QTAPGEVPALSNLRPPSR (SEQ ID NO:2) from LOX was higher in AG PCa compared to NAG PCa (FIG. 4C). The ROC analysis included AEN*QTAPGEVPALSNLRPPSR (SEQ ID NO:2) of LOX and serum PSA indicating that the combination of the two enhanced the separation of AG PCa from NAG PCa (AUC of 0.73, 95% CI of 0.64 to 0.82) in comparison with using LOX (AUC of 0.64, 95% CI of 0.55 to 0.73) and serum PSA (AUC of 0.68, 95% CI of 0.59 to 0.77) individually (FIG. 4D and Table 3). Furthermore, LRG1 (Leucine-rich-alpha-2-glycoprotein-1), an inflammatory protein in human serum participates in the immune response [52], was identified as an up-regulated glycopeptide in this study. LRG1 was previously recognized as a new oncogene-associated protein promoting dysfunctional vessel growth [53]. The up-regulation of LRG1 has been reported to be associated with progression and angiogenesis of multiple cancers [54-56]. Here, LPPGLLAN*FTLLR (SEQ ID NO:15) from LRG1 was expressed twice more in AG PCa than NAG PCa urine samples (FIG. 4E). A combined use of LRG1 and serum PSA generated an AUC of 0.8 (95% CI, 0.7 to 0.9) (FIG. 4F and Table 3) suggesting the potential of LRG1 as a urinary glycoprotein for aggressive PCa. In addition to DSC2, LOX and LRG1, glycopeptides from other glycoproteins including CLU (Clusterin), SERPINA1 (Alpha-1-antitrypsin), ORM1 (Alpha-1-acid glycoprotein 1), PTGDS (Prostaglandin-H2 D-isomerase), GRN (Progranulin), UMOD (Uromodulin), AFM (Afamin) and CD97 (CD97 antigen), were also found to be significantly (p<0.05) elevated in AG PCa urine specimens and the capacity in group separation was evaluated by ROC analysis (Table 3).

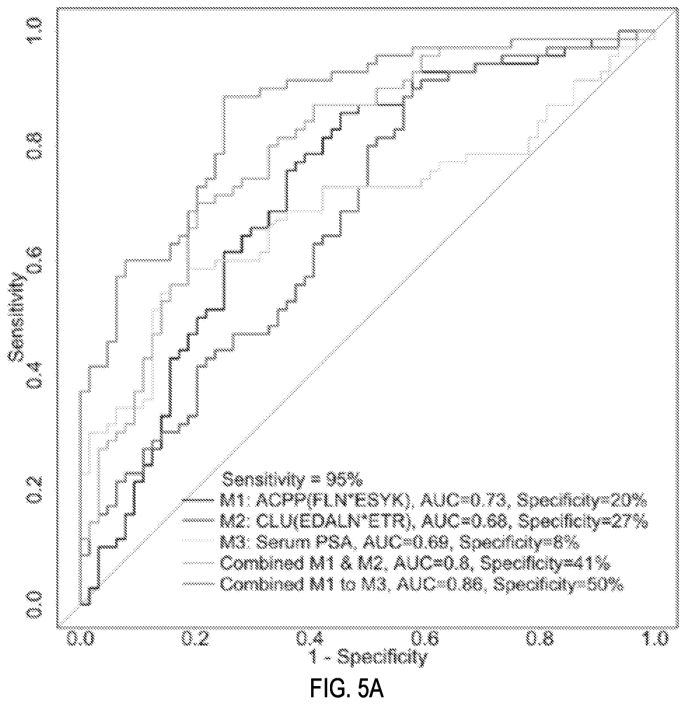

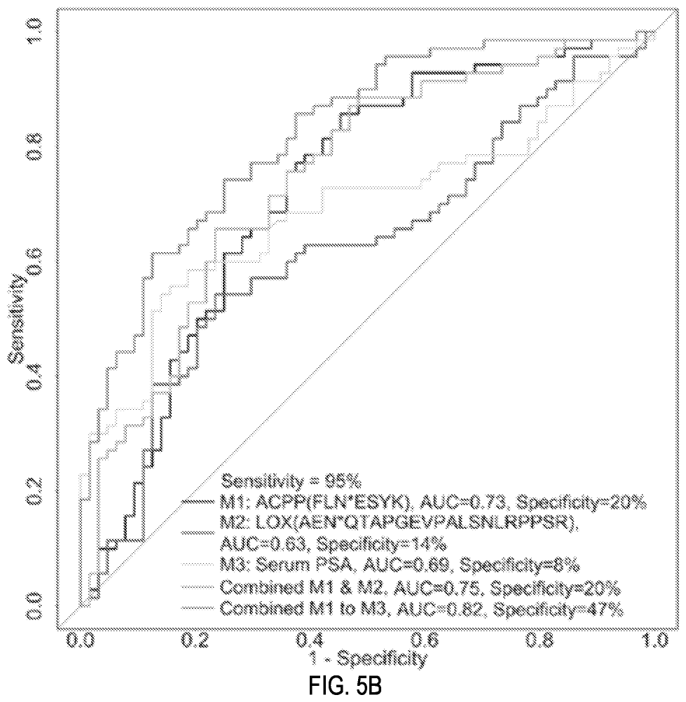

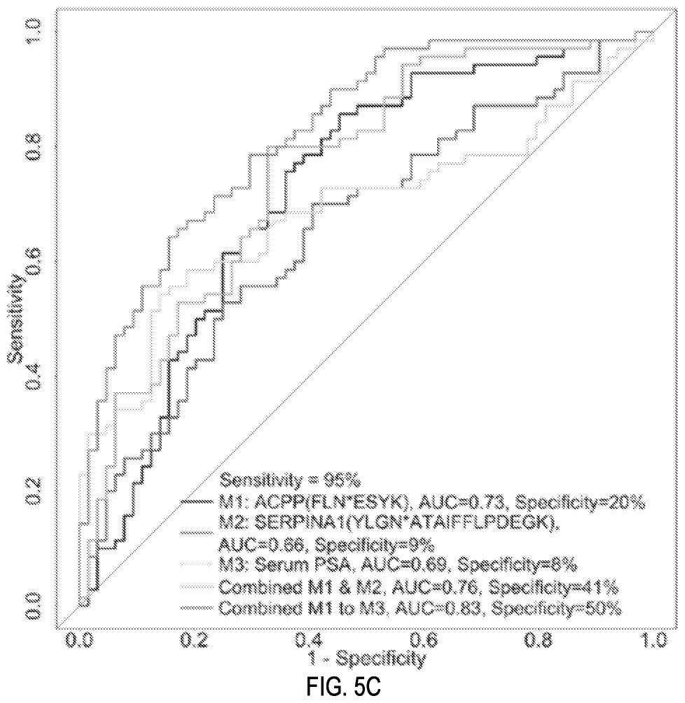

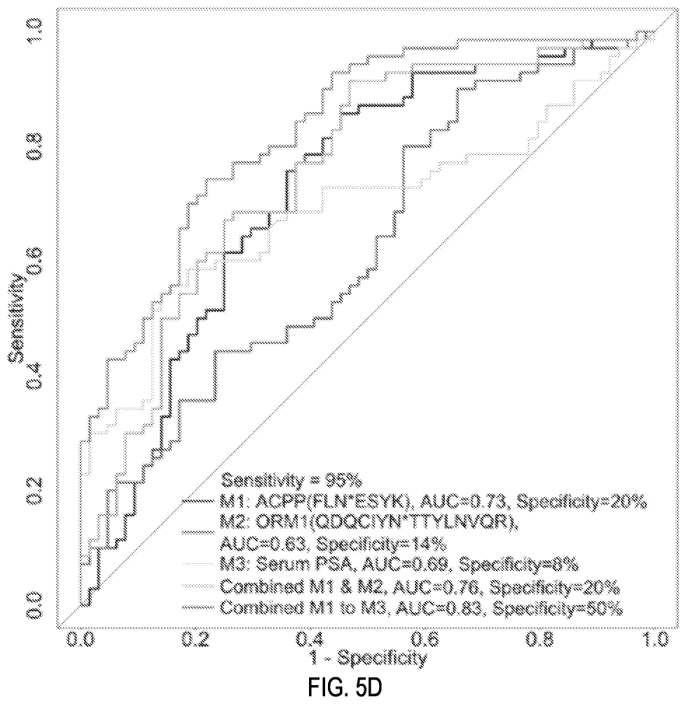

The combined performance of two urinary glycoproteins and serum PSA for the detection of AG PCa. After evaluating the glycopeptide signatures individually and in combination with serum PSA, the present inventors further investigated the potential of combining a down-regulated glycopeptide and an up-regulated glycopeptide since this may improve the clinical utility of these candidate glycopeptides. The present inventors selected urinary ACPP as the primary down-regulated candidate glycopeptide because it had the best performance among the candidate glycopeptides. To directly compare the performance of urinary ACPP with different up-regulated candidate glycopeptides, the present inventors fixed the sensitivity at 95% and then compared the specificity. By setting a very high sensitivity, even at the cost of reduced specificity, would also fulfill the need in clinical practice for lowering misdiagnosis of patients with aggressive PCa. The ROC results of different panels are shown in Table 5, where the four panels demonstrated relatively better performance in distinguishing AG PCa from NAG PCa (higher AUC and higher specificity at 95% sensitivity) are presented in FIG. 5.

Among all the two-signature panels (i.e., the combination of urinary ACPP and an up-regulated glycopeptide), urinary ACPP combined with urinary CLU had the best performance (AUC=0.8) achieving a specificity of 41% at 95% sensitivity (FIG. 5A and Table 5). By adding serum PSA into the panel, the AUC was improved to 0.86 and specificity was increased to 50% at 95% sensitivity (FIG. 5A and Table 5). While serum PSA itself generated an AUC of 0.69, and the specificity was only 8% at 95% sensitivity. Since ACPP and CLU were detected in more than 91% of the samples, further clinical use of the combined signature panel is possible. The combination of urinary ACPP, urinary LOX, and serum PSA as a three-signature panel also displayed a good capacity in differentiating AG from NAG PCa (AUC=0.82, FIG. 5B and Table 5). Furthermore, urinary ACPP combined with urinary SERPINA1 (FIG. 5C and Table 5) and urinary ACPP combined with urinary ORM1 (FIG. 5D and Table 5) both achieved an AUC of 0.76. Additional improvement in discrimination power was observed when serum PSA was included; the AUC increased to 0.83 with specificity reached to 50% at 95% sensitivity (FIGS. 5C-D). These results demonstrate that a combined signature panel composed of one up, one down-regulated glycopeptides from urinary glycoproteins, and serum PSA has the ability to distinguish AG and NAG PCs patients, where the two glycopeptide signatures serve as adjuncts to serum PSA test to gain improved discrimination power. In conclusion, three-signature panels discovered using quantitative glycoproteomic strategy showed better performance than individual signatures for the detection of AG PCa.

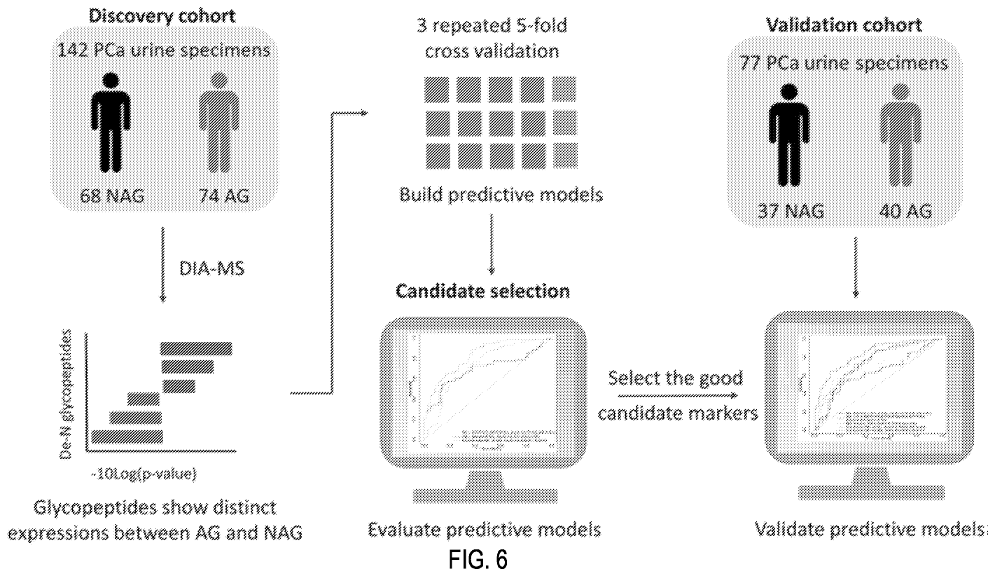

Validation of the glycopeptide signatures using a validation cohort. A validation cohort composed of 40 AG and 37 NAG PCa patients was analyzed to further assess the performance of the identified glycopeptides and the predictive models from the discovery cohort (FIG. 6). Among the 20 candidate glycopeptides selected based on ROC analysis in discovery set (Table 4), 13 of them showed the same trend in their expression profiles as they did in discovery cohort (Table 7).

Next, the present inventors evaluated the performance of the candidate glycopeptides that can distinguish AG from NAG PCa during the discovery phase, including glycopeptides from ACPP, CD63, DSC2, LOX and LRG1 (FIGS. 3 and 4), using the validation cohort. Among them, the predictive power of glycopeptides from DSC2 and LRG1 decreased in the validation cohort (Table 9), suggesting that further rigorous validation is needed to evaluate their potential diagnosis utility for aggressive PCa. Nevertheless, ACPP, CD63 and LOX performed consistently between the discovery and validation cohorts, either as individual biomarkers or in combination with serum PSA (Table 9). For example, ACPP remained as a reliable candidate biomarker for distinguishing AG from NAG PCa, especially when combined with serum PSA to achieve an AUC of 0.83 (95% CI of 0.74 to 0.92) in the validation cohort (Table 1 and Table 9), which was comparable to that of the discovery cohort (AUC=0.82, 95% CI of 0.75 to 0.89).

TABLE 1
Performance of different panel of candidate biomarkers in discovery cohort (74 AG
and 68 NAG), validation cohort (set 1: 40 AG and 37 NAG; set 2: 40 AG and 13 NAG).

| | Area under the ROC curves | | | | |
|---|---|---|---|---|---|
| | (95% confidence interval) | | | | |
| | | | Validation cohort | | |
| Panel of | | | 40 AG and | 40 AG and | |
| candidate | | | 37 NAG | 13 NAG | |
| biomarkers | discovery cohort | | (set 1) | (set 2) | |
| ACPP & Serum PSA | 0.82 | (0.75, 0.89) | 0.83 (0.74, 0.92) | 0.8 | (0.67, 0.93) |
| ACPP & CLU & Serum PSA | 0.86 | (0.8, 0.92) | 0.85 (0.76, 0.94) | 0.76 | (0.6, 0.92) |
| ACPP & LOX & Serum PSA | 0.82 | (0.75, 0.89) | 0.85 (0.76, 0.93) | 0.81 | (0.69, 0.93) |
| ACPP & SERPINA1 & Serum PSA | 0.83 | (0.76, 0.9) | 0.84 (0.75, 0.93) | 0.82 | (0.7, 0.94) |
| ACPP & ORM1 & Serum PSA | 0.83 | (0.76, 0.9) | 0.82 (0.72, 0.91) | 0.82 | (0.71, 0.94) |

The present inventors further evaluated the performances of the predictive models composed of three candidate biomarkers. Combining ACPP and serum PSA with one of the elevated urinary signatures (CLU, LOX, ORM1, or SERPINA1) showed good performances relative to the other panels for detecting AG PCa in the discovery set (Table 5 and FIG. 5). The four panels were successfully validated and consistent performances were observed between the discovery and validation cohorts (Table 10 and Table 1). Taking the three-signature panel consisting of urinary ACPP, urinary LOX, and serum PSA as an example, the AUCs were 0.82 and 0.85 in the discovery cohort and the validation cohort, respectively, indicating the stable performance of the predictive model of this panel.

However, the present inventors later found that 24 NAG urine samples in the validation sample cohort were collected from a subset of patients in the discovery cohort. Since the entire sample preparation and DIA-MS analysis processes were carried out independently for the discovery and validation cohorts, the present inventors still present the evaluation results using the entire validation cohort (referred to as validation set 1). By removing the overlapped patient samples, a subset of the validation set consisting of 40 AG and 13 NAG urine samples was used (referred to as validation set 2) to conduct another evaluation (Table 1 and Table 8-13). As shown in Table 1, the discrimination power of ACPP combined with serum PSA was slightly lower using validation set 2 (AUC=0.8), which was possibly related to the smaller sample size of the second validation set. An AUC of 0.81 was found for the three-signature panel consisting of urinary ACPP, urinary LOX, and serum PSA in validation set 2 (Table 1 and Table 10). A similar outcome was observed for the five individual candidate biomarkers comparing validation set 1 to validation set 2 (Table 9). Collectively, novel panels of candidate biomarkers for aggressive PCa were discovered, and they performed consistently in an independent validation cohort. For clinical applications of the biomarker panels, the next phase of the present inventors' study would be to validate the panels using larger urine sample cohorts from multi-centers to further assess their reliability.

### Conclusion

Despite the prevalence of PCa, there is still a lack of biomarkers for identifying aggressive PCa. Therefore, developing a noninvasive test for the early detection of aggressive PCa is necessary. The aim of this study is to detect glycopeptides of urinary glycoproteins associated with aggressive PCa. By applying a high throughput and integrated workflow involving automation in the urine sample preparation, DIA MS, and quantitative analysis of the urine glycoproteome, the present inventors were able to evaluate the performance of glycopeptides from the 142 urine samples (discovery cohort) for the aim of detecting aggressive PCa.

Based on the present inventors' analysis, 79 glycopeptides were significantly altered between AG and NAG samples (p<0.05), 54 of which having at least a 1.5-fold change. Moreover, 20 glycopeptides were identified as candidates associated with aggressiveness in PCa. Glycopeptide FLN*ESYK (SEQ ID NO:1) from ACPP showed the best performance as an individual candidate signature compared to other candidates; further improvement was observed when combined with the traditional serum PSA. In addition, the performance of urinary ACPP is independent of serum PSA concentrations; thus, it can serve as an adjunct to serum PSA for the detection of aggressive PCa particularly for patients with lower level of serum PSA. Glycopeptides from CD63 and LOX also showed potential as noninvasive urinary glycoproteomic biomarkers for aggressive PCa with consistent performances across the discovery and validation cohorts. Notably, three-signature panels comprising of urinary ACPP; urinary CLU, LOX, ORM1, or SERPINA1; and serum PSA outperformed individual signatures when it came to detecting AG PCa. The three-signature panel composed of ACPP, CLU and serum PSA can discriminate aggressive PCa at an AUC of 0.86. The predictive models with good performance were further investigated using a validation cohort. Consistent results were found between the discovery and validation cohorts, indicating the reliability of the candidates.

The present inventors' study highlights the application of a high-throughput and highly reproducible automated urine glycopeptide preparation platform coupled with DIA MS for the discovery of glycoproteins associated with aggressive PCa. While the novel panels of multiple signatures discovered in this study demonstrate the potential of characterizing and detecting the aggressiveness of PCa, substantial work is still needed before they can be used for clinical applications. The next phase of the present inventors' research will include the following: (1) Validate the urinary candidate biomarkers using a large-scale cohort. (2) Perform multi-center validation. (3) Make systematic comparisons of the glycoproteomic candidate biomarkers found in this study with other urinary biomarkers for PCa (e.g., urinary RNA biomarker PCA3) to investigate whether they can supplement each other to generate a new panel of noninvasive urinary biomarkers with improved discrimination power. Furthermore, aberrant glycosylation has been recognized as a hallmark in oncogenic transformation and plays an important role in cancer development and progression. Thus, studying glycosylation patterns will help in biomarker discovery. For instance, fucosylated PSA displays a better predictive power to differentiate aggressive from non-aggressive PCa [57, 58]. Therefore, the present inventors will also dedicate the present inventors' efforts to investigate the glycosylation forms of the present inventors' glycoproteins in order to further improve the diagnostic accuracy of aggressive PCa.

### References

1. Siegel R L, Miller K D, Jemal A. Cancer statistics, 2019. CA Cancer J Clin. 2019; 69:7-34.
2. Taylor K L, Luta G, Miller A B, Church T R, Kelly S P, Muenz L R, et al. Long-term disease-specific functioning among prostate cancer survivors and noncancer controls in the prostate, lung, colorectal, and ovarian cancer screening trial. J Clin Oncol. 2012; 30:2768-75.
3. Cortese R, Kwan A, Lalonde E, Bryzgunova O, Bondar A, Wu Y, et al. Epigenetic markers of prostate cancer in plasma circulating DNA. Hum Mol Genet. 2012; 21:3619-31.
4. Tomlins S A, Aubin S M, Siddiqui J, Lonigro R J, Sefton-Miller L, Miick S, et al. Urine TMPRSS2:ERG fusion transcript stratifies prostate cancer risk in men with elevated serum PSA. Sci Transl Med. 2011; 3:94ra72.
5. Robert G, Jannink S, Smit F, Aalders T, Hessels D, Cremers R, et al. Rational basis for the combination of PCA3 and TMPRSS2:ERG gene fusion for prostate cancer diagnosis. Prostate. 2013; 73:113-20.
6. Lalonde E, Ishkanian A S, Sykes J, Fraser M, Ross-Adams H, Erho N, et al. Tumour genomic and microenvironmental heterogeneity for integrated prediction of 5-year biochemical recurrence of prostate cancer: a retrospective cohort study. Lancet Oncol. 2014; 15:1521-32.
7. Leyten G H, Hessels D, Jannink S A, Smit F P, de Jong H, Cornel E B, et al. Prospective multicentre evaluation of PCA3 and TMPRSS2-ERG gene fusions as diagnostic and prognostic urinary biomarkers for prostate cancer. Eur Urol. 2014; 65:534-42.
8. Leyten G H, Hessels D, Smit F P, Jannink S A, de Jong H, Melchers W J, et al. Identification of a Candidate Gene Panel for the Early Diagnosis of Prostate Cancer. Clin Cancer Res. 2015; 21:3061-70.
9. Kim Y, Jeon J, Mejia S, Yao C Q, Ignatchenko V, Nyalwidhe J O, et al. Targeted proteomics identifies liquid-biopsy signatures for extracapsular prostate cancer. Nat Commun. 2016; 7:11906.
10. McKiernan J, Donovan M J, O'Neill V, Bentink S, Noerholm M, Belzer S, et al. A Novel Urine Exosome Gene Expression Assay to Predict High-grade Prostate Cancer at Initial Biopsy. JAMA Oncol. 2016; 2:882-9.
11. Jeon J, Olkhov-Mitsel E, Xie H, Yao C Q, Zhao F, Jahangiri S, et al. Temporal Stability and Prognostic Biomarker Potential of the Prostate Cancer Urine miRNA Transcriptome. J Natl Cancer Inst. 2020; 112:247-55.
12. Koo K M, Wee E J, Trau M. Colorimetric TMPRSS2-ERG Gene Fusion Detection in Prostate Cancer Urinary Samples via Recombinase Polymerase Amplification. Theranostics. 2016; 6:1415-24.
13. Puhka M, Takatalo M, Nordberg M E, Valkonen S, Nandania J, Aatonen M, et al. Metabolomic Profiling of Extracellular Vesicles and Alternative Normalization Methods Reveal Enriched Metabolites and Strategies to Study Prostate Cancer-Related Changes. Theranostics. 2017; 7:3824-41.
14. Rittenhouse H, Blase A, Shamel B, Schalken J, Groskopf J. The long and winding road to FDA approval of a novel prostate cancer test: our story. Clin Chem. 2013; 59:32-4.
15. Auprich M, Chun F K, Ward J F, Pummer K, Babaian R, Augustin H, et al. Critical assessment of preoperative urinary prostate cancer antigen 3 on the accuracy of prostate cancer staging. Eur Urol. 2011; 59:96-105.
16. Pinho S S, Reis C A. Glycosylation in cancer: mechanisms and clinical implications. Nat Rev Cancer. 2015; 15:540-55.
17. Gottesman M M, Ling V. The molecular basis of multidrug resistance in cancer: The early years of P-glycoprotein research. Febs Letters. 2006; 580:998-1009.
18. Bellahcene A, Castronovo V, Ogbureke K U E, Fisher L W, Fedarko N S. Small integrin-binding ligand N-linked glycoproteins (SIBLINGs): multifunctional proteins in cancer. Nature Reviews Cancer. 2008; 8:212-26.
19. Ahn J M, Sung H J, Yoon Y H, Kim B G, Yang W S, Lee C, et al. Integrated Glycoproteomics Demonstrates Fucosylated Serum Paraoxonase 1 Alterations in Small Cell Lung Cancer. Molecular & Cellular Proteomics. 2014; 13:30-48.
20. Clark D J, Mei Y P, Sun S S, Zhang H, Yang A J, Mao L. Glycoproteomic Approach Identifies KRAS as a Positive Regulator of CREG1 in Non-small Cell Lung Cancer Cells. Theranostics. 2016; 6:65-77.
21. Fernandes E, Sores J, Cotton S, Peixoto A, Ferreira D, Freitas R, et al. Esophageal, gastric and colorectal cancers: Looking beyond classical serological biomarkers towards glycoproteomics-assisted precision oncology. Theranostics. 2020; 10:4903-28.
22. Hoti N, Lih T S, Pan J B, Zhou Y Y, Yang G L, Deng A, et al. A Comprehensive Analysis of FUT8 Overexpressing Prostate Cancer Cells Reveals the Role of EGFR in Castration Resistance. Cancers (Basel). 2020; 12.
23. Liu Y S, Chen J, Sethi A, Li Q K, Chen L J, Collins B, et al. Glycoproteomic analysis of prostate cancer tissues by SWATH mass spectrometry discovers N-acylethanolamine acid amidase and protein tyrosine kinase 7 as signatures for tumor aggressiveness. Mol Cell Proteomics. 2014; 13:1753-68.
24. Jia W, Chen J, Sun S S, Yang W M, Yang S, Shah P, et al. Detection of aggressive prostate cancer associated glycoproteins in urine using glycoproteomics and mass spectrometry. Proteomics. 2016; 16:2989-96.
25. Clark D J, Hu Y W, Schnaubelt M, Fu Y, Ponce S, Chen S Y, et al. Simple Tip-Based Sample Processing Method for Urinary Proteomic Analysis. Anal Chem. 2019; 91:5517-22.
26. Chen S Y, Dong M M, Yang G L, Zhou Y Y, Clark D J, Lih T M, et al. Glycans, Glycosite, and Intact Glycopeptide Analysis of N-Linked Glycoproteins Using Liquid Handling Systems. Anal Chem. 2020; 92:1680-6.
27. Ludwig C, Gillet L, Rosenberger G, Amon S, Collins B C, Aebersold R. Data-independent acquisition-based SWATH-MS for quantitative proteomics: a tutorial. Mol Syst Biol. 2018; 14:e8126.
28. Rappsilber J, Mann M, Ishihama Y. Protocol for micro-purification, enrichment, pre-fractionation and storage of peptides for proteomics using StageTips. Nat Protoc. 2007; 2:1896-906.
29. Liao Y X, Wang J, Jaehnig E J, Shi Z, Zhang B. WebGestalt 2019: gene set analysis toolkit with revamped UIs and APIs. Nucleic Acids Res. 2019; 47:W199-W205.
30. Szklarczyk D, Gable A L, Lyon D, Junge A, Wyder S, Huerta-Cepas J, et al. STRING v11: protein-protein association networks with increased coverage, supporting functional discovery in genome-wide experimental datasets. Nucleic Acids Res. 2019; 47:D607-D13.
31. Kuhn M. Building Predictive Models in R Using the caret Package. J Stat Softw. 2008; 28:1-26.
32. John C R. MLeval: Machine Learning Model Evaluation. R package version 03. 2020.
33. Ashburner M, Ball C A, Blake J A, Botstein D, Butler H, Cherry J M, et al. Gene ontology: tool for the unification of biology. The Gene Ontology Consortium. Nat Genet. 2000; 25-9.
34. Kuhlmann L, Cummins E, Samudio I, Kislinger T. Cell-surface proteomics for the identification of novel therapeutic targets in cancer. Expert Rev Proteomics. 2018; 15:259-75.
35. Uhlen M, Fagerberg L, Hallstrom B M, Lindskog C, Oksvold P, Mardinoglu A, et al. Tissue-based map of the human proteome. Science. 2015; 347:1260419-.
36. Allsbrook W C, Simms W W. Histochemistry of the prostate. Hum Pathol. 1992; 23:297-305.
37. Chuang T D, Chen S J, Lin F F, Veeramani S, Kumar S, Batra S K, et al. Human prostatic acid phosphatase, an authentic tyrosine phosphatase, dephosphorylates ErbB-2 and regulates prostate cancer cell growth. J Biol Chem. 2010; 285:23598-606.
38. Veeramani S, Yuan T C, Chen S J, Lin F F, Petersen J E, Shaheduzzaman S, et al. Cellular prostatic acid phosphatase: a protein tyrosine phosphatase involved in androgen-independent proliferation of prostate cancer. Endocr Relat Cancer. 2005; 12:805-22.
39. Gutman A B, Gutman E B. An “acid” phosphatase occurring in the serum of patients with metastasizing carcinoma of the prostate gland. J Clin Invest. 1938; 17:473-8.
40. Roy A V, Brower M E, Hayden J E. Sodium thymolphthalein monophosphate: A new acid phosphatase substrate with greater specificity for the prostatic enzyme in serum. Clin Chem. 1971; 17:1093-102.
41. Lowe F C, Trauzzi S J. Prostatic Acid-Phospatase in 1993—Its Limited Clinical Utility. Urol Clin North Am. 1993; 20:589-95.
42. Stamey T A, Yang N, Hay A R, McNeal J E, Freiha F S, Redwine E. Prostate-Specific Antigen as a Serum Marker for Adenocarcinoma of the Prostate. N Engl J Med. 1987; 317:909-16.
43. Hemler M E, Mannion B A, Berditchevski F. Association of TM4SF proteins with integrins: relevance to cancer. Biochim Biophys Acta. 1996; 1287:67-71.
44. Soekmadji C, Russell P J, Nelson C C. Exosomes in prostate cancer: putting together the pieces of a puzzle. Cancers 2013; 5:1522-44.
45. Tomlins S A, Rhodes D R, Perner S, Dhanasekaran S M, Mehra R, Sun W, et al. Recurrent fusion of TMPRSS2 and ETS transcription factor genes in prostate cancer. Science. 2005; 310:644-8.
46. Tomlins S A, Day J R, Lonigro R J, Hovelson D H, Siddiqui J, Kunju L P, et al. Urine TMPRSS2:ERG Plus PCA3 for Individualized Prostate Cancer Risk Assessment. Eur Urol. 2016; 70:45-53.
47. Sanda M G, Feng Z D, Howard D H, Tomlins S A, Sokoll L J, Chan D W, et al. Association Between Combined TMPRSS2: ERG and PCA3 RNA Urinary Testing and Detection of Aggressive Prostate Cancer. Jama Oncology. 2017; 3:1085-93.
48. Lin D W, Newcomb L F, Brown E C, Brooks J D, Carroll P R, Feng Z D, et al. Urinary TMPRSS2:ERG and PCA3 in an Active Surveillance Cohort: Results from a Baseline Analysis in the Canary Prostate Active Surveillance Study. Clin Cancer Res. 2013; 19:2442-50.
49. Dusek R L, Godsel L M, Green K J. Discriminating roles of desmosomal cadherins: beyond desmosomal adhesion. J Dermatol Sci. 2007; 45:7-21.
50. Sun C, Wang L, Yang X X, Jiang Y H, Guo X L. The aberrant expression or disruption of desmocollin2 in human diseases. Int J Biol Macromol. 2019; 131:378-86.
51. Bais M V, Ozdener G B, Sonenshein G E, Trackman P C. Effects of tumor-suppressor lysyl oxidase propeptide on prostate cancer xenograft growth and its direct interactions with DNA repair pathways. Oncogene. 2015; 34:1928-37.
52. Haupt H, Baudner S. Isolation and characterization of an unknown, leucine-rich 3.1-S-alpha2-glycoprotein from human serum. Hoppe Seylers Z Physiol Chem. 1977; 358:639-46.
53. Wang X M, Abraham S, McKenzie J A G, Jeffs N, Swire M, Tripathi V B, et al. LRG1 promotes angiogenesis by modulating endothelial TGF-beta signalling. Nature. 2013; 499:306-11.
54. Li Y Y, Zhang Y, Qiu F, Qiu Z Y. Proteomic identification of exosomal LRG1: a potential urinary biomarker for detecting NSCLC. Electrophoresis. 2011; 32:1976-83.
55. Zhang J J, Zhu L Y, Fang J Y, Ge Z Z, Li X B. LRG1 modulates epithelial-mesenchymal transition and angiogenesis in colorectal cancer via HIF-1alpha activation. J Exp Clin Cancer Res. 2016; 35:29.
56. Sandanayake N S, Sinclair J, Andreola F, Chapman M H, Xue A, Webster G J, et al. A combination of serum leucine-rich alpha-2-glycoprotein 1, CA19-9 and interleukin-6 differentiate biliary tract cancer from benign biliary strictures. Br J Cancer. 2011; 105:1370-8.
57. Li Q K, Chen L, Ao M H, Chiu J H, Zhang Z, Zhang H, et al. Serum fucosylated prostate-specific antigen (PSA) improves the differentiation of aggressive from non-aggressive prostate cancers. Theranostics. 2015; 5:267-76.
58. Wang C, Hoti N, Lih T M, Sokoll L J, Zhang R, Zhang Z, et al. Development of a glycoproteomic strategy to detect more aggressive prostate cancer using lectin-immunoassays for serum fucosylated PSA. Clin Proteomics. 2019; 16:13.

TABLE 2
Candidate biomarkers with lower expression in AG PCa samples (n = 29)
9 glycopeptide signatures with good performance

| | AUC | Specificity | Sensitivity |
|---|---|---|---|
| Marker | (95% CI) | (95% CI) | (95% CI) |
| M1: ACPP(FLN*ESYK) (SEQ ID NO: 1) | 0.73(0.65, 0.81) | 54.7%(43%, 66%) | 85.7%(76%, 92%) |
| M2: Serum PSA | 0.69(0.6, 0.78) | 85.9%(75%, 92%) | 54.3%(43%, 65%) |
| Combined M1 & M2 | 0.82(0.75, 0.89) | 60.9%(49%, 72%) | 88.6%(79%, 94%) |
| M1: CD63(CCGAAN*YTDWEK) (SEQ ID NO: 6) | 0.69(0.55, 0.83) | 70.8%(51%, 85%) | 71.4%(53%, 85%) |
| M2: Serum PSA | 0.76(0.63, 0.89) | 87.5%(69%, 96%) | 67.9%(49%, 82%) |
| Combined M1 & M2 | 0.81(0.69, 0.93) | 95.8%(80%, 99%) | 60.7%(42%, 76%) |
| M1: ATRN(ISN*SSDTVECECSENWK) | 0.69(0.53, 0.85) | 75%(51%, 90%) | 61.5%(43%, 78%) |
| (SEQ ID NO: 7) | | | |
| M2: Serum PSA | 0.74(0.59, 0.89) | 93.8%(72%, 99%) | 65.4%(46%, 81%) |
| Combined M1 & M2 | 0.84(0.72, 0.96) | 81.2%(57%, 93%) | 80.8%(62%, 91%) |
| M1: GP2(QDLN*SSDVHSLQPQLDCGPR) | 0.74(0.6, 0.88) | 76.2%(55%, 89%) | 67.9%(49%, 82%) |
| (SEQ ID NO: 8) | | | |
| M2: Serum PSA | 0.8(0.68, 0.92) | 90.5%(71%, 97%) | 71.4%(53%, 85%) |
| Combined M1 & M2 | 0.85(0.74, 0.96) | 85.7%(65%, 95%) | 78.6%(60%, 90%) |
| M1: KLK11(TATESFPHPGFN*NSLPNK) | 0.75(0.61, 0.89) | 90.5%(71%, 97%) | 60%(41%, 77%) |
| (SEQ ID NO: 9) | | | |
| M2: Serum PSA | 0.71(0.56, 0.86) | 90.5%(71%, 97%) | 56%(37%, 73%) |
| Combined M1 & M2 | 0.78(0.65, 0.91) | 66.7%(45%, 83%) | 84%(65%, 94%) |
| M1: PTPRN2(VSANVQN*VTTEDVEK) | 0.66(0.52, 0.8) | 65.2%(45%, 81%) | 81.1%(66%, 91%) |
| (SEQ ID NO: 10) | | | |
| M2: Serum PSA | 0.75(0.63, 0.87) | 87%(68%, 95%) | 64.9%(49%, 78%) |
| Combined M1 & M2 | 0.82(0.72, 0.92) | 65.2%(45%, 81%) | 86.5%(72%, 94%) |
| M1: NPTN(AN*ATIEVK) (SEQ ID NO: 11) | 0.72(0.55, 0.89) | 72.7%(43%, 90%) | 72%(52%, 86%) |
| M2: Serum PSA | 0.7(0.52, 0.88) | 81.8%(52%, 95%) | 68%(48%, 83%) |
| Combined M1 & M2 | 0.82(0.68, 0.96) | 54.5%(28%, 79%) | 100%(87%, 100%) |
| M1: CPE(DLQGNPIAN*ATISVEGIDHDVTSAK) | 0.73(0.58, 0.88) | 77.8%(55%, 91%) | 68%(48%, 83%) |
| (SEQ ID NO: 12) | | | |
| M2: Serum PSA | 0.71(0.56, 0.86) | 94.4%(74%, 99%) | 60%(41%, 77%) |
| Combined M1 & M2 | 0.82(0.7, 0.94) | 83.3%(61%, 94%) | 76%(57%, 89%) |
| M1: | 0.72(0.57, 0.87) | 70.6%(47%, 87%) | 74.1%(55%, 87%) |
| RNASE2(NQNTFLLTTFANVVNVCGNPN*MTCPSN*K) | | | |
| (SEQ ID NO: 13) | | | |
| M2: Serum PSA | 0.74(0.59, 0.89) | 94.1%(73%, 99%) | 63%(44%, 78%) |
| Combined M1 & M2 | 0.8(0.67, 0.93) | 58.8%(36%, 78%) | 92.6%(77%, 98%) |

TABLE 3
Candidate biomarkers with higher expression in AG PCa samples (n = 25)
11 glycopeptide signatures with good performance

| | AUC | Specificity | Sensitivity |
|---|---|---|---|
| Marker | (95% CI) | (95% CI) | (95% CI) |
| M1: DSC2(NGIYN*ITVLASDQGGR) (SEQ ID NO: 14) | 0.69(0.56, 0.82) | 81%(60%, 92%) | 65.9%(51%, 78%) |
| M2: Serum PSA | 0.72(0.59, 0.85) | 85.7%(65%, 95%) | 63.6%(49%, 76%) |
| Combined M1 & M2 | 0.79(0.68, 0.9) | 71.4%(50%, 86%) | 77.3%(63%, 87%) |
| M1: LOX(AEN*QTAPGEVPALSNLRPPSR) (SEQ ID NO: 2) | 0.64(0.55, 0.73) | 73.8%(62%, 83%) | 58.8%(47%, 70%) |
| M2: Serum PSA | 0.68(0.59, 0.77) | 72.1%(60%, 82%) | 67.6%(56%, 78%) |
| Combined M1 & M2 | 0.73(0.64, 0.82) | 67.2%(55%, 78%) | 75%(64%, 84%) |
| M1: LRG1(LPPGLLAN*FTLLR) (SEQ ID NO: 15) | 0.65(0.52, 0.78) | 66.7%(48%, 81%) | 60.9%(46%, 74%) |
| M2: Serum PSA | 0.74(0.63, 0.85) | 88.9%(72%, 96%) | 60.9%(46%, 74%) |
| Combined M1 & M2 | 0.8(0.7, 0.9) | 70.4%(52%, 84%) | 80.4%(67%, 89%) |
| M1: CLU(EDALN*ETR) (SEQ ID NO: 3) | 0.66(0.57, 0.75) | 35%(24%, 48%) | 91.7%(83%, 96%) |
| M2: Serum PSA | 0.68(0.59, 0.77) | 83.3%(72%, 91%) | 56.9%(45%, 68%) |
| Combined M1 & M2 | 0.72(0.63, 0.81) | 90%(80%, 95%) | 48.6%(37%, 60%) |
| M1: SERPINA1(YLGN*ATAIFFLPDEGK) (SEQ ID NO: 4) | 0.64(0.55, 0.73) | 54.2%(42%, 66%) | 74.6%(63%, 83%) |
| M2: Serum PSA | 0.7(0.61, 0.79) | 86.4%(75%, 93%) | 57.7%(46%, 69%) |
| Combined M1 & M2 | 0.72(0.63, 0.81) | 69.5%(57%, 80%) | 71.8%(60%, 81%) |
| M1: GRN(DVECGEGHFCHDN*QTCCR) (SEQ ID NO: 16) | 0.68(0.5, 0.86) | 50%(25%, 75%) | 88.5%(71%, 96%) |
| M2: Serum PSA | 0.71(0.54, 0.88) | 91.7%(65%, 99%) | 61.5%(43%, 78%) |
| Combined M1 & M2 | 0.77(0.62, 0.92) | 100%(76%, 100%) | 53.8%(35%, 71%) |
| M1: PTGDS(SVVAPATDGGLN*LTSTFLR) (SEQ ID NO: 17) | 0.65(0.56, 0.74) | 45.6%(34%, 57%) | 83.8%(74%, 90%) |
| M2: Serum PSA | 0.69(0.6, 0.78) | 83.8%(73%, 91%) | 56.8%(45%, 67%) |
| Combined M1 & M2 | 0.72(0.64, 0.8) | 73.5%(62%, 83%) | 64.9%(54%, 75%) |
| M1: UMOD(QDFN*ITDISLLEHR) (SEQ ID NO: 18) | 0.64(0.55, 0.73) | 58.8%(47%, 70%) | 71.6%(60%, 81%) |
| M2: Serum PSA | 0.69(0.6, 0.78) | 83.8%(73%, 91%) | 56.8%(45%, 67%) |
| Combined M1 & M2 | 0.7(0.61, 0.79) | 70.6%(59%, 80%) | 64.9%(54%, 75%) |
| M1: AFM(DIENFN*STQK) (SEQ ID NO: 19) | 0.67(0.57, 0.77) | 63.6%(50%, 75%) | 75.8%(64%, 84%) |
| M2: Serum PSA | 0.7(0.61, 0.79) | 80%(68%, 88%) | 62.1%(50%, 73%) |
| Combined M1 & M2 | 0.73(0.64, 0.82) | 67.3%(54%, 78%) | 72.7%(61%, 82%) |
| M1: ORM1(QDQCIYN*TTYLNVQR) (SEQ ID NO: 5) | 0.64(0.55, 0.73) | 38.8%(28%, 51%) | 84.9%(75%, 91%) |
| M2: Serum PSA | 0.7(0.61, 0.79) | 86.6%(76%, 93%) | 56.2%(45%, 67%) |
| Combined M1 & M2 | 0.73(0.65, 0.81) | 79.1%(68%, 87%) | 64.4%(53%, 74%) |
| M1: CD97(WCPQNSSCVN*ATACR) (SEQ ID NO: 21) | 0.68(0.58, 0.78) | 65.5%(52%, 77%) | 71.9%(60%, 81%) |
| M2: Serum PSA | 0.73(0.64, 0.82) | 85.5%(74%, 92%) | 62.5%(50%, 73%) |
| Combined M1 & M2 | 0.78(0.7, 0.86) | 69.1%(56%, 80%) | 81.2%(70%, 89%) |

TABLE 4
Candidate glycoproteomic signatures show good performance in distinguishing AG PCa from NAG PC

[symbol]

[symbol]

indicates data missing or illegible when filed

TABLE 5
ROC analysis of panels of signatures including ACPP (FLN*ESYK), one up-regulated
glycopeptide and serum PSA. The ROC curves for CLU (EDALN*ETR), LOX
(AEN*QTAPGEVPALSNLRPPSR), SERPINA1 (YLGN*ATAIFFLPDEGK) and ORM1
(QDQCIYN*TTYLNVQR) are shown in FIG. 5. The specificity at 95% sensitivity is shown in
the table.

| | | | |
|---|---|---|---|
| | AUC | Speci- | Sensi- |
| Signatures | (95% confidence interval) | ficity | tivity |

Result corresponding to FIG. 5A

| | | | |
|---|---|---|---|
| M1: ACPP(FLN*ESYK) (SEQ ID NO: 1) | 0.73(0.65, 0.81) | 20% | 95% |
| M2: CLU(EDALN*ETR) (SEQ ID NO: 3) | 0.68(0.59, 0.77) | 27% | 95% |
| M3: Serum PSA | 0.69(0.6, 0.78) | 8% | 95% |
| Combined M1 & M2 | 0.8(0.73, 0.87) | 41% | 95% |
| Combined M1 to M3 | 0.86(0.8, 0.92) | 50% | 95% |

Result corresponding to FIG. 5B

| | | | |
|---|---|---|---|
| M1: ACPP(FLN*ESYK) (SEQ ID NO: 1) | 0.73(0.65, 0.81) | 20% | 95% |
| M2: LOX(AEN*QTAPGEVPALSNLRPPSR) (SEQ ID NO: 2) | 0.63(0.54, 0.72) | 14% | 95% |
| M3: Serum PSA | 0.69(0.6, 0.78) | 8% | 95% |
| Combined M1 & M2 | 0.75(0.67, 0.83) | 20% | 95% |
| Combined M1 to M3 | 0.82(0.75, 0.89) | 47% | 95% |

Result corresponding to FIG. 5C

| | | | |
|---|---|---|---|
| M1: ACPP(FLN*ESYK) (SEQ ID NO: 1) | 0.73(0.65, 0.81) | 20% | 95% |
| M2: SERPINA1(YLGN*ATAIFFLPDEGK) (SEQ ID NO: 4) | 0.66(0.57, 0.75) | 9% | 95% |
| M3: Serum PSA | 0.69(0.6, 0.78) | 8% | 95% |
| Combined M1 & M2 | 0.76(0.68, 0.84) | 41% | 95% |
| Combined M1 to M3 | 0.83(0.76, 0.9) | 50% | 95% |

Result corresponding to FIG. 5D

| | | | |
|---|---|---|---|
| M1: ACPP(FLN*ESYK) (SEQ ID NO: 1) | 0.73(0.65, 0.81) | 20% | 95% |
| M2: ORM1(QDQCIYN*TTYLNVQR) (SEQ ID NO :5) | 0.63(0.54, 0.72) | 14% | 95% |
| M3: Serum PSA | 0.69(0.6, 0.78) | 8% | 95% |
| Combined M1 & M2 | 0.76(0.68, 0.84) | 20% | 95% |
| Combined M1 to M3 | 0.83(0.76, 0.9) | 50% | 95% |

TABLE 6
ROC analysis of panels of signatures including ACPP (FLN*ESYK), one up-regulated
glycopeptide and serum PSA. The specificity at 95% sensitivity is shown in the table.

| | AUC | Speci- | Sensi- |
|---|---|---|---|
| Signatures | (95% confidence interval) | ficity | tivity |
| M1: ACPP(FLN*ESYK) (SEQ ID NO: 1) | 0.73(0.65, 0.81) | 20% | 95% |
| M2: PTGDS(SVVAPATDGGLN*LTSTFLR) | 0.6(0.5, 0.7) | 5% | 95% |
| (SEQ ID NO: 17) | | | |
| M3: Serum PSA | 0.69(0.6, 0.78) | 8% | 95% |
| Combined M1 & M2 | 0.77(0.69, 0.85) | 25% | 95% |
| Combined M1 to M3 | 0.83(0.76, 0.9) | 34% | 95% |
| M1: ACPP(FLN*ESYK) (SEQ ID NO: 1) | 0.73(0.65, 0.81) | 20% | 95% |
| M2: GRN(DVECGEGHFCHDN*QTCCR) | 0.56(0.46, 0.66) | 2% | 95% |
| (SEQ ID NO: 16) | | | |
| M3: Serum PSA | 0.69(0.6, 0.78) | 8% | 95% |
| Combined M1 & M2 | 0.74(0.66, 0.82) | 28% | 95% |
| Combined M1 to M3 | 0.85(0.78, 0.92) | 38% | 95% |
| M1: ACPP(FLN*ESYK) (SEQ ID NO: 1) | 0.73(0.65, 0.81) | 20% | 95% |
| M2: AFM(DIENFN*STQK) (SEQ ID NO: 19) | 0.67(0.58, 0.76) | 3% | 95% |
| M3: Serum PSA | 0.69(0.6, 0.78) | 8% | 95% |
| Combined M1 & M2 | 0.77(0.69, 0.85) | 27% | 95% |
| Combined M1 to M3 | 0.84(0.77, 0.91) | 40.0% | 95% |
| M1: ACPP(FLN*ESYK) (SEQ ID NO: 1) | 0.73(0.65, 0.81) | 20% | 95% |
| M2: UMOD(QDFN*ITDISLLEHR) | 0.61(0.52, 0.7) | 14% | 95% |
| (SEQ ID NO: 18) | | | |
| M3: Serum PSA | 0.69(0.6, 0.78) | 8% | 95% |
| Combined M1 & M2 | 0.75(0.67, 0.83) | 23% | 95% |
| Combined M1 to M3 | 0.81(0.74, 0.88) | 44% | 95% |
| M1: ACPP(FLN*ESYK) (SEQ ID NO: 1) | 0.73(0.65, 0.81) | 20% | 95% |
| M2: LRG1(LPPGLLAN*FTLLR) | 0.61(0.52, 0.7) | 5% | 95% |
| (SEQ ID NO: 15) | | | |
| M3: Serum PSA | 0.69(0.6, 0.78) | 8% | 95% |
| Combined M1 & M2 | 0.76(0.68, 0.84) | 28% | 95% |
| Combined M1 to M3 | 0.85(0.78, 0.92) | 36% | 95% |
| M1: ACPP(FLN*ESYK) (SEQ ID NO: 1) | 0.73(0.65, 0.81) | 20% | 95% |
| M2: DSC2(NGIYN*ITVLASDQGGR) | 0.65(0.56, 0.74) | NA | 95% |
| (SEQ ID NO: 14) | | | |
| M3: Serum PSA | 0.69(0.6, 0.78) | 8% | 95% |
| Combined M1 & M2 | 0.76(0.68, 0.84) | 30% | 95% |
| Combined M1 to M3 | 0.84(0.77, 0.91) | 42% | 95% |
| M1: ACPP(FLN*ESYK) (SEQ ID NO: 1) | 0.73(0.65, 0.81) | 20% | 95% |
| M2: CD97(WCPQNSSCVN*ATACR) | 0.65(0.56, 0.74) | 6% | 95% |
| (SEQ ID NO: 21) | | | |
| M3: Serum PSA | 0.69(0.6, 0.78) | 8% | 95% |
| Combined M1 & M2 | 0.76(0.68, 0.84) | 27% | 95% |
| Combined M1 to M3 | 0.84(0.77, 0.91) | 41% | 95% |

TABLE 7
Comparison of the quantification results of glycopeptides across the discovery set, validation set 1 and validation set 2.
The 13 glycopeptides show the same trend between the discovery set and validation set are included here.
Discovery Cohort
142 samples (74 AG and 68 NAG)

| | | | | | log2 Fold | Fold | Num. | Num. | Higher |
|---|---|---|---|---|---|---|---|---|---|
| Glycopeptides | Gene | UniProt | Protein Description | p-value | Change | Change | of NAG | of AG | Expression |
| FLN*ESYK (SEQ ID NO: 1) | ACPP | P15309 | Prostatic acid phosphatase | 1.94E-06 | −1.35734 | 2.562118 | 64 | 70 | NAG |
| NN*HTASILDR | CD63 | P08962 | CD63 antigen | 0.044674 | −0.59545 | 1.510948 | 31 | 42 | NAG |
| (SEQ ID NO: 22) | | | | | | | | | |
| QDLN*SSDVHSLQPQLDCGPR | GP2 | P55259 | Pancreatic secretory granule | 0.001617 | −1.23753 | 2.357948 | 21 | 28 | NAG |
| (SEQ ID NO: 8) | | | membrane major glycoprotein | | | | | | |
| | | | GP2 | | | | | | |
| NQNTFLLTTFANVVNVCGNPN*M | RNASE2 | P10153 | Non-secretory ribonuclease | 0.005118 | −1.36107 | 2.568765 | 17 | 27 | NAC |
| TCPSN*K (SEQ ID NO: 13) | | | | | | | | | |
| NGIYN*ITVLASDQGGR | DSC2 | Q02487 | Desmocollin-2 | 0.008142 | 0.694569 | 1.618401 | 21 | 44 | AG |
| (SEQ ID NO: 14) | | | | | | | | | |
| EDALN*ETR | CLU | P10909 | Clusterin | 0.001243 | 0.715886 | 1.642492 | 60 | 72 | AG |
| (SEQ ID NO: 3) | | | | | | | | | |
| YLGN*ATAIFFLPDEGK | SERPINA1 | P01009 | Alpha-1-antitrypsin | 0.001404 | 0.873164 | 1.831675 | 59 | 71 | AG |
| (SEQ ID NO: 4) | | | | | | | | | |
| DVECGEGHFCHDN*QTCCR | GRN | P28799 | Progranulin | 0.019442 | 1.64495 | 3.127371 | 12 | 26 | AG |
| (SEQ ID NO: 16) | | | | | | | | | |
| SVVAPATDGGLN*LTSTFLR | PTGDS | P41222 | Prostaglandin-H2 D-isomerase | 0.0013 | 0.91689 | 1.888041 | 68 | 74 | AG |
| (SEQ ID NO: 17) | | | | | | | | | |
| QDFN*ITDISLLEHR | UMOD | P07911 | Uromodulin | 0.00035 | 1.633053 | 3.101687 | 68 | 74 | AG |
| (SEQ ID NO: 18) | | | | | | | | | |
| AEN*QTAPGEVPALSNLRPPSR | LOX | P28300 | Protein-lysine 6-oxidase | 0.001987 | 0.700829 | 1.625438 | 61 | 68 | AG |
| (SEQ ID NO: 2) | | | | | | | | | |
| QDQCIYN*TTYLNVQR | ORM1 | P02763 | Alpha-1-acid glycoprotein 1 | 0.000728 | 1.339568 | 2.530755 | 67 | 73 | AG |
| (SEQ ID NO: 5) | | | | | | | | | |
| WCPQNSSCVN*ATACR | CD97 | P48960 | CD97 antigen | 0.000243 | 0.612184 | 1.528571 | 55 | 64 | AG |
| (SEQ ID NO: 21) | | | | | | | | | |

TABLE 8
Comparison of the quantification results of glycopeptides across the discovery set, validation set 1 and validation set 2.
The 13 glycopeptides show the same trend between the discovery set and validation set are included here.
Validation Cohort
The 13 glycopeptides show the same trend between the discovery set and Validation Cohort

| | | | | | | | | |
|---|---|---|---|---|---|---|---|---|
| | | | log2 Fold | | Num. | Num. | Higher | Same trend |
| Glycopeptides | Gene | p-value | Change | Fold Change | of NAG | of AG | expression in | as discovery |

40 AG and 37 NAG (validation set 1)

| | | | | | | | | |
|---|---|---|---|---|---|---|---|---|
| FLN*ESYK (SEQ ID NO: 1) | ACPP | 2.44E-05 | −1.430561 | 2.695514294 | 37 | 40 | NAG | Yes |
| CCGAAN*YTDWEK (SEQ ID NO: 6) | CD63 | 0.151044 | -0.363094 | 1.286181014 | 31 | 35 | NAG | Yes |
| QDLN*SSDVHSLQPQLDCGPR (SEQ ID NO: 8) | GP2 | 0.918817 | −0.160482 | 1.117660539 | 15 | 11 | NAG | Yes |
| NQNTFLLTTFANVVNVCGNPN*MTCPSN*K | RNASE2 | 0.482564 | −0.309658 | 1.239413946 | 13 | 17 | NAG | Yes |
| (SEQ ID NO: 13) | | | | | | | | |
| NGIYN*ITVLASDQGGR (SEQ ID NO: 14) | DSC2 | 0.306347 | 1.2204474 | 2.33018964 | 37 | 37 | AG | Yes |
| EDALN*ETRESETK (SEQ ID NO: 3) | CLU | 0.216772 | 0.1448954 | 1.105650483 | 37 | 40 | AG | Yes |
| YLGN*ATAIFFLPDEGK (SEQ ID NO: 4) | SERPINA1 | 0.79603 | 0.1458275 | 1.106365049 | 37 | 40 | AG | Yes |
| DVECGEGHFCHDN*QTCCR (SEQ ID NO: 16) | GRN | 0.544506 | 0.1384111 | 1.100692188 | 23 | 26 | AG | Yes |
| SVVAPATDGGLN*LTSTFLR (SEQ ID NO: 17) | PTGDS | 0.327126 | 0.3437159 | 1.26902095 | 37 | 40 | AG | Yes |
| QDFN*ITDISLLEHR (SEQ ID NO: 18) | UMOD | 0.070935 | 0.8484202 | 1.800528209 | 36 | 40 | AG | Yes |
| AEN*QTAPGEVPALSNLRPPSR (SEQ ID NO: 2) | LOX | 0.020481 | 0.6907688 | 1.614143447 | 27 | 33 | AG | Yes |
| QDQCIYN*TTYLNVQR (SEQ ID NO: 5) | ORMI | 0.379919 | 0.2452057 | 1.185261756 | 37 | 40 | AG | Yes |
| WCPQNSSCVN*ATACR (SEQ ID NO: 21) | CD97 | 0.087313 | 0.2856123 | 1.218927494 | 31 | 40 | AG | Yes |

40 AG and 13 NAG (validation set 2)

| | | | | | | | | |
|---|---|---|---|---|---|---|---|---|
| FLN*ESYK (SEQ ID NO: 1) | ACPP | 0.021032 | −1.197453 | 2.293344383 | 13 | 40 | NAG | Yes |
| CCGAAN*YTDWEK (SEQ ID NO: 6) | CD63 | 0.259183 | −0.31503 | 1.244037491 | 13 | 35 | NAG | Yes |
| QDLN*SSDVHSLQPQLDCGPR (SEQ ID NO: 8) | GP2 | 0.599878 | 0.373201 | 1.295223871 | 8 | 11 | AG | No |
| NQNTFLLTTFANVVNVCGNPN*MTCPSN*K | RNASE2 | 0.418071 | −0.475012 | 1.389930087 | 7 | 17 | NAG | Yes |
| (SEQ ID NO: 13) | | | | | | | | |
| NGIYN*ITVLASDQGGR (SEQ ID NO: 14) | DSC2 | 0.930395 | 0.003133 | 1.002174104 | 13 | 37 | Unchanged | No |
| EDALN*ETRESETK (SEQ ID NO: 3) | CLU | 0.645741 | −0.026983 | 1.018879521 | 13 | 40 | Unchanged | No |
| YLGN*ATAIFFLPDEGK (SEQ ID NO: 4) | SERPINA1 | 0.493244 | −0.118221 | 1.085395349 | 13 | 40 | Unchanged | No |
| DVECGEGHFCHDN*QTCCR (SEQ ID NO: 16) | GRN | 0.962973 | −0.011415 | 1.007943379 | 12 | 26 | Unchanged | No |
| SVVAPATDGGLN*LTSTFLR (SEQ ID NO: 17) | PTGDS | 0.878312 | −0.057353 | 1.040554909 | 13 | 40 | Unchanged | No |
| QDFN*ITDISLLEHR (SEQ ID NO: 18) | UMOD | 0.910605 | −0.089562 | 1.064046768 | 13 | 40 | Unchanged | No |
| AEN*QTAPGEVPALSNLRPPSR (SEQ ID NO: 2) | LOX | 0.051278 | 0.889931 | 1.853087445 | 10 | 33 | AG | Yes |
| QDQCIYN*TTYLNVQR (SEQ ID NO: 5) | ORM1 | 0.532896 | 0.281266 | 1.215260971 | 13 | 40 | AG | Yes |
| WCPQNSSCVN*ATACR (SEQ ID NO: 21) | CD97 | 0.206306 | 0.257049 | 1.195031825 | 13 | 40 | AG | Yes |

TABLE 9
Validation results of glycopeptide from ACPP, CD63, DSC2, LOX and LRG1 as
compared to their results in discovery cohort.

| | | Validation cohort | |
|---|---|---|---|
| | | 40 AG and 37 NAG | 40 AG and 13 NAG |
| | Discovery cohort | (validation set 1) | (validation set 2) |
| | AUC | AUC | AUC |
| | (95% confidence | (95% confidence | (95% confidence |
| Signatures | interval) | interval) | interval) |
| M1: ACPP(FLN*ESYK) | 0.73(0.65, 0.81) | 0.77(0.67, 0.88) | 0.71(0.56, 0.86) |
| (SEQ ID NO: 1) | | | |
| M2: Serum PSA | 0.69(0.6, 0.78) | 0.8(0.69,0.9) | 0.81(0.68, 0.94) |
| Combined M1 & M2 | 0.82(0.75, 0.89) | 0.83(0.74, 0.92) | 0.8(0.67, 0.93) |
| M1: CD63(CCGAAN*YTDWEK) | 0.69(0.55, 0.83) | 0.6(0.46, 0.74) | 0.61(0.43, 0.78) |
| (SEQ ID NO: 6) | | | |
| M2: Serum PSA | 0.76(0.63, 0.89) | 0.77(0.65, 0.89) | 0.79(0.65, 0.94) |
| Combined M1 & M2 | 0.81(0.69, 0.93) | 0.79(0.68, 0.9) | 0.81(0.69, 0.94) |
| M1: DSC2(NGIYN*ITVLASDQGGR) | 0.69(0.56, 0.82) | 0.57(0.44, 0.7) | 0.51(0.32, 0.7) |
| (SEQ ID NO: 14) | | | |
| M2: Serum PSA | 0.72(0.59, 0.85) | 0.79(0.68, 0.89) | 0.8(0.65, 0.94) |
| Combined M1 & M2 | 0.79(0.68, 0.9) | 0.77(0.66, 0.88) | 0.73(0.57, 0.88) |
| M1: LOX | 0.64(0.55, 0.73) | 0.67(0.54, 0.81) | 0.71(0.51, 0.9) |
| (AEN*QTAPGEVPALSNLRPPSR) | | | |
| (SEQ ID NO: 2) | | | |
| M2: Serum PSA | 0.68(0.59, 0.77) | 0.76(0.64, 0.89) | 0.78(0.62, 0.95) |
| Combined M1 & M2 | 0.73(0.64, 0.82) | 0.8(0.68, 0.91) | 0.81(0.67, 0.95) |
| M1: LRG1(LPPGLLAN*FTLLR) | 0.65(0.52, 0.78) | 0.53(0.38, 0.67) | 0.53(0.34, 0.72) |
| (SEQ ID NO: 15) | | | |
| M2: Serum PSA | 0.74(0.63, 0.85) | 0.79(0.67, 0.91) | 0.8(0.66, 0.94) |
| Combined M1 & M2 | 0.8(0.7, 0.9) | 0.78(0.66, 0.89) | 0.8(0.67, 0.93) |

TABLE 10
Validation results of the panels of signatures.
40 AG and 37 NAG
(Validation set 1)

| | AUC | | |
|---|---|---|---|
| Panel of candidate biomarkers | (95% confidence interval) | Specificity | Sensitivity |
| ACPP(FLN*ESYK) (SEQ NO: 1)& CLU(EDALN*ETR) (SEQ NO: 3) & Serum PSA | 0.85(0.76, 0.94) | 65% | 95% |
| ACPP(FLN*ESYK) (SEQ NO: 1) & LOX(AEN*QTAPGEVPALSNLRPPSR) (SEQ NO: 2) | 0.85(0.76, 0.93) | 54% | 95% |
| & Serum PSA | | | |
| ACPP(FLN*ESYK) (SEQ NO: 1) & SERPINA1(YLGN*ATAIFFLPDEGK) (SEQ NO: 4) | 0.84(0.75, 0.93) | 51% | 95% |
| & Serum PSA | | | |
| ACPP(FLN*ESYK) (SEQ NO: 1) & ORM1(QDQCIYN*TTYLNVQR) (SEQ NO: 20) | 0.82(0.72, 0.91) | 54% | 95% |
| & Serum PSA | | | |

40 AG and 13 NAG
(Validation set 2)

| | AUC | | |
|---|---|---|---|
| Panel of candidate biomarkers | (95% confidence interval) | Specificity | Sensitivity |
| ACPP(FLN*ESYK) (SEQ NO: 1) & CLU(EDALN*ETR) (SEQ NO: 3) & Serum PSA | 0.76(0.6, 0.92) | 46% | 95% |
| ACPP(FLN*ESYK) (SEQ NO: 1) & LOX(AEN*QTAPGEVPALSNLRPPSR) (SEQ NO: 2) | 0.81(0.69, 0.93) | 38% | 95% |
| & Serum PSA | | | |
| ACPP(FLN*ESYK) (SEQ NO: 1) & SERPINA1(YLGN*ATAIFFLPDEGK) (SEQ NO: 4) & Serum PSA | 0.82(0.7, 0.94) | 38% | 95% |
| ACPP(FLN*ESYK) (SEQ NO: 1) & ORM1(QDQCIYN*TTYLNVQR) (SEQ NO: 20) & Serum PSA | 0.82(0.71, 0.94) | 38% | 95% |

### Example 2: Development of Parallel Reaction Monitoring (PRM) Assays for the Detection of Aggressive Prostate Cancer Using Urinary Glycoproteins

Recently, the present inventors have found two urinary glycoproteins, Prostatic Acid Phosphatase (ACPP) and Clusterin (CLU) combined with serum prostate-specific antigen (PSA) can serve as a three-signature panel for detecting aggressive prostate cancer (PCa) based on a quantitative glycoproteomic study. To facilitate the translation of candidates into clinically applicable tests, robust and accurate targeted parallel reaction monitoring (PRM) assays that can be widely adopted in multi-labs were developed in this study. The developed PRM assays for the urinary glycopeptides, FLN*ESYK (SEQ ID NO:1) from ACPP and EDALN*ETR (SEQ ID NO:3) from CLU, demonstrated good repeatability and sufficient working range covering three to four orders of magnitude, and their performances in differentiating the aggressive prostate cancer were assessed by the quantitative analysis of urine specimens collected from 69 non-aggressive (Gleason score=6) and 73 aggressive (Gleason≥8) PCa patients. When ACPP combined with CLU, the discrimination power was improved from an AUC of 0.66 to 0.78. By combining ACPP, CLU and serum PSA to form a three-signature panel, the AUC was further improved to 0.83 (sensitivity: 84.9%, specificity: 68.1%). Since the serum PSA test alone had an AUC of 0.68, the present inventors' results demonstrated that the new urinary glycopeptide PRM assays can serve as an adjunct to the serum PSA test to achieve better predictive power toward aggressive PCa. In summary, the present inventors' developed PRM assays for urinary glycopeptides were successfully applied to clinical PCa urine samples with a promising performance in aggressive PCa detection.

### Introduction

Prostate cancer (PCa) is the most common diagnosed male malignancy and the second-leading cause of death for men in developed countries¹. Most PCa is indolent at the time of diagnosis. The indolent PCa is slow growing and poses limited threat to patients' life even without therapy intervention applied. Once the PCa is developed into a more advanced stage, it will progress rapidly increasing mortality rate of patients; thus, systematic and intensified therapy interventions will be required^{2,3}. Currently, the clinical characterization of the aggressiveness of the PCa is mainly assessed by serum prostate-specific antigen (PSA) testing, digital rectal examination (DRE), repeated prostate tissue needle biopsies to derive the Gleason score and TNM staging system⁴. Due to the low specificity of serum PSA testing aggressiveness of PCa as well as the pain and complications caused by the invasive tissue biopsy, it is crucial to identify non-invasive biomarkers associated with aggressive PCa and implement the biomarkers into robust clinical testing methods to guide PCa risk stratification.

Urine is an appealing substrate for the discovery of non-invasive biomarkers associated with PCa, since urinary system is proximal to the prostate gland and may contain molecular signatures shed or secreted from diseased prostate tissue, such as tumor cells, DNA/RNA and proteins⁵. Indeed, isolation of circulating tumor cells from urine of PCa patients was achieved by the microfluid chip strategy and the amount of tumor cells displayed moderate correlation to the Gleason score⁶. Great efforts have also been directed toward the investigation of urine-derived genetic biomarkers including long non-coding RNA, micro-RNA, DNA, and gene fusion. For example, prostate cancer antigen-3 (PCA3) is the first urinary biomarker approved by US Food and Drug Administration (FDA) for PCa; assessments of its individual performance as well as in combination with urinary TMPRSS2:ERG gene fusions as diagnostic or prognostic biomarkers for PCa were conducted in multiple studies⁷⁻¹². Additionally, a urine exosome derived gene expression assay was used to predict high-grade PCa at the initial biopsy, which displayed a good discrimination power in differentiating Gleason 6 group from Gleason≥7 group¹³. A previous study reported a three-gene panel (HOXC6, TDRD1, and DLX1) selected from urinary sediments for the early detection of PCa with biopsy Gleason≥7¹⁴. Proteomic signatures obtained from urine specimens were also investigated for their ability in distinguishing aggressive PCa^{15,16}. Although many urinary candidate biomarkers were proposed for aggressive PCa detection, only a few of them entered clinical trials; many candidates still require to further validate their clinical utility^{5,17}.

In the present inventors' previous study, an automated high-throughput urine sample preparation platform was coupled with data-independent acquisition (DIA) of mass spectrometry (MS) to discover urinary glycoproteins associated with aggressive PCa, of which glycopeptides from the urinary glycoproteins, Prostatic Acid Phosphatase (ACPP) and Clusterin (CLU), demonstrated promising results¹⁸. Serum ACPP, instead of urinary ACPP, was once served as the world's first clinically useful biomarker for PCa until it was replaced by serum PSA test as the latter demonstrated improved predictive power for early stage pca^{19,20}. Nonetheless, the present inventors' recent studies have suggested the emerging roles of ACPP as a potential prognostic biomarker in detecting aggressive PCa¹⁸. ACPP is a prostate specific protein and its abundance is fifty times higher in prostate tissue than tissues from other organs in human²¹; hence, its abundance variation in different urine specimens may reflect the secretory function of the prostate gland or the progression of PCa. Therefore, FLN*ESYK (SEQ ID NO: 1) (an N-linked glycopeptide, * indicates the glycosite) from ACPP identified via the present inventors' DIA-based approach is worth further evaluation by targeted proteomics. Besides ACPP, glycopeptide EDALN*ETR (SEQ ID NO:3) from CLU showed association with aggressive PCa via DIA. CLU is a glycoprotein involved in diverse biological events including cell proliferation and cell death; it is also associated with tumor progression and neurodegenerative disorders²². CLU represents as one of the multi-functional proteins whose expression is altered in both inflammation and cancer²². Although the investigation of the specific role of CLU in PCa is still ongoing^{23,24}, the up-regulated glycopeptide from CLU discovered in the present inventors' previous study still worth further explored using a targeted approach.

To develop accurate and easily extendible quantitative strategy which can be applied to large-scale clinical cohorts as well as in different laboratories^{25,26}, in this study, targeted parallel reaction monitoring (PRM) assays were established for the two glycopeptides from ACPP and CLU, which were discovered in the present inventors' previous work using DIA-MS approach¹⁸. The developed PRM assays were subsequently applied to the first cohort consisted of 142 PCa urine samples to examine the performance of candidate glycopeptide assays in distinguishing aggressiveness of PCa by receiver operating characteristic (ROC) analysis in repeated 10-fold cross validation with logistic regression. Finally, the PRM assays were further evaluated for their capability in supplementing each other and serum PSA test to achieve a better discrimination power towards aggressive PCa detection. The results indicated that accurate and robust PRM assays were successfully developed for urinary glycopeptides and they can provide additional predictive value for the diagnosis of aggressive PCa relative to serum PSA test.

### Materials and Methods

Urine samples. Post-DRE raw urine specimens from PCa patients of first cohort (69 of Gleason score=6, 73 of Gleason score≥8) and second cohort (13 of Gleason score=6, of Gleason score=8, used for validation) were collected and processed by the Department of Urology at Johns Hopkins University School of Medicine following the EDRN's PCA3 urine processing standard operating procedure (SOP). The collected urine samples were labeled with specimen identification number and stored at −80° C. freezer. The information of the urine samples and the associated prostate cancer patients were obtained with approval from the Institutional Review Board of Johns Hopkins University and informed consent. The first cohort was from the same set of samples used in the present inventors' previous DIA discovery study¹⁸ to demonstrate the possibility of translating DIA MS-discovered candidates into PRM assays. However, the experimental design, sample preparation, data acquisition, and data analysis were independently from the previous published work. Detailed information on the urine specimens is listed in Supplemental Table S1 (not shown).

Database search and PRM assay evaluation. PRM raw files were analyzed by Skyline (version 20.1.0.76) for peak integration and quantification of the targeted glycopeptides. All peaks were inspected manually to ensure the correct detection of precursor and fragment ions. The heavy (H) and light (L) peptide transition, retention time and peak boundary were also used to confirm peptide identity. A minimum of four transitions was required for the correct detection of the target peptides. The MS responses of endogenous glycopeptides (i.e., light peptides) identified in each urine sample were normalized to their corresponding heavy isotope-labeled internal standards for relative quantification. The ratio of L/H were exported for further statistical analysis.

The calibration standards were prepared using glycopeptides isolated from pooled PCa urine samples as background to mimic the condition of real clinical samples as well as to control matrix effects. To determine the linear dynamic range of MS response, a 12-point dilution series of heavy isotope-labeled standard peptides (5, 2.5, 1, 0.5, 0.25, 0.1, 0.05, 0.025, 0.01, 0.005, 0.001, and 0.0005 pmol per injection) were spiked into the PCa urine derived glycopeptides background (from 50 ul of urine sample per injection) and were measured by PRM in triplicate for their MS response. Due to the wide dynamic range of the abundance of the present inventors' candidate glycopeptides, a 1/x² weighting was performed when generating the linear regression of the calibration curves.

The limit of detection (LOD) and limit of quantitation (LOQ) of the targeted glycopeptides were determined based on the standard deviation of the linear regression curves using the following formula²⁷, LOD=3.3*Sa/b, LOQ=10*Sa/b, where Sa is the standard deviation of the y-intercepts and b represents the slope of the calibration curves.

Statistical Analysis. All the analyses were carried out in R (version 3.5). The expression fold change of the targeted glycopeptides between aggressive and non-aggressive PCa groups were computed and the p-values were calculated using Mann-Whitney U test. For each glycopeptide, its discriminatory power as an individual marker or in combination with others through logistic regression was evaluated by ROC analysis in two repeated 10-fold cross validations. The mean ROC curves from the repeated 10-fold cross validations were depicted and area under a curve (AUC) was computed for the mean ROC curves. The predictive models with cross validation were built using caret²⁸ (version 6.0-85). ROC curves were generated using pROC²⁹ (version 1.13) whereas AUC along with 95% confidence interval (95% CI) as well as sensitivity and specificity at the best cutoff point along with 95% CI were obtained via MLeval (https://CRAN.R-project.org/package=MLeval). The best cutoff point on ROC curve generated the maximal summed sensitivity and specificity. The generated predictive models were further investigated using the second cohort.

Data availability. The LC-MS/MS data have been deposited to the PeptideAtlas repository³⁵ with the dataset identifier: PASS01676.

Chemicals and Reagents. Oasis MAX resins and Sep-Pak C18 resins were purchased from Waters (Milford, MA) and C4 resin beads (35 μm, 300 Å) were from Separation Methods Technologies (Newark, DE). Sequencing-grade trypsin and Lys-C were acquired from Promega (Madison, WI). Enzyme PNGase F was from New England Biolabs (Ipswich, MA). All the other chemicals including urea, ammonia bicarbonate (AB), acetonitrile (ACN), trifluoroacetic acid (TFA), formic acid (FA), triethyl ammonium bicarbonate (TEAB), tris (2-carboxyethyl) phosphine (TCEP), iodoacetamide, and triethylammonium acetate were purchased from Sigma Aldrich (St. Louis, MO). Heavy isotope-labeled peptides with the C-terminal Arg or Lys labeled with N¹⁵ and C¹³ with more than 95% purity were synthesized by Synpeptide Co. (Shanghai, China).

Automated tryptic digestion of human urine samples. For each urine specimen, 500 μL was desalted and tryptic digested automatically on Versette (Thermo Scientific, Waltham, MA) according to the experiment workflow that we published previously (36). First, C4-tips were fabricated with 30 mg of C4 resin beads packed into each tip and conditioned with 50% ACN containing 0.1% TFA followed by 0.1% TFA (10 cycles each). Next, urine samples (500 μL) were acidified (pH<3) and centrifuged at 1,3800 g for 5 minutes. The supernatant was then loaded onto the C4-tips (90 aspiration/dispense cycles). Each aspiration/dispense cycle was performed in approximately two minutes at room temperature. The tips were then rinsed with 0.1% TFA followed by 100 mM triethyl ammonium bicarbonate (TEAB) to remove unbound and contaminant material (10 cycles each). Proteins binding onto the C4-tips were reduced with 10 mM Tris 2-carboxyethyl phosphine (TCEP) in 50 mM TEAB buffer (pH 8.2) at room temperature and alkylated with 15 mM iodoacetamide in the dark (20 cycles each). Proteins were digested in sequence by Lys-C (1:40 enzyme/protein, 30 cycles) and trypsin (1:40 enzyme/protein, 120 cycles) in 50 mM TEAB buffer containing 30% ACN to directly recover digested peptides from C4-tips to solution. The C4-tips were subsequently rinsed twice with 50% ACN containing 0.1% TFA to elute the remaining digested peptides into the solution. Peptide mixtures were dried down and stored at −20° C. until analyzed.

Isolation of N-linked glycosite-containing peptide from human urine samples. An automated strategy recently established by our group were used to enrich the intact glycopeptides from the peptide mixture for each urine sample (37). Firstly, 6 mg Oasis MAX resins and 20 mg C18 resins were stacked into tips to generate the mix-mode enrichment tip. Then the tips were sequentially conditioned by 100% ACN, 100 mM Triethylammonium Acetate (TAAB), 95% ACN containing 1% TFA, and 0.1% TFA (20 cycles each). Secondly, peptide mixtures dissolving in 0.1% TFA were put in 96-well plate and loaded onto the MAX/C18 tips with 15 cycles of aspirating/dispensing followed by a rinse with 0.1% TFA (10 cycles). Peptides were desalted via binding onto C18. Next, desalted intact glycopeptides were eluted from C18 to MAX using 95% ACN/1% TFA. Finally, the bound intact glycopeptides were eluted from MAX by 50% ACN/0.1% TFA and dried down. For the removal of N-glycans, intact glycopeptides were dissolved in 100 mM Tris-HCl at pH 8.0 with 2 μL of PNGase F. The mixture was incubated in 37° C. overnight and subjected to C18-cleanup via StageTip method (38). The generated N-linked glycosite-containing peptides (one tenth of the total glycopeptides enriched from 500 μL urine) were subjected to LC-MS/MS analysis in PRM mode on Q-Exactive HF-X mass spectrometer.

LC-MS/MS analysis. The PRM LC-MS/MS analysis was performed on a Q-Exactive HF-X mass spectrometer connected to an EASY-nLC 1200 system (Thermo Fisher Scientific). A self-packed 28 cm long C18 column (1.9 μm/120 Å ReproSil-Pur C18 resin, Dr. Maisch GmbH, Germany) with an integrated PicoFrit emitter (New Objective) were used for the reversed phase analysis of the peptides. Buffer A: 3% ACN in 0.1% formic acid and buffer B: 90% ACN in 0.1% formic acid were used to generate the binary phase gradient. The gradient used was: 5% to 40% buffer B (80% ACN and 0.1% formic acid) lasted for 88 minutes at a flow rate of 300 nL/minute. Targeted precursor m/z and retention time of the glycopeptides were obtained from previously built spectral library¹⁷ and were assigned to the inclusion list. The synthesized heavy isotope-labeled peptides (with the C-terminal K/R labeled with C¹³ and N¹⁵) corresponding to the candidate glycopeptides were added to the sample as internal standards. Full scan was acquired from m/z 400 to 2000 at a resolution of 60,000, the automatic gain control (AGC) was set as 3×10⁶ with a max injection time of 60 ms. The full MS scan was followed by 20 MS2 scans based on the precursor list. The precursor ions were fragmented by higher-energy collisional dissociation (HCD) to generate MS2 scans. The parameters for MS2 scans were: resolution of 30,000, isolation width of 0.7 m/z; normalized collision energy (NCE) of 30, and dynamic exclusion time of 20 s.

### Results

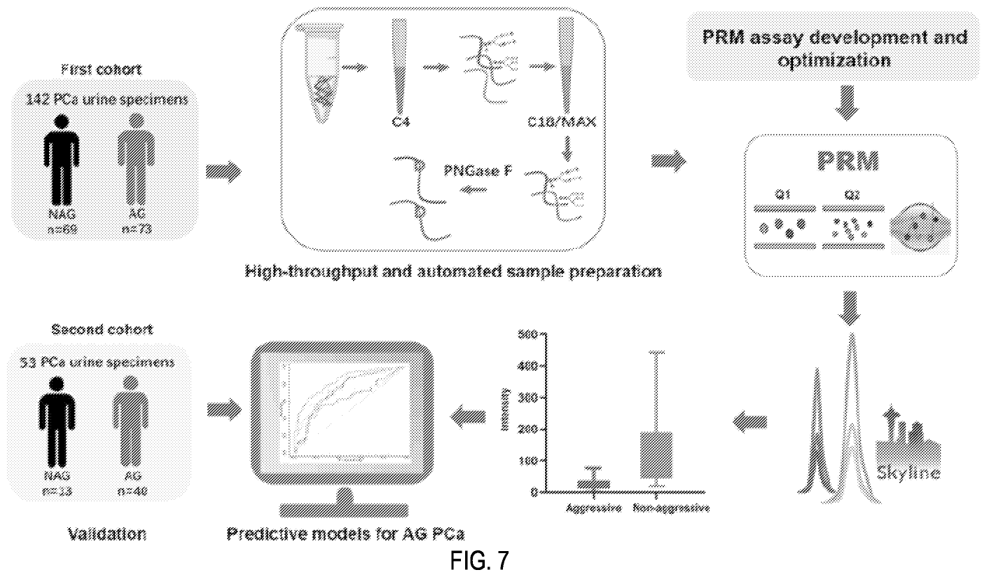

Overview of the experimental workflow. In the present inventors' recent study, two glycopeptides from urinary protein ACPP and CLU were discovered with promising results in detecting aggressive PCa based on quantitative DIA analysis¹⁸. To evaluate the clinical utility of the candidate glycopeptides and facilitate the translation of the MS-based candidate biomarkers to routine clinical implementation in future, the present inventors developed easily extendable PRM quantitative assays for the aforementioned urinary glycopeptides. The present inventors also applied the assays to clinical urine specimens, which were quantitatively analyzed in the present inventors' previous discovery study using DIA to assess the performance of the candidates in separating aggressive PCa from non-aggressive PCa. The schematic workflow of this study is illustrated in FIG. 7. In brief, urine specimens were processed using an automated high-throughput platform to isolate glycopeptides^{30,31}. The PRM assays were developed for the two candidate glycopeptides. The analytical performances of the PRM assays were assessed using heavy-isotope labeled synthetic peptides spiked in the pooled urinary glycopeptides. The enriched urinary glycopeptides from each urine sample were subjected to targeted quantitative LC-MS/MS analysis using the PRM assays. Finally, the relative quantification results of targeted glycopeptides were statistically analyzed to evaluate the clinical performance of PRM assays in combination with serum PSA for discrimination power toward aggressive PCa.

Establishment and characterization of the PRM assays. To develop a targeted PRM assay with high accuracy and high repeatability, it is essential to establish a linear relationship between the measured MS signal response (i.e., intensity) of a targeted peptide and its quantity in the relevant biological matrix background³² (e.g., serum or urine). The measured intensity can reflect true abundance of the targeted peptide if only the MS signal response falls into the established linear detection range. Therefore, the present inventors constructed the reversed calibration curves for the present inventors' candidate glycopeptides by spiking in a serial dilution of heavy isotope-labeled standard peptides to generate 12-point linear curves, spanning from 0.5 fmol/injection to 5,000 fmol/injection, into urinary glycopeptides as the sample matrix background to assess the linear relationship between the MS signals of targeted glycopeptides and their quantities under the real sample matrix condition. Each dilution point was analyzed in triplicate using PRM and the linearity of the generated curves was evaluated by regression analysis. The LOD and LOQ were determined based on the derived linear curves (Materials and Methods). The top four transitions from each peptide were used to calculate the response ratios with regards to reduce the influence of ion selection and improve the assay robustness.

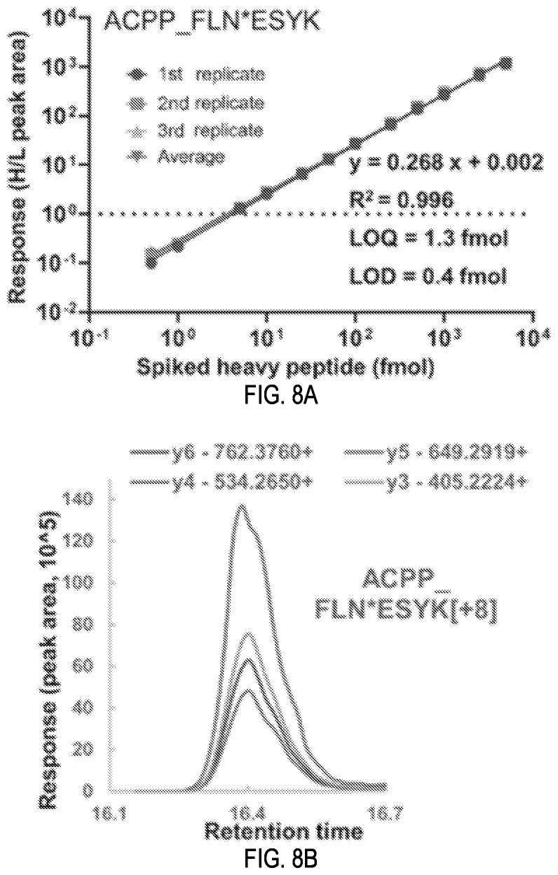

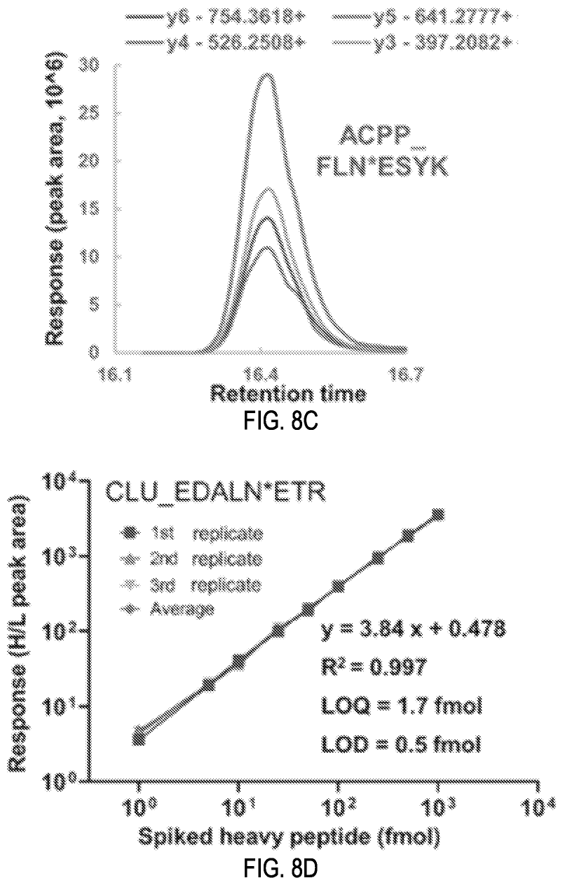

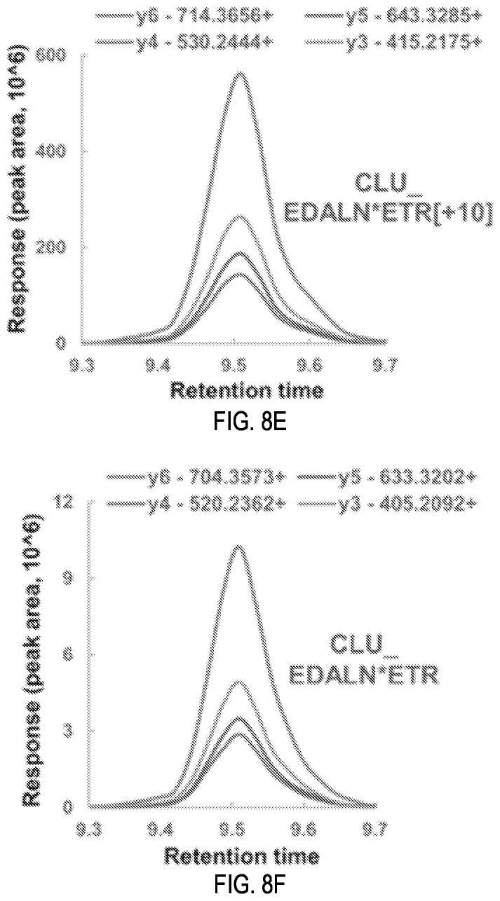

The generated 12-point linear curves for the heavy isotope-labeled glycopeptide of ACPP (FLN*ESYK[+8]) (SEQ ID NO:1) indicated that the working range of the PRM assay covered about four orders of magnitude from 1.3 fmol to 5,000 fmol, with the coefficient of determination value (R²) of 0.996 (FIG. 8A). The LOD and LOQ of the PRM assay for glycopeptide FLN*ESYK (SEQ ID NO:1) from ACPP were determined to be 0.4 fmol and 1.3 fmol, respectively. In addition, the extracted ion chromatography for PRM transitions of this glycopeptide and its heavy isotope-labeled counterpart demonstrated a consistency among the transitions since the transitions had similar peak shapes and retention time indicating no interference with contaminants and thus reliable quantification was achieved (FIG. 8B-8C). For the heavy isotope-labeled glycopeptide EDALN*ETR[+10] (SEQ ID NO:3) from CLU, as the peptide concentration increased, the linear correlation between the concentration and MS signal response became weaker; thus, data points that were outside the linear range were therefore removed. Consequently, a 9-point linear calibration curve was constructed with R² of 0.997; the effective working range ranged from 1.7 fmol to 1000 fmol, which covered three orders of magnitude (FIG. 8D). Reliable quantification of the glycopeptides was observed with consistency among different transitions (FIG. 8E-8F). Overall, LOD and LOQ of glycopeptide EDALN*ETR (SEQ ID NO:3) from CLU were 0.5 and 1.7 fmol, respectively.

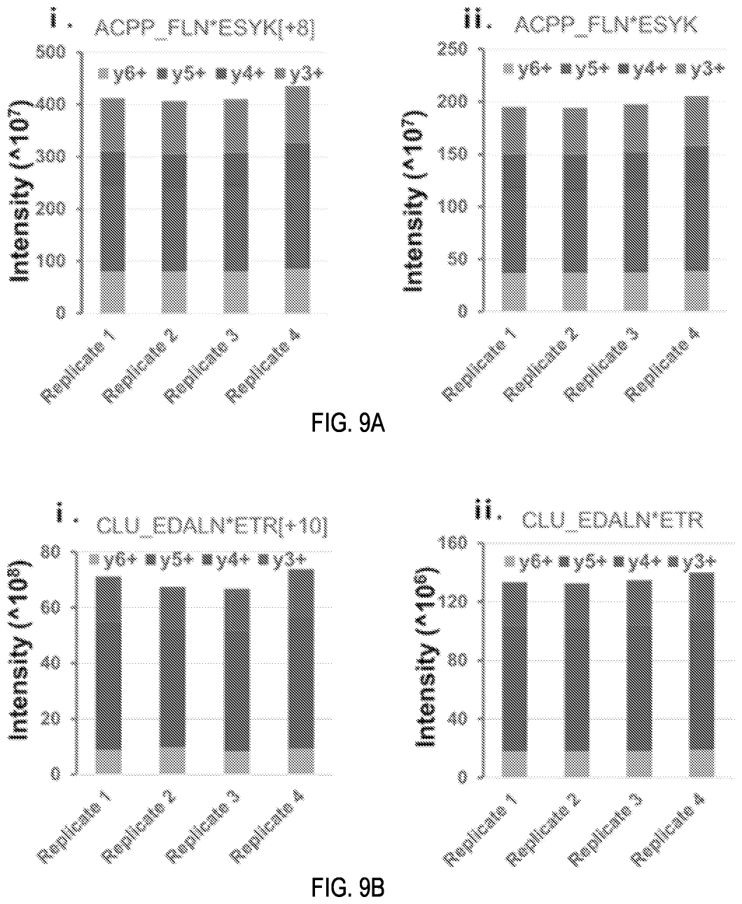

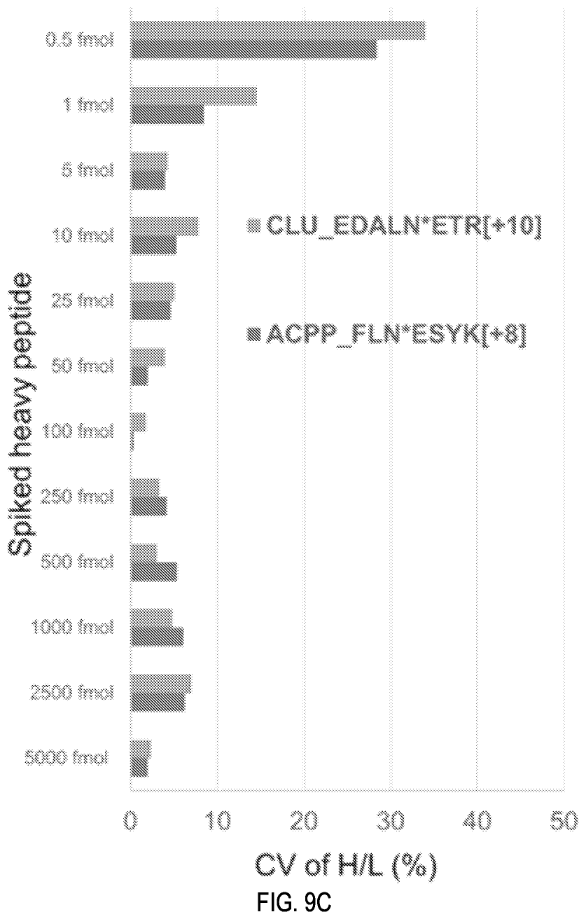

Repeatability of PRM quantification is crucial in candidate biomarker assessment, especially when a large cohort of samples is analyzed since it will take a much longer period of time for data collection. To examine the intra-day (with-in day) variability of the PRM assays, four repeated PRM analyses were conducted using the same test sample on the same day (the test sample were prepared by the addition of 0.1 pmol heavy isotope-labeled peptides per injection into the urine-derived glycopeptide background). The obtained peak area for each transition as well as for each peptide across the four repeated analyses was compared (FIG. 9A-9B). The coefficient of variations (CVs) for the peptides and transitions were less than 6% (Supplemental Table S2 (not shown)), indicated the consistency of PRM measurement (FIG. 9A-9B). To assess the inter-day variance of the PRM assays, the present inventors further utilized the data collected for the response curves to conduct a validation since (1) the replicates of each concentration were collected across 5 days, which was suitable for inter-day variation evaluation; (2) the amount of the heavy isotope-labeled peptides spanning from 0.5 fmol to 5,000 fmol, covered the entire working range of the established PRM assays. As shown in FIG. 9C, the CVs for the peptide response are less than 10% (on average 4.4%) with at least 5 fmol peptides. On the other hand, for the two data points with the lowest concentration (1 fmol or 0.5 fmol spike-in heavy peptides), relatively larger CVs were obtained. Notably, when a medium abundance (around 100 fmol) of heavy peptides were analyzed in the urinary glycopeptide sample, the PRM assays for both glycopeptides (FLN*ESYK (SEQ ID NO:1) and EDALN*ETR (SEQ ID NO:3)) yielded the smallest CVs, which were less than 2% (FIG. 9C). Therefore, 150 fmol heavy isotope-labeled peptides were spiked into the individual urine-derived glycopeptide sample in the present inventors' following experiment. In summary, PRM assays of both urinary glycopeptides from ACPP and CLU were successfully established with good repeatability and sufficient working range covering three to four orders of magnitude.

Implementation of the developed PRM assays to individual urine specimens. To facilitate the translation of candidate markers that the present inventors previously discovered for aggressive PCa using a DIA approach into a clinical setting in the future, the present inventors applied the PRM assays to the quantitative analysis of glycopeptides enriched from PCa urine specimens in order to evaluate their discrimination power in differentiating aggressive and non-aggressive PCa via such targeted glycoproteomic approach using PRM. First, glycopeptides isolated from individual urine samples were analyzed by PRM together with heavy isotope-labeled internal standard peptides. Upon examining the extracted ion chromatography of the transitions from the endogenous glycopeptides to ensure accuracy in peak picking, the present inventors found that the two targeted glycopeptides were quantified in all of the 142 urine samples with diverse intensities (Supplemental FIG. S1A (not shown) and S1C). For example, the intensity of FLN*ESYK (SEQ ID NO:1) from sample S124 was 1440 times higher than sample S114 (Supplemental FIG. S1A (not shown)). The diversity among the clinical samples were plausible since each sample had distinct abundances of candidate proteins. On the other hand, the MS intensities of heavy isotope-labeled peptides were less diverse and more evenly distributed among the samples since the same level of peptides were spiked into each sample (Supplemental FIG. S2A and S2C (not shown)). The variation of heavy isotope-labeled peptides was expected since each sample had distinct abundances of proteins and glycopeptides, which contributed to the differences in signal suppression effect of the present inventors' heavy isotope-labeled peptides. Therefore, the addition of heavy isotope-labeled internal standard peptides is essential to achieve accurate quantification result by utilizing the ratio between endogenous glycopeptides and their heavy isotope-labeled counterparts for relative quantification of the present inventors' endogenous glycopeptides. Although, the MS intensities of the PRM transitions seemed distinct among samples, however, the proportion of intensity (i.e., the proportion of each transition to the sum of the four transitions in each sample) of each transition was consistent across the 142 samples (Supplemental FIG. S1B, S1D, S2B and S2D (not shown)) implying the stable fragment pattern and the correct determination of the targeted glycopeptides. Hence, the responses of the endogenous glycopeptides relative to heavy were exported from Skyline and used for further statistical analysis.

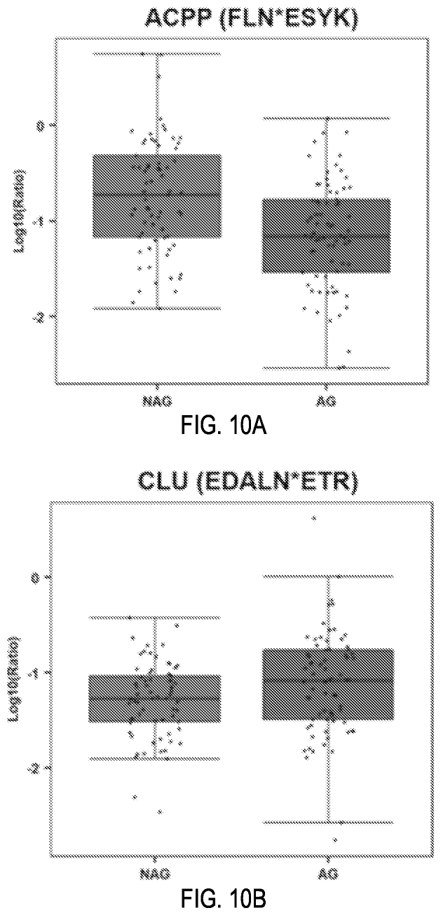

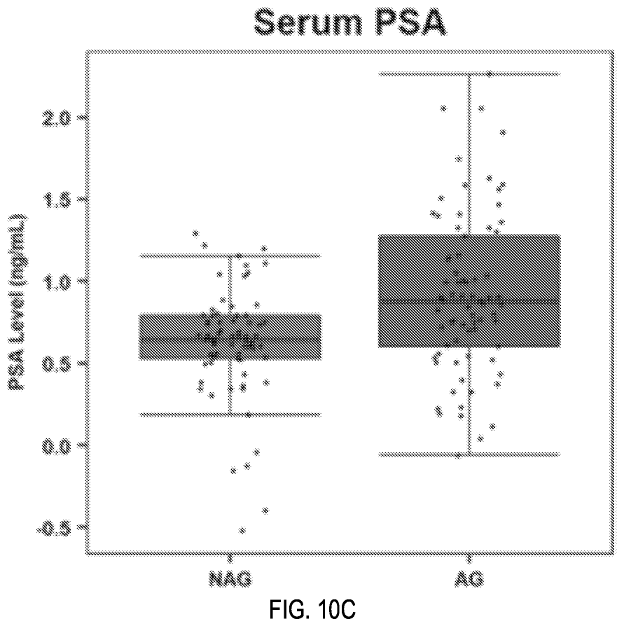

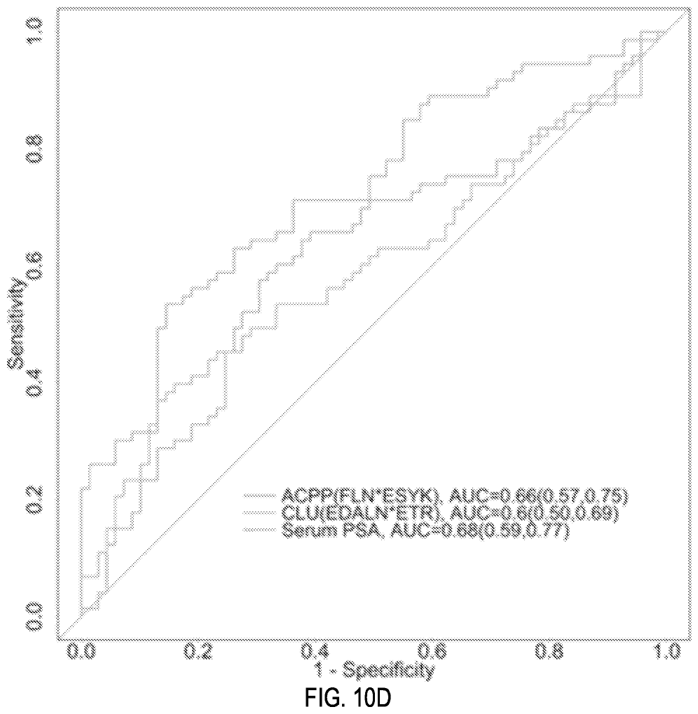

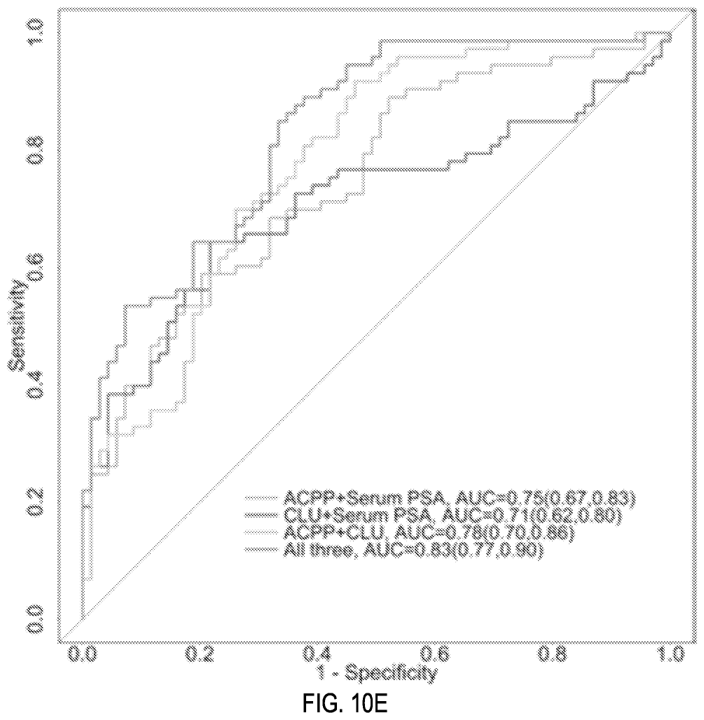

Evaluation of the performance of the PRM assays in detecting Aggressive PCa. To examine the performance of the glycopeptides from ACPP and CLU in distinguishing groups of samples with different aggressiveness of PCa, PRM quantification results (Supplemental Table S3 (not shown)) of the targeted glycopeptides were statistically analyzed. The expression profile of the glycopeptide from ACPP was significantly decreased in aggressive PCa relative to non-aggressive PCa, with 2.68-fold differences (p-value<0.01; FIG. 10A and Supplemental Table S4 (not shown)). By contrast, the glycopeptide from CLU showed higher expression level in the aggressive PCa group with a fold change of 1.54 (p-value<0.05; FIG. 10B and Supplemental Table S4 (not shown)). Using serum PSA as a reference, the present inventors observed that the median of total serum PSA levels in aggressive PCa group was 1.73 times higher than the non-aggressive group (FIG. 10C and Supplemental Table S4 (not shown)).

The present inventors further evaluated the discriminatory power of the PRM assays of glycopeptides from ACPP and CLU as well as serum PSA test as an individual marker signature in separating the two PCa subgroups by ROC analysis. As shown in FIG. 10D, ACPP has an AUC of 0.66 (95% CI: 0.57 to 0.75), while CLU has an AUC of 0.60 (95% CI: 0.50 to 0.69) (Supplemental Table S5 (not shown)) suggesting the glycopeptide from ACCP has better separation capability. However, when the two glycopeptides were combined together to form a two-signature panel, the predictive power towards aggressive PCa increased with an AUC of 0.78 (95% CI: 0.70 to 0.86; specificity of 53.6% at 91.8% sensitivity) (FIG. 10E and Supplemental Table S5 (not shown)). Serum PSA had a moderate performance as an individual signature (AUC of 0.68, 95% CI: 0.59 to 0.77, FIG. 10D); as the present inventors combined ACPP, CLU and serum PSA to create a three-signature panel, both urinary ACPP and CLU could supplement serum PSA test to improve the overall predictive power (FIG. 10E and Supplemental Table S5 (not shown)) achieving an AUC of 0.83 (95% CI: 0.77 to 0.90; specificity of 66.7% at 84.9% sensitivity). By further evaluating the PRM assays using urine specimens from an independent cohort (13 non-aggressive and 40 aggressive PCa patients), the present inventors observed similar outcome for the glycopeptide from ACCP (AUC=0.71, 95% CI: 0.54 to 0.87) and the two-signature panel composed of ACPP and serum PSA (AUC=0.81, 95% CI: 0.68 to 0.93). Detailed PRM quantification and results of the second cohort are in Supplemental Tables S6-7 (not shown). Compared with the present inventors' previous study using DIA, the present inventors observed similar outcome using the current targeted PRM methods. The two glycopeptides from urinary ACPP and CLU demonstrated consistent performance and capability in differentiating different PCa subgroups. Therefore, the results of this study elucidated that the PRM assays were successfully developed for urinary glycopeptides and were applicable to the quantitative analysis of targeted peptides from real clinical specimens as well as supplementing serum PSA test to gain improved discrimination power.

### Discussion

To avoid the possibility of overtreating indolent PCa as well as providing appropriate therapy to high-risk PCa patients, it is crucial to develop easy-adapted techniques with high precision to make candidate biomarkers clinically available for detecting aggressive PCa to aid patient risk stratification and therapy selection. The present inventors previously discovered two urinary glycopeptides, FLN*ESYK (SEQ ID NO:1) from ACPP and EDALN*ETR (SEQ ID NO:3) from CLU, as candidate markers for detecting aggressive PCa via DIA-approach¹⁸. To facilitate the translation of MS-discovered candidates to clinical lab as well as to further clarify their clinical utility, robust and accurate PRM assays that can be easily adopted in multi-labs were developed in the current study (FIG. 7).

Non-invasive liquid biopsy based on urine is a tool for detecting cancer and monitoring cancer progression, which has received great attention³³. However, high composition complexity of urine and the possibility of contamination can cause ionic suppression of the targeted peptides when analyzed under complex sample background. Therefore, it poses a great challenge for LC-MS/MS based quantification and hinder the quantitative accuracy³⁴. To circumvent these issues, reversed calibration curves were generated using a serial dilution of heavy isotope-labeled standard peptides (0.5 fmol to 5,000 fmol) spiking into the sample matrix background to establish the linear relationship between MS signals of targeted glycopeptides and their quantities under the real sample matrix condition; thus, ensuring the detected MS signals falling into the linear detection range and reflect true abundance of the targeted peptides. Each dilution point was analyzed in triplicate using PRM for the glycopeptides of ACPP and CLU (FIG. 8). The working range of the PRM assay for urinary ACPP covered from 1.3 fmol (LOQ) to 5000 fmol with LOD of 0.4 fmol, whereas the working range was 1.7 fmol (LOQ) to 1000 fmol for urinary CLU with 0.5 fmol. By assessing intra-day and inter-day variabilities of the established PRM assays, CVs <6% and <10% were achieved, respectively, indicating the PRM assays had good repeatability (FIG. 9).

The developed PRM assays were applied to 142 PCa urine specimens for evaluating their performances in detecting aggressive PCa (FIG. 10). Compared to the performance of individual candidates, a two-signature panel consisted of urinary CLU and urinary ACPP as well as the three-signature panel with both glycoproteins and serum PSA achieved better performance in differentiating aggressive PCa and non-aggressive PCa with AUCs of 0.78 and 0.83, respectively (FIG. 10E). Since serum PSA test along can only provide an AUC of 0.68 indicating the established PRM assays for urinary glycoproteins can be effective supplements to serum PSA test in PCa stratification. Similar outcome was achieved using the current targeted PRM methods compared to the present inventors' previous study using DIA approach suggesting consistent performance of urinary ACPP and CLU in detecting aggressive PCa. In summary, the present inventors developed PRM assays for urinary glycopeptides from CLU and ACPP showing high precision and providing reliable results in detecting aggressive PCa as well as in supplementing serum PSA test to further improve discrimination power towards the diagnosis of aggressive PCa. The developed PRM assays can be applied to large sample cohort or multi-center study for further validation.

The expression profile of glycopeptides may be attributable to either the expression of their corresponding proteins or the glycan occupancy of the glycosylation sites. Measurement on the total protein level can be an alternative strategy if the expression profiles of the protein and the glycopeptide shows similar trend. However, it will not work if the change in glycopeptide abundance is due to the increase in glycosylation occupancy. However, PRM assays specifically developed for glycopeptides can work in both situations.

In this study, the present inventors intended to measure the abundance of targeted glycopeptides enriched from the same volume of urine; thus, the abundance of targeted glycopeptides from the same volume of urine (50 μL, one-tenth of 500 μL) were compared across the whole sample cohort without further normalization. The rationale of using the same volume of urine for prostate protein analysis assumes that the abundance of proteins secreted from prostate to urine is independent of rest of protein contents from urine due to fact that urine collects prostatic components in urethra, while total urine protein content can come from kidney and bladder.

### References

(1) Siegel, R. L.; Miller, K. D.; Jemal, A. Cancer Statistics, 2020. CAA Cancer J Clin 2020, 70 (1), 7-30. https://doi.org/10.3322/caac.21590.
(2) Nowinski, S.; Santaolalla, A.; O'Leary, B.; Loda, M.; Mirchandani, A.; Emberton, M.; Van Hemelrijck, M.; Grigoriadis, A. Systematic Identification of Functionally Relevant Risk Alleles to Stratify Aggressive versus Indolent Prostate Cancer. Oncotarget 2018, 9 (16), 12812-12824. https://doi.org/10.18632/oncotarget.24400.
(3) Murphy, K.; Murphy, B. T.; Boyce, S.; Flynn, L.; Gilgunn, S.; O3 Rourke, C. J.; Rooney, C.; Stöckmann, H.; Walsh, A. L.; Finn, S.; O'Kennedy, R. J.; O'Leary, J.; Pennington, S. R.; Perry, A. S.; Rudd, P. M.; Saldova, R.; Sheils, O.; Shields, D. C.; Watson, R. W. Integrating Biomarkers across Omic Platforms: An Approach to Improve Stratification of Patients with Indolent and Aggressive Prostate Cancer. Mol Oncol 2018, 12 (9), 1513-1525. https://doi.org/10.1002/1878-0261.12348.
(4) Buyyounouski, M. K.; Choyke, P. L.; McKenney, J. K.; Sartor, O.; Sandler, H. M.; Amin, M. B.; Kattan, M. W.; Lin, D. W. Prostate Cancer—Major Changes in the American Joint Committee on Cancer Eighth Edition Cancer Staging Manual: Prostate Cancer-Major 8th Edition Changes. CA: A Cancer Journal for Clinicians 2017, 67 (3), 245-253. https://doi.org/10.3322/caac.21391.
(5) Eskra, J. N.; Rabizadeh, D.; Pavlovich, C. P.; Catalona, W. J.; Luo, J. Approaches to Urinary Detection of Prostate Cancer. Prostate Cancer Prostatic Dis 2019, 22 (3), 362-381. https://doi.org/10.1038/s41391-019-0127-4.
(6) Rzhevskiy, A. S.; Razavi Bazaz, S.; Ding, L.; Kapitannikova, A.; Sayyadi, N.; Campbell, D.; Walsh, B.; Gillatt, D.; Ebrahimi Warkiani, M.; Zvyagin, A. V. Rapid and Label-Free Isolation of Tumour Cells from the Urine of Patients with Localised Prostate Cancer Using Inertial Microfluidics. Cancers 2019, 12 (1), 81. https://doi.org/10.3390/cancers12010081.
(7) Donovan, M. J.; Noerholm, M.; Bentink, S.; Belzer, S.; Skog, J.; O'Neill, V.; Cochran, J. S.; Brown, G. A. A Molecular Signature of PCA3 and ERG Exosomal RNA from Non-DRE Urine Is Predictive of Initial Prostate Biopsy Result. Prostate Cancer Prostatic Dis 2015, 18 (4), 370-375. https://doi.org/10.1038/pcan.2015.40.
(8) Robert, G.; Jannink, S.; Smit, F.; Aalders, T.; Hessels, D.; Cremers, R.; Mulders, P. F.; Schalken, J. A. Rational Basis for the Combination of PCA3 and TMPRSS2:ERG Gene Fusion for Prostate Cancer Diagnosis. Prostate 2013, 73 (2), 113-120. https://doi.org/10.1002/pros.22546.
(9) Wei, J. T.; Feng, Z.; Partin, A. W; Brown, E.; Thompson, I.; Sokoll, L.; Chan, D. W.; Lotan, Y.; Kibel, A. S.; Busby, J. E.; Bidair, M.; Lin, D. W; Taneja, S. S.; Viterbo, R.; Joon, A. Y.; Dahlgren, J.; Kagan, J.; Srivastava, S.; Sanda, M. G. Can Urinary PCA3 Supplement PSA in the Early Detection of Prostate Cancer? JCO 2014, 32 (36), 4066-4072. https://doi.org/10.1200/JC0.2013.52.8505.
(10) Leyten, G. H. J. M.; Hessels, D.; Jannink, S. A.; Smit, F. P.; de Jong, H.; Cornel, E. B.; de Reijke, T. M.; Vergunst, H.; Kil, P.; Knipscheer, B. C.; van Oort, I. M.; Mulders, P. F. A.; Hulsbergen-van de Kaa, C. A.; Schalken, J. A. Prospective Multicentre Evaluation of PCA3 and TMPRSS2-ERG Gene Fusions as Diagnostic and Prognostic Urinary Biomarkers for Prostate Cancer. European Urology 2014, 65 (3), 534-542. https://doi.org/10.1016/j.eururo.2012.11.014.
(11) Rittenhouse, H.; Blase, A.; Shamel, B.; Schalken, J.; Groskopf, J. The Long and Winding Road to FDA Approval of a Novel Prostate Cancer Test: Our Story. Clinical Chemistry 2013, 59 (1), 32-34. https://doi.org/10.1373/clinchem.2012.198739.
(12) Tomlins, S. A.; Day, J. R.; Lonigro, R. J.; Hovelson, D. H.; Siddiqui, J.; Kunju, L. P.; Dunn, R. L.; Meyer, S.; Hodge, P.; Groskopf, J.; Wei, J. T.; Chinnaiyan, A. M. Urine TMPRSS2:ERG Plus PCA3 for Individualized Prostate Cancer Risk Assessment. European Urology 2016, 70 (1), 45-53. https://doi.org/10.1016/j.eururo.2015.04.039.
(13) McKiernan, J.; Donovan, M. J.; O'Neill, V.; Bentink, S.; Noerholm, M.; Belzer, S.; Skog, J.; Kattan, M. W.; Partin, A.; Andriole, G.; Brown, G.; Wei, J. T.; Thompson, I. M.; Carroll, P. A Novel Urine Exosome Gene Expression Assay to Predict High-Grade Prostate Cancer at Initial Biopsy. JAMA Oncol 2016, 2 (7), 882. https://doi.org/10.1001/jamaonco1.2016.0097.
(14) Leyten, G. H. J. M.; Hessels, D.; Smit, F. P.; Jannink, S. A.; de Jong, H.; Melchers, W. J. G.; Cornel, E. B.; de Reijke, T. M.; Vergunst, H.; Kil, P.; Knipscheer, B. C.; Hulsbergen-van de Kaa, C. A.; Mulders, P. F. A.; van Oort, I. M.; Schalken, J. A. Identification of a Candidate Gene Panel for the Early Diagnosis of Prostate Cancer. Clin Cancer Res 2015, 21 (13), 3061-3070. https://doi.org/10.1158/1078-0432.CCR-14-3334.
(15) Kim, Y.; Jeon, J.; Mejia, S.; Yao, C. Q.; Ignatchenko, V.; Nyalwidhe, J. O.; Gramolini, A. O.; Lance, R. S.; Troyer, D. A.; Drake, R. R.; Boutros, P. C.; Semmes, O. J.; Kislinger, T. Targeted Proteomics Identifies Liquid-Biopsy Signatures for Extracapsular Prostate Cancer. Nat Commun 2016, 7 (1), 11906. https://doi.org/10.1038/ncomms11906.
(16) Sequeiros, T.; Rigau, M.; Chiva, C.; Montes, M.; Garcia-Grau, I.; Garcia, M.; Diaz, S.; Celma, A.; Bijnsdorp, I.; Campos, A.; Di Mauro, P.; Borros, S.; Reventos, J.; Doll, A.; Paciucci, R.; Pegtel, M.; de Torres, I.; Sabido, E.; Morote, J.; Olivan, M. Targeted Proteomics in Urinary Extracellular Vesicles Identifies Biomarkers for Diagnosis and Prognosis of Prostate Cancer. Oncotarget 2017, 8 (3), 4960-4976. https://doi.org/10.18632/oncotarget.13634.
(17) Hendriks, R. J.; van Oort, I. M.; Schalken, J. A. Blood-Based and Urinary Prostate Cancer Biomarkers: A Review and Comparison of Novel Biomarkers for Detection and Treatment Decisions. Prostate Cancer Prostatic Dis 2017, 20 (1), 12-19. https://doi.org/10.1038/pcan.2016.59.
(18) Dong, M.; Lih, T. M.; Chen, S.-Y.; Cho, K.-C.; Eguez, R. V.; Hob, N.; Zhou, Y; Yang, W.; Mangold, L.; Chan, D. W.; Zhang, Z.; Sokoll, L. J.; Partin, A.; Zhang, H. Urinary Glycoproteins Associated with Aggressive Prostate Cancer. Theranostics 2020, 10 (26), 11892-11907. https://doi.org/10.7150/thno.47066.
(19) Gutman, A. B.; Gutman, E. B. AN “ACID” PHOSPHATASE OCCURRING IN THE SERUM OF PATIENTS WITH METASTASIZING CARCINOMA OF THE PROSTATE GLAND. J. Clin. Invest. 1938, 17 (4), 473-478. https://doi.org/10.1172/JCI100974.
(20) Stamey, T. A.; Yang, N.; Hay, A. R.; McNeal, J. E.; Freiha, F. S.; Redwine, E. Prostate-Specific Antigen as a Serum Marker for Adenocarcinoma of the Prostate. N Engl J Med 1987, 317 (15), 909-916. https://doi.org/10.1056/NEJM198710083171501.
(21) Uhlen, M.; Fagerberg, L.; Hallstrom, B. M.; Lindskog, C.; Oksvold, P.; Mardinoglu, A.; Sivertsson, A.; Kampf, C.; Sjostedt, E.; Asplund, A.; Olsson, I.; Edlund, K.; Lundberg, E.; Navani, S.; Szigyarto, C. A.-K.; Odeberg, J.; Djureinovic, D.; Takanen, J. O.; Hober, S.; Alm, T.; Edqvist, P.-H.; Berling, H.; Tegel, H.; Mulder, J.; Rockberg, J.; Nilsson, P.; Schwenk, J. M.; Hamsten, M.; von Feilitzen, K.; Forsberg, M.; Persson, L.; Johansson, F.; Zwahlen, M.; von Heijne, G.; Nielsen, J.; Ponten, F. Tissue-Based Map of the Human Proteome. Science 2015, 347 (6220), 1260419-1260419. https://doi.org/10.1126/science.1260419.
(22) Rizzi, F.; Bettuzzi, S. The Clusterin Paradigm in Prostate and Breast Carcinogenesis. Endocrine-Related Cancer 2010, 17 (1), R1-R17. https://doi.org/10.1677/ERC-09-0140.
(23) Koltai, T. Clusterin: A Key Player in Cancer Chemoresistance and Its Inhibition. OTT 2014, 447. https://doi.org/10.2147/OTT.S58622.
(24) Bonacini, M.; Negri, A.; Davalli, P.; Naponelli, V.; Ramazzina, I.; Lenzi, C.; Bettuzzi, S.; Rizzi, F. Clusterin Silencing in Prostate Cancer Induces Matrix Metalloproteinases by an NF-κ B-Dependent Mechanism. Journal of Oncology 2019, 2019, 1-12. https://doi.org/10.1155/2019/4081624.
(25) Addona, T. A.; Abbatiello, S. E.; Schilling, B.; Skates, S. J.; Mani, D. R.; Bunk, D. M.; Spiegelman, C. H.; Zimmerman, L. J.; Ham, A.-J. L.; Keshishian, H.; Hall, S. C.; Allen, S.; Blackman, R. K.; Borchers, C. H.; Buck, C.; Cardasis, H. L.; Cusack, M. P.; Dodder, N. G.; Gibson, B. W.; Held, J. M.; Hiltke, T.; Jackson, A.; Johansen, E. B.; Kinsinger, C. R.; Li, J.; Mesri, M.; Neubert, T. A.; Niles, R. K.; Pulsipher, T. C.; Ransohoff, D.; Rodriguez, H.; Rudnick, P. A.; Smith, D.; Tabb, D. L.; Tegeler, T. J.; Variyath, A. M.; Vega-Montoto, L. J.; Wahlander, A.; Waldemarson, S.; Wang, M.; Whiteaker, J. R.; Zhao, L.; Anderson, N. L.; Fisher, S. J.; Liebler, D. C.; Paulovich, A. G.; Regnier, F. E.; Tempst, P.; Carr, S. A. Multi-Site Assessment of the Precision and Reproducibility of Multiple Reaction Monitoring-Based Measurements of Proteins in Plasma. Nat Biotechnol 2009, 27 (7), 633-641. https://doi.org/10.1038/nbt.1546.
(26) Prakash, A.; Rezai, T.; Krastins, B.; Sarracino, D.; Athanas, M.; Russo, P.; Zhang, H.; Tian, Y.; Li, Y.; Kulasingam, V.; Drabovich, A.; Smith, C. R.; Batruch, I.; Oran, P. E.; Fredolini, C.; Luchini, A.; Liotta, L.; Petricoin, E.; Diamandis, E. P.; Chan, D. W.; Nelson, R.; Lopez, M. F. Interlaboratory Reproducibility of Selective Reaction Monitoring Assays Using Multiple Upfront Analyte Enrichment Strategies. J. Proteome Res. 2012, 11 (8), 3986-3995. https://doi.org/10.1021/pr300014s.
(27) Shrivastava, A.; Gupta, V. Methods for the Determination of Limit of Detection and Limit of Quantitation of the Analytical Methods. Chron Young Sci 2011, 2 (1), 21. https://doi.org/10.4103/2229-5186.79345.
(28) Kuhn, M. Building Predictive Models in R Using the Caret Package. J. Stat. Soft. 2008, 28 (5). https://doi.org/10.18637/jss.v028.i05.
(29) Robin, X.; Turck, N.; Hainard, A.; Tiberti, N.; Lisacek, F.; Sanchez, J.-C.; Muller, M. PROC: An Open-Source Package for R and S+to Analyze and Compare ROC Curves. BMC Bioinformatics 2011, 12 (1), 77. https://doi.org/10.1186/1471-2105-12-77.
(30) Clark, D. J.; Hu, Y; Schnaubelt, M.; Fu, Y.; Ponce, S.; Chen, S.-Y; Zhou, Y.; Shah, P.; Zhang, H. Simple Tip-Based Sample Processing Method for Urinary Proteomic Analysis. Analytical Chemistry 2019, 91 (9), 5517-5522. https://doi.org/10.1021/acs.analchem.8b05234.
(31) Chen, S.-Y.; Dong, M.; Yang, G.; Zhou, Y; Clark, D. J.; Lih, T. M.; Schnaubelt, M.; Liu, Z.; Zhang, H. Glycans, Glycosite, and Intact Glycopeptide Analysis of N-Linked Glycoproteins Using Liquid Handling Systems. Anal. Chem. 2020, 92 (2), 1680-1686. https://doi.org/10.1021/acs.analchem.9b03761.
(32) Whiteaker, J. R.; Halusa, G. N.; Hoofnagle, A. N.; Sharma, V.; MacLean, B.; Yan, P.; Wrobel, J. A.; Kennedy, J.; Mani, D. R.; Zimmerman, L. J.; Meyer, M. R.; Mesri, M.; Boja, E.; Carr, S. A.; Chan, D. W.; Chen, X.; Chen, J.; Davies, S. R.; Ellis, M. J. C.; Fenyö, D.; Hiltke, T.; Ketchum, K. A.; Kinsinger, C.; Kuhn, E.; Liebler, D. C.; Liu, T.; Loss, M.; MacCoss, M. J.; Qian, W.-J.; Rivers, R.; Rodland, K. D.; Ruggles, K. V.; Scott, M. G.; Smith, R. D.; Thomas, S.; Townsend, R. R.; Whiteley, G.; Wu, C.; Zhang, H.; Zhang, Z.; Rodriguez, H.; Paulovich, A. G. Using the CPTAC Assay Portal to Identify and Implement Highly Characterized Targeted Proteomics Assays. In Quantitative Proteomics by Mass Spectrometry; Sechi, S., Ed.; Springer New York: New York, NY, 2016; Vol. 1410, pp 223-236. https://doi.org/10.1007/978-1-4939-3524-6_13.
(33) Harlan, R.; Zhang, H. Targeted Proteomics: A Bridge between Discovery and Validation. Expert Review of Proteomics 2014, 11 (6), 657-661. https://doi.org/10.1586/14789450.2014.976558.
(34) Hoofnagle, A. N.; Whiteaker, J. R.; Carr, S. A.; Kuhn, E.; Liu, T.; Massoni, S. A.; Thomas, S. N.; Townsend, R. R.; Zimmerman, L. J.; Boja, E.; Chen, J.; Crimmins, D. L.; Davies, S. R.; Gao, Y; Hiltke, T. R.; Ketchum, K. A.; Kinsinger, C. R.; Mesri, M.; Meyer, M. R.; Qian, W-J.; Schoenherr, R. M.; Scott, M. G.; Shi, T.; Whiteley, G. R.; Wrobel, J. A.; Wu, C.; Ackermann, B. L.; Aebersold, R.; Barnidge, D. R.; Bunk, D. M.; Clarke, N.; Fishman, J. B.; Grant, R. P.; Kusebauch, U.; Kushnir, M. M.; Lowenthal, M. S.; Moritz, R. L.; Neubert, H.; Patterson, S. D.; Rockwood, A. L.; Rogers, J.; Singh, R. J.; Van Eyk, J. E.; Wong, S. H.; Zhang, S.; Chan, D. W; Chen, X.; Ellis, M. J.; Liebler, D. C.; Rodland, K. D.; Rodriguez, H.; Smith, R. D.; Zhang, Z.; Zhang, H.; Paulovich, A. G. Recommendations for the Generation, Quantification, Storage, and Handling of Peptides Used for Mass Spectrometry-Based Assays. Clinical Chemistry 2016, 62 (1), 48-69. https://doi.org/10.1373/clinchem.2015.250563.
(35) Desiere, F.; Deutsch, E. W.; King, N. L.; Nesvizhskii, A. I.; Mallick, P.; Eng, J.; Chen, S.; Eddes, J.; Loevenich, S. N.; Aebersold, R. The PeptideAtlas Project. Nucleic Acids Res 2006, 34 (Database issue), D655-658. https://doi.org/10.1093/nar/gkj040.
(36) Clark D J, Hu Y, Schnaubelt M, Fu Y, Ponce S, Chen S-Y, Zhou Y, et al. Simple Tip-Based Sample Processing Method for Urinary Proteomic Analysis. Anal Chem 2019;91:5517-22.
(37) Chen S Y, Dong M, Yang G, Zhou Y, Clark D J, Lih T M, Schnaubelt M, et al. Glycans, Glycosite, and Intact Glycopeptide Analysis of N-Linked Glycoproteins Using Liquid Handling Systems. Anal Chem 2020;92:1680-6.
(38) Rappsilber J, Mann M, Ishihama Y. Protocol for micro-purification, enrichment, pre-fractionation and storage of peptides for proteomics using StageTips. Nat Protoc 2007;2:1896-906.

### Example 3: Development and Validation in vitro Diagnostic Multivariate Index Assays (IVDMIA) Using Data-Dependent Acquisition (DIA), Parallel Reaction Monitoring (PRM), and Computational Models to Combine a Panel of Biomarkers into a Single-Valued Numerical Index

Urine sample cohorts. A discovery cohort containing post-digital rectal examination (DRE) urine samples from 74 aggressive prostate cancer (AG PCa) patients (Gleason score≥8) and 68 non-aggressive (NAG) PCa patients (Gleason score=6) were analyzed using data-independent acquisition (DIA) mass spectrometry (MS). To further evaluate the predictive models, we used an independent validation cohort composed of 51 NAG PCa and 143 AG PCa, post-DRE urine samples. For the validation cohort, 38 NAG PCa, samples were upgraded to higher Gleason scores based on the Gleason scores from radical retropubic prostatectomy (RRP). All the urine samples were collected by the Department of Urology at Johns Hopkins University School of Medicine with approval from the Institutional Review Board of Johns Hopkins University under informed consent.

Urinary glycopeptide profiling using DIA-MS. For the quantitative analysis of glycopeptides in the discovery and validation sets, DIA raw data files were first searched against the prostate cancer specific glycopeptide spectral library for identification of glycopeptides followed by the quantification via Spectronaut Pulsar X. We performed normalization on glycopeptides to the total protein amount in individual urine sample. The glycopeptides were subjected to the downstream statistical analysis. Detailed experimental information can be found in our previous published work (PMID: 33204318).

Statistical analysis. For each candidate marker, its discriminatory power as an individual marker or in combination with other urinary glycopeptides and/or urine PSA through logistic regression was evaluated using receiver operating characteristic (ROC) analysis. The candidate marker data (missing values were median imputed) were log-transformed followed by z-score prior to ROC analysis. To ensure statistical stability of the results, we used bootstrap resampling (n=500) of the data. The mean ROC curves were depicted from the results of the aforementioned method and area under the curve (AUC) was computed for the mean ROC curve of a predictive model. The p-value for each glycopeptide in the discovery cohort was computed between AG and NAG groups using Mann-Whitney U test. The generated predictive models were further investigated using the independent validation cohort.

All the analyses were carried out in R (version 3.5). The predictive models were built using caret (version 6.0-85) and ROC curves were generated using pROC (version 1.13).

Performance of urinary marker panels from DIA data. The present inventors investigated four urinary glycopeptides corresponding to four glycoproteins showed significant difference between AG PCa and NAG PCa in addition to urine PSA in Table 11 since they showed good performance in detecting AG PCa based on ROC analysis.

TABLE 11
Assessment of the difference between AG and NAG
groups from discovery cohort
(p < 0.05 considered as significant).

| Individual candidate marker | p-value |
|---|---|
| ACPP (FLN*ESYK) (SEQ ID NO: 1) | 2.95E-06 |
| CLU (EDALN*ETR) (SEQ ID NO: 3) | 0.001923 |
| ORMI (QDQCIYN*TTYLNVQR) | 0.0008 |
| (SEQ ID NO: 5) | |
| CD97 (WCPQNSSCVN*ATACR) | 0.000141 |
| (SEQ ID NO: 21) | |
| Urine PSA | 5.72E-07 |

We observed that the urinary glycopeptides from CLU, ORM1, and CD97 showed moderate performance in separating AG and NAG PCa with AUCs ranged from 0.64 to 0.67 (Table 12). On the other hand, urinary glycopeptide of ACPP and urine PSA had better perfomiance compared to the above-mentioned glycopeptides. We obtained an AUC of 0.72 for urinary ACPP and an AUC of 0.73 for urine PSA. We also evaluated different combinations of the glycopeptides and/or urine PSA into multiple-marker panels. We observed improvement in the overall performance in detecting AG PCa using multiple-marker panels. As shown in Table 13 (for simplicity, only protein names are used in the table), the AUCs of multiple-marker panels are ranged from 0.77 to 0.85. Taken. together, the urinary glycopeptides and urine PSA, either individually or in combination with others, may be useful in AG PCa detection.

TABLE 12
Performance as individual candidate marker. 95% confidence interval (CI) are
displayed.

| | | | | | Specificity |
|---|---|---|---|---|---|
| Indivi- | | AUC | Specificity | Sensitivity | at 95% |
| dually | Panel | (95% CI) | (95% CI) | (95% CI) | sensitivity |
| | CLU | 0.64 | 30.9% | 91.9% | 27.94% |
| | (EDALN*ETR) (SEQ ID NO: 3) | (0.55, 0.73) | (21.9%, 39.9%) | (86.6%, 97.2%) | |
| | ORM1 | 0.66 | 41.2% | 89.2% | 33.82% |
| | (QDQCIYN*TTYLNVQR) | (0.57, 0.75) | (31.6%, 50.8%) | (83.1%, 95.2%) | |
| | (SEQ ID NO: 5) | | | | |
| | CD97 | 0.67 | 73.5% | 59.5% | 13.24% |
| | (WCPQNSSCVN*ATACR) | (0.58, 0.76) | (64.9%, 82.1%) | (49.9%, 69%) | |
| | (SEQ ID NO: 21) | | | | |
| | ACPP | 0.72 | 58.8% | 78.4% | 26.47% |
| | (FLN*ESYK) (SEQ ID NO: 1) | (0.64, 0.81) | (49.2%, 68.4%) | (70.3%, 86.4%) | |
| | Urine PSA | 0.73 | 67.6% | 73% | 26.47% |
| | | (0.65, 0.82) | (58.5%, 76.8%) | (64.3%, 81.6%) | |

TABLE 13
Performance of different combinations of the individual candidate markers from Table 12.

| | | | | | Specificity |
|---|---|---|---|---|---|
| | | AUC | Specificity | Sensitivity | at 95% |
| | Panel | (95% CI) | (95% CI) | (95% CI) | sensitivity |
| In | ACPP & CLU | 0.78 | 80.9% | 67.6% | 41.18% |
| combination | | (0.70, 0.86) | (73.2%, 88.6%) | (58.4%, 76.7%) | |
| | ACPP & ORM1 | 0.78 | 77.9% | 67.6% | 35.29% |
| | | (0.70, 0.85) | (69.9%, 86%) | (58.4%, 76.7%) | |
| | ACPP & CD97 | 0.77 | 63.2% | 82.4% | 26.47% |
| | | (0.69, 0.85) | (53.8%, 72.6%) | (75%, 89.9%) | |
| | ACPP, CLU& ORM1 | 0.82 | 72.1% | 81.1% | 44.12% |
| | | (0.75, 0.89) | (63.3%, 80.8%) | (73.4%, 88.7%) | |
| | ACPP, CLU& urine PSA | 0.79 | 60.3% | 87.8% | 39.71% |
| | | (0.71, 0.86) | (50.8%, 69.8%) | (81.5%, 94.2%) | |
| | ACPP, ORM1& urine PSA | 0.8 | 72.1% | 75.7% | 35.29% |
| | | (0.73, 0.87) | (63.3%, 80.8%) | (67.3%, 84%) | |
| | ACPP, CD97& urine PSA | 0.78 | 82.4% | 62.2% | 38.24% |
| | | (0.71, 0.86) | (74.9%, 89.8%) | (52.7%, 71.6%) | |
| | ACPP, CLU, ORM1& CD97 | 0.85 | 67.6% | 89.2% | 41.18% |
| | | (0.79, 0.91) | (58.5%, 76.8%) | (83.1%, 95.2%) | |
| | ACPP, CLU, ORM1& urine PSA | 0.83 | 73.5% | 81.1% | 48.53% |
| | | (0.76, 0.90) | (64.9%, 82.1%) | (73.4%, 88.7%) | |
| | | 0.85 | 75% | 82.4% | 48.53% |
| | ACPP, CLU, ORM1, CD97 & urine PSA | (0.79, 0.92) | (66.6%, 83.4%) | (75%, 89.9%) | |

Validation of the urinary marker panels using an independent validation cohort. The validation cohort contained a total of 194 urine samples, which was composed of 51 Gleason 6 (38 upgraded to higher Gleason groups), 57 Gleason 7 (3+4), 46 Gleason 7 (4+3), and 40 Gleason 8. Since our predictive models were built using urine samples of Gleason 6 and ≥Gleason 8, therefore, we first evaluated the performance of the panels using urine samples of Gleason 6 (excluding upgraded samples) and Gleason 8 from the validation cohort. ACPP and urine PSA as individual marker panel still showed good performance in distinguishing Gleason 6 from Gleason 8 with. AUCs of 0.74 and 0.78, respectively. Five multi-marker panels demonstrated moderate performance using the validation cohort (Table 14).

TABLE 14
Validation using Gleason 6 and 8 samples
from the independent cohort.

| Panel | AUC (95% CI) |
|---|---|
| ACPP | 0.74 (0.60, 0.88) |
| Urine PSA | 0.78 (0.65, 0.92) |
| ACPP & CD97 | 0.73 (0.58, 0.88) |
| ACPP, CLU & urine PSA | 0.71 (0.55, 0.88) |
| ACPP, ORM1 & urine PSA | 0.74 (0.60, 0.88) |
| ACPP, CD97 & urine PSA | 0.79 (0.66, 0.93) |
| ACPP, CLU, ORM1, CD97 & urine PSA | 0.72 (0.57, 0.88) |

PCa patients with either (3+4) or (4+) are all considered as in the intermediate risk. However, a PCa tumor classified as (3+4) contains more pattern 3 and a small portion of pattern 4, which may hamper the differentiation between Gleason 6 and (3+4) and above. Therefore, we examined the performance of the urinary marker panels in separating Gleason 6 from (3+4) and above as well as Gleason. 6 from (4+3) and above. We found urine PSA, a panel comprising ACPP, ORM1, and urine PSA, and a panel comprising ACPP, CLU and urine PSA showing promising results in detecting aggressive cancer with Gleason score (3+4) and above. Moreover, urine PSA had good performance (AUC=0.77) in distinguishing Gleason 6 and (4+3) and above along with three other urinary marker panels (Table 15). In addition, we investigated the urinary marker panels in predicting Gleason 6 upgrading and we found that CLU had the potential for such purpose with an AUC of 0.75 (95% CI: 0.57-0.94). Collectively, novel panels of candidate biomarkers for aggressive PCa were discovered showing promising results as further evaluated using an independent validation cohort.

TABLE 15
Validation using Gleason 6, 7, and 8
samples from the independent cohort.

| | |
|---|---|
| | AUC |
| Panel | (95% CI) |

Gleason 6 vs (3 + 4) and above

| | | |
|---|---|---|
| Urine PSA | 0.72 | (0.61, 0.84) |
| ACPP, ORM1 & urine PSA | 0.71 | (0.58, 0.83) |
| ACPP, CLU & urine PSA | 0.64 | (0.49, 0.79) |

Gleason 6 vs (4 + 3) and above

| | | |
|---|---|---|
| Urine PSA | 0.77 | (0.65, 0.88) |
| ACPP, CLU & urine PSA | 0.7 | (0.54, 0.85) |
| ACPP, ORM1 & urine PSA | 0.72 | (0.59, 0.85) |
| ACPP, CD97 & urine PSA | 0.71 | (0.57, 0.84) |

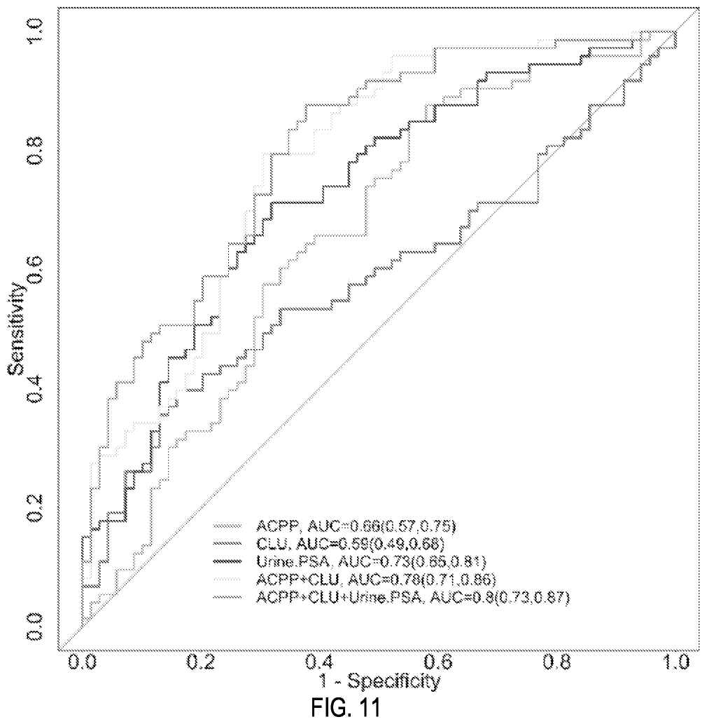

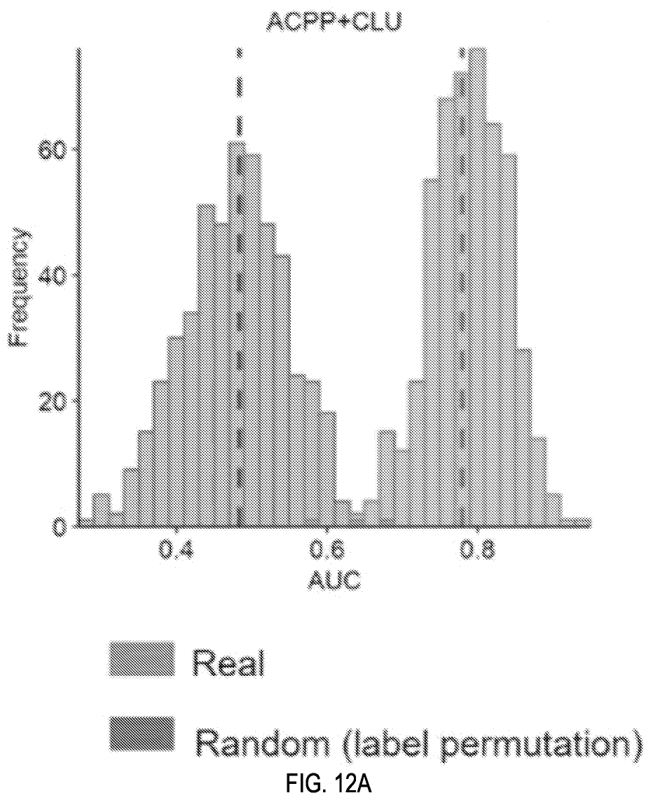

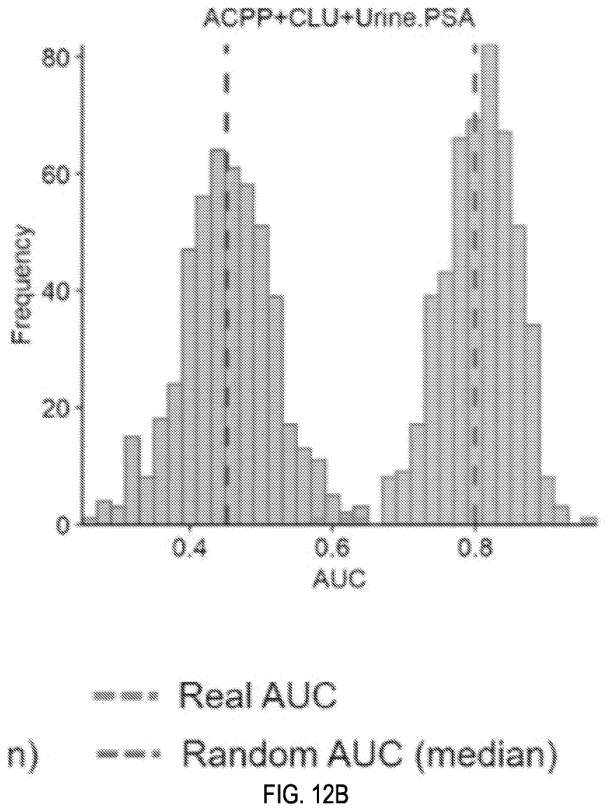

Performance of urinary marker panels from parallel reaction monitoring (PRM) assays. Two glycopeptides from urinary protein ACPP and CLU were discovered with promising results in detecting aggressive PCa based on quantitative DIA analysis. To evaluate the clinical utility of the candidate glycopeptides and facilitate the translation of the MS-based candidate biomarkers to routine clinical implementation in future, we developed easily extendable PRM quantitative assays for the aforementioned urinary glycopeptides (PMID: 34106707). We evaluated the performance of the two urinary glycopeptides along with urine PSA in the discovery cohort. As shown in FIG. 11, an improvement in differentiating AG and NAG PCa using a panel composed of ACPP and CLU (AUC=0.78) compared to individual candidate markers. An AUC of 0.8 was achieved when combining ACPP, CLU and urine PSA. To ensure the performance of the multi-marker panels, we generated and analyzed 500 random models (by label permutation of the original data) and computed the AUC for each random model (FIG. 12). The random models generated median AUCs of 0.48 and 0.47 for the panel of ACPP+CLU and the panel of ACPP+CLU+urine PSA, respectively, which were much lower and clearly separated from our real models.

To validate the performance of the PRM assays, we used the independent cohort. We found PRM assays of ACPP still maintained its abilities in differentiating Gleason 6 group from Gleason 8 group. Urine PSA also showed consistent performance in the group comparisons of (1) Gleason 6 vs Gleason 8, (2) Gleason 6 vs (3+4) and above, and (3) Gleason 6 vs (4+3) and above. The multi-marker panels showed moderate performance in the aforementioned group comparisons (Table 16).

In summary, the reported results elucidated that the PRM assays were successfully developed for urinary glycopeptides and were applicable to the quantitative analysis of targeted peptides from real clinical specimens as well as combining with urine PSA to gain improved discrimination power.

TABLE 16
Validation on PRM assays and urine PSA.

| | | Gleason 6 vs (3 + 4) | Gleason 6 vs (4 + 3) |
|---|---|---|---|
| | Gleason 6 vs 8 | and above | and above |
| | AUC | AUC | AUC |
| Panel | (95% CI) | (95% CI) | (95% CI) |
| ACPP (FLN*ESYK) | 0.71 | 0.66 | 0.68 |
| | (0.54, 0.87) | (0.50, 0.81) | (0.53, 0.84) |
| Urine PSA | 0.78 | 0.72 | 0.76 |
| | (0.65, 0.92) | (0.60, 0.84) | (0.65, 0.88) |
| ACPP + CLU | 0.65 | 0.62 | 0.68 |
| | (0.48, 0.82) | (0.47, 0.78) | (0.52, 0.84) |
| ACPP + CLU + | 0.65 | 0.62 | 0.68 |
| Urine PSA | (0.48, 0.82) | (0.48, 0.76) | (0.53, 0.84) |

TABLE 17
Sequences

| SEQ ID NO: | PROTEIN (PEPTIDE) (UNIPROT NO.) |
|---|---|
| 1 | ACPP (FLN*ESYK) |
| 2 | LOX (AEN*QTAPGEVPALSNLRPPSR) |
| 3 | CLU (EDALN*ETR) |
| 4 | SERPINA1 (YLGN*ATAIFFLPDEGK) |
| 5 | ORM1 (QDQCIYN*TTYLNVQR) |
| 6 | CD63 (CCGAAN*YTDWEK) |
| 7 | ATRN (ISN*SSDTVECECSENWK) |
| 8 | GP2 (QDLN*SSDVHSLQPQLDCGPR) |
| 9 | KLK11 (TATESFPHPGFN*NSLPNK) |
| 10 | PTPRN2 (VSANVQN*VTTEDVEK) |
| 11 | NPTN (AN*ATIEVK) |
| 12 | CPE (DLQGNPIAN*ATISVEGIDHDVTSAK) |
| 13 | RNASE2 (NQNTFLLTTFANVVNVCGNPN*MTCPSN*K) |
| 14 | DSC2 (NGIYN*ITVLASDQGGR) |
| 15 | LRG1 (LPPGLLAN*FTLLR) |
| 16 | GRN (DVECGEGHFCHDN*QTCCR) |
| 17 | PTGDS (SVVAPATDGGLN*LTSTFLR) |
| 18 | UMOD (QDFN*ITDISLLEHR) |
| 19 | AFM (DIENFN*STQK) |
| 20 | CD97 (WCPQNSSCVN*ATACR) |
| 21 | TMPRSS2 (LN*TSAGNVDIYK) |
| 22 | CD63 (NN*HTASILDR) |
| 23 | ACPP (FLN*ESYK) (P15309); |
| 24 | LOX (AEN*QTAPGEVPALSNLRPPSR) (P28300); |
| 25 | CLU (EDALN*ETR) (P10909); |
| 26 | SERPINA1 (YLGN*ATAIFFLPDEGK) (P01009); |
| 27 | ORM1 (QDQCIYN*TTYLNVQR) (P02763); |
| 28 | CD63 (CCGAAN*YTDWEK) (P08962); |
| 29 | ATRN (ISN*SSDTVECECSENWK) (075882); |
| 30 | GP2 (QDLN*SSDVHSLQPQLDCGPR) (P55259); |
| 31 | KLK11 (TATESFPHPGFN*NSLPNK) (Q9UBX7); |
| 32 | PTPRN2 (VSANVQN*VTTEDVEK) (Q92932); |
| 33 | NPTN (AN*ATIEVK) (Q9Y639); |
| 34 | CPE (DLQGNPIAN*ATISVEGIDHDVTSAK) (P16870); |
| 35 | RNASE2 (NQNTFLLTTFANVVNVCGNPN*MTCPSN*K) |
| | (P10153); |
| 36 | DSC2 (NGIYN*ITVLASDQGGR) (Q02487); |
| 37 | LRG1 (LPPGLLAN*FTLLR) (P02750); |
| 38 | GRN (DVECGEGHFCHDN*QTCCR) (P28799); |
| 39 | PTGDS (SVVAPATDGGLN*LTSTFLR) (P41222); |
| 40 | UMOD (QDFN*ITDISLLEHR) (P07911); |
| 41 | AFM (DIENFN*STQK) (P43652); |
| 42 | CD97 (WCPQNSSCVN*ATACR) (P48960) |
| 43 | TMPRSS2 (LN*TSAGNVDIYK) (015393) |

1. A method comprising the step of measuring one or more of prostatic acid phosphatase (ACPP), CD63 antigen (CD63), kallikrein-11 (KLK11), attractin (ATRN), pancreatic secretory granule membrane major glycoprotein GP2 (GP2), receptor-type tyrosine-protein phosphatase N2 (PTPRN2), neuroplastin (NPTN), non-secretory ribonuclease (RNASE2), prostate-specific antigen (PSA), carboxypeptidase E (CPE), alpha-1-antitrypsin (SERPINA1), desmocollin-2 (DSC2), prostaglandin-H2 D9 isomerase (PTGDS), progranulin (GRN), leucine-rich alpha-2-glycprotein (LRG1), uromodulin (UMOD), clusterin (CLU), protein-lysine 6-oxidase (LOX), alpha-1-acid glycoprotein 1 (ORM1), CD97 antigen (CD97), Afamin (AFM) in a urine sample obtained from a subject having or suspected of having prostate cancer.

2. The method of claim 1, wherein the measuring step comprises an immunoassay.

3. The method of claim 2, wherein the immunoassay comprises enzyme linked immunosorbent assay (ELISA).

4. The method of claim 1, wherein the measuring step comprises mass spectrometry.

5. A method for identifying a subject as having aggressive prostate cancer comprising the step of measuring one or more of ACPP, CD63KLK 11, ATRN, GP2, PTPRN2, NPTN, RNASE2, PSA, CPE, SERPINA1, DSC2, PTGDS, GRN, LRG1, UMOD , CLU, LOX, ORM1, CD97, and AFM in a urine sample obtained from the subject, wherein a decreased level of one or more of ACPP, CD63, KLK 11 ATRN, GP2, PTPRN2, NPTN, RNASE2, PSA, CPE and/or an increased level of one or more of SERPINA1, DSC2, PTGDS, GRN, LRG1, UMOD, CLU, LOX, ORM1, CD97, and AFM relative to a control identifies the subject as having aggressive prostate cancer.

6. The method of claim 5, further comprising the step of treating the subject with a prostate cancer therapy.

7. A method comprising the steps of:
(a) detecting a decreased level of one or more of ACPP, CD63, KLK 11, ATRN, GP2, PTPRN2, NPTN, RNASE2, PSA, and CPE and/or an increased level of one or more of SERPINA1, DSC2, PTGDS, GRN, LRG1, UMOD, CLU, LOX, ORM1, CD97, and AFM, relative to a control in a urine sample obtained from a subject having or suspected of having prostate cancer; and
(b) treating the subject with a prostate cancer therapy.

8. The method of claim 7, wherein the prostate cancer therapy comprises prostatectomy, radiation therapy, cryotherapy, hormone therapy, chemotherapy, immunotherapy and combinations thereof.

9. The method of claim 7, wherein the detecting step comprises an immunoassay.

10. The method of claim 9, wherein the immunoassay comprises enzyme linked immunosorbent assay (ELISA).

11. The method of claim 7, wherein the detecting step comprises mass spectrometry.

12. A method for identifying a subject as having aggressive prostate cancer comprising the steps of:
(a) measuring ACPP and one or more of clusterin (CLU), protein-lysine 6-oxidase (LOX), alpha-1-antitrypsin (SERPINA1), and alpha-1-acid glycoprotein 1 (ORM1), in a urine sample obtained from the subject; and
(b) correlating the levels measured in step (a) with serum PSA to identify the subject as having aggressive prostate cancer.

13. The method of claim 12, further comprising the step of treating the subject with a prostate cancer therapy.

14. The method of claim 13, wherein the prostate cancer therapy comprises prostatectomy, radiation therapy, cryotherapy, hormone therapy, chemotherapy, immunotherapy and combinations thereof.

15. A method comprising the step of measuring (a) urine ACPP and serum PSA;
(b) urine ACPP and urine CLU; (c) urine ACPP and urine LOX; (d) urine ACPP and urine SERPINA1; (e) urine ACPP and urine ORM1; (f) ACPP, urine CLU and serum PSA; (g) urine ACPP, urine LOX and serum PSA; (h) urine ACPP, urine SERPINA1 and serum PSA; or (i) urine ACPP, urine ORM1 and serum PSA, in samples obtained from a subject having or suspected of having prostate cancer.

16. The method of claim 15, wherein the subject is identified as having aggressive prostate cancer or non-aggressive prostate cancer based on the measured levels.

17. A method comprising the step of measuring (a) ACPP; (b) ACPP and CLU; (c) ACPP and LOX; (d) ACPP and SERPINA1; (e) ACPP and ORM1; (f) ACPP and CLU; (g) ACPP and LOX; (h) ACPP and SERPINA1; or (i) ACPP and ORM1, in a urine sample obtained from a subject having or suspected of having prostate cancer.

18. The method of claim 17, wherein the measured proteins are used with serum PSA to diagnose the subject as having aggressive prostate cancer or non-aggressive prostate cancer.

19. The method of claim 18, wherein the subject having aggressive prostate cancer is treated with a prostate cancer therapy.

20. A method comprising the step of measuring ACPP, CLU, ORM1, CD97, and/or PSA in a urine sample obtained from a subject having or suspected of having prostate cancer.

21. The method of claim 20, wherein the measured proteins are used with combination of any two, three, four, or five of them to diagnose the subject as having aggressive prostate cancer or non-aggressive prostate cancer.

22. The method of claim 21, wherein the subject having aggressive prostate cancer is treated with a prostate cancer therapy.

23. The method of claim 22, wherein the prostate cancer therapy comprises prostatectomy, radiation therapy, cryotherapy, hormone therapy, chemotherapy, immunotherapy and combinations thereof.


SEQUENCE LISTING (43 sequences)

SEQ ID NO: 1 - protein, 7 aa, Homo sapiens
  MISC_FEATURE (1)..(7): Prostatic acid phosphatase (ACPP) peptide
FLNESYK

SEQ ID NO: 2 - protein, 21 aa, Homo sapiens
  MISC_FEATURE (1)..(21): Protein-lysine 6-oxidase (LOX) peptide
AENQTAPGEVPALSNLRPPSR

SEQ ID NO: 3 - protein, 8 aa, Homo sapiens
  MISC_FEATURE (1)..(8): Clusterin (CLU) peptide
EDALNETR

SEQ ID NO: 4 - protein, 16 aa, Homo sapiens
  MISC_FEATURE (1)..(16): Alpha-1-antitrypsin (SERPINA1) peptide
YLGNATAIFFLPDEGK

SEQ ID NO: 5 - protein, 15 aa, Homo sapiens
  MISC_FEATURE (1)..(15): Alpha-1-acid glycoprotein 1 (ORM1)
QDQCIYNTTYLNVQR

SEQ ID NO: 6 - protein, 12 aa, Homo sapiens
  MISC_FEATURE (1)..(12): CD63 antigen (CD63) peptide
CCGAANYTDWEK

SEQ ID NO: 7 - protein, 17 aa, Homo sapiens
  MISC_FEATURE (1)..(17): Attractin (ATRN) peptide
ISNSSDTVECECSENWK

SEQ ID NO: 8 - protein, 20 aa, Homo sapiens
  MISC_FEATURE (1)..(20): Pancreatic secretory granule membrane major glycoprotein GP2 (GP2) peptide
QDLNSSDVHSLQPQLDCGPR

SEQ ID NO: 9 - protein, 18 aa, Homo sapiens
  MISC_FEATURE (1)..(18): Kallikrein-11 (KLK11) peptide
TATESFPHPGFNNSLPNK

SEQ ID NO: 10 - protein, 15 aa, Homo sapiens
  MISC_FEATURE (1)..(15): Receptor-type tyrosine-protein phosphatase N2 (PTPRN2) peptide
VSANVQNVTTEDVEK

SEQ ID NO: 11 - protein, 8 aa, Homo sapiens
  MISC_FEATURE (1)..(8): Neuroplastin (NPTN) peptide
ANATIEVK

SEQ ID NO: 12 - protein, 25 aa, Homo sapiens
  MISC_FEATURE (1)..(25): Carboxypeptidase E (CPE) peptide
DLQGNPIANATISVEGIDHDVTSAK

SEQ ID NO: 13 - protein, 28 aa, Homo sapiens
  MISC_FEATURE (1)..(28): Non-secretory ribonuclease (RNASE2) peptide
NQNTFLLTTFANVVNVCGNPNMTCPSNK

SEQ ID NO: 14 - protein, 16 aa, Homo sapiens
  MISC_FEATURE (1)..(16): Desmocollin-2 (DSC2) peptide
NGIYNITVLASDQGGR

SEQ ID NO: 15 - protein, 13 aa, Homo sapiens
  MISC_FEATURE (1)..(13): Leucine-rich alpha-2-glycoprotein (LRG1) peptide
LPPGLLANFTLLR

SEQ ID NO: 16 - protein, 18 aa, Homo sapiens
  MISC_FEATURE (1)..(18): Progranulin (GRN) peptide
DVECGEGHFCHDNQTCCR

SEQ ID NO: 17 - protein, 19 aa, Homo sapiens
  MISC_FEATURE (1)..(19): Prostaglandin-H2 D9 isomerase (PTGDS) peptide
SVVAPATDGGLNLTSTFLR

SEQ ID NO: 18 - protein, 14 aa, Homo sapiens
  MISC_FEATURE (1)..(14): Uromodulin (UMOD) peptide
QDFNITDISLLEHR

SEQ ID NO: 19 - protein, 10 aa, Homo sapiens
  MISC_FEATURE (1)..(10): Afamin (AFM) peptide
DIENFNSTQK

SEQ ID NO: 20 - protein, 15 aa, Homo sapiens
  MISC_FEATURE (1)..(15): CD97 antigen (CD97) peptide
WCPQNSSCVNATACR

SEQ ID NO: 21 - protein, 12 aa, Homo sapiens
  MISC_FEATURE (1)..(12): Transmembrane serine protease 2 (TMPRSS2) peptide
LNTSAGNVDIYK

SEQ ID NO: 22 - protein, 10 aa, Homo sapiens
  MISC_FEATURE (1)..(10): CD63 antigen (CD63) peptide
NNHTASILDR

SEQ ID NO: 23 - protein, 386 aa, Homo sapiens
  MISC_FEATURE (1)..(238): ACPP (UniProt No. P15309)
  PEPTIDE (92)..(98)
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 24 - protein, 417 aa, Homo sapiens
  MISC_FEATURE (1)..(417): LOX (UniProt No. P28300)
  PEPTIDE (142)..(162)
MRFAWTVLLLGPLQLCALVHCAPPAAGQQQPPREPPAAPGAWRQQIQWENNGQVFSLLSLGSQYQPQRRRDPGAAVPGAANASAQQPRTPILLIRDNRTAAARTRTAGSSGVTAGRPRPTARHWFQAGYSTSRAREAGASRAENQTAPGEVPALSNLRPPSRVDGMVGDDPYNPYKYSDDNPYYNYYDTYERPRPGGRYRPGYGTGYFQYGLPDLVADPYYIQASTYVQKMSMYNLRCAAEENCLASTAYRADVRDYDHRVLLRFPQRVKNQGTSDFLPSRPRYSWEWHSCHQHYHSMDEFSHYDLLDANTQRRVAEGHKASFCLEDTSCDYGYHRRFACTAHTQGLSPGCYDTYGADIDCQWIDITDVKPGNYILKVSVNPSYLVPESDYTNNVVRCDIRYTGHHAYASGCTISPY

SEQ ID NO: 25 - protein, 449 aa, Homo sapiens
  MISC_FEATURE (1)..(449): CLU (UniProt No. P10909)
  PEPTIDE (82)..(89)
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 26 - protein, 418 aa, Homo sapiens
  MISC_FEATURE (1)..(418): SERPINA1 (UniProt No. P01009)
  PEPTIDE (268)..(283)
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 27 - protein, 200 aa, Homo sapiens
  MISC_FEATURE (1)..(200): ORM1 (UniProt No. P02763)
  PEPTIDE (87)..(101)
MALSWVLTVLSLLPLLEAQIPLCANLVPVPITNATLDQITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGE

SEQ ID NO: 28 - protein, 238 aa, Homo sapiens
  MISC_FEATURE (1)..(238): CD63 (Uniprot No. P08962)
  PEPTIDE (129)..(138)
  PEPTIDE (145)..(156)
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 29 - protein, 1429 aa, Homo sapiens
  MISC_FEATURE (1)..(1429): ATRN (UniProt No. O75882)
  PEPTIDE (262)..(278)
MVAAAAATEARLRRRTAATAALAGRSGGPHWDWDVTRAGRPGLGAGLRLPRLLSPPLRPRLLLLLLLLSPPLLLLLLPCEAEAAAAAAAVSGSAAAEAKECDRPCVNGGRCNPGTGQCVCPAGWVGEQCQHCGGRFRLTGSSGFVTDGPGNYKYKTKCTWLIEGQPNRIMRLRFNHFATECSWDHLYVYDGDSIYAPLVAAFSGLIVPERDGNETVPEVVATSGYALLHFFSDAAYNLTGFNITYSFDMCPNNCSGRGECKISNSSDTVECECSENWKGEACDIPHCTDNCGFPHRGICNSSDVRGCSCFSDWQGPGCSVPVPANQSFWTREEYSNLKLPRASHKAVVNGNIMWVVGGYMFNHSDYNMVLAYDLASREWLPLNRSVNNVVVRYGHSLALYKDKIYMYGGKIDSTGNVTNELRVFHIHNESWVLLTPKAKEQYAVVGHSAHIVTLKNGRVVMLVIFGHCPLYGYISNVQEYDLDKNTWSILHTQGALVQGGYGHSSVYDHRTRALYVHGGYKAFSANKYRLADDLYRYDVDTQMWTILKDSRFFRYLHTAVIVSGTMLVFGGNTHNDTSMSHGAKCFSSDFMAYDIACDRWSVLPRPDLHHDVNRFGHSAVLHNSTMYVFGGFNSLLLSDILVFTSEQCDAHRSEAACLAAGPGIRCVWNTGSSQCISWALATDEQEEKLKSECFSKRTLDHDRCDQHTDCYSCTANTNDCHWCNDHCVPRNHSCSEGQISIFRYENCPKDNPMYYCNKKTSCRSCALDQNCQWEPRNQECIALPENICGIGWHLVGNSCLKITTAKENYDNAKLFCRNHNALLASLTTQKKVEFVLKQLRIMQSSQSMSKLTLTPWVGLRKINVSYWCWEDMSPFTNSLLQWMPSEPSDAGFCGILSEPSTRGLKAATCINPLNGSVCERPANHSAKQCRTPCALRTACGDCTSGSSECMWCSNMKQCVDSNAYVASFPFGQCMEWYTMSTCPPENCSGYCTCSHCLEQPGCGWCTDPSNTGKGKCIEGSYKGPVKMPSQAPTGNFYPQPLLNSSMCLEDSRYNWSFIHCPACQCNGHSKCINQSICEKCENLTTGKHCETCISGFYGDPTNGGKCQPCKCNGHASLCNTNTGKCFCTTKGVKGDECQLCEVENRYQGNPLRGTCYYTLLIDYQFTFSLSQEDDRYYTAINFVATPDEQNRDLDMFINASKNFNLNITWAASFSAGTQAGEEMPVVSKTNIKEYKDSFSNEKFDFRNHPNITFFVYVSNFTWPIKIQIAFSQHSNFMDLVQFFVTFFSCFLSLLLVAAVVWKIKQSCWASRRREQLLREMQQMASRPFASVNVALETDEEPPDLIGGSIKTVPKPIALEPCFGNKAAVLSVFVRLPRGLGGIPPPGQSGLAVASALVDISQQMPIVYKEKSGAVRNRKQQPPAQPGTCI

SEQ ID NO: 30 - protein, 537 aa, Homo sapiens
  MISC_FEATURE (1)..(537): GP2 (UniProt No. P55259)
  PEPTIDE (213)..(232)
MPHLMERMVGSGLLWLALVSCILTQASAVQRGYGNPIEASSYGLDLDCGAPGTPEAHVCFDPCQNYTLLDEPFRSTENSAGSQGCDKNMSGWYRFVGEGGVRMSETCVQVHRCQTDAPMWLNGTHPALGDGITNHTACAHWSGNCCFWKTEVLVKACPGGYHVYRLEGTPWCNLRYCTVPRDPSTVEDKCEKACRPEEECLALNSTWGCFCRQDLNSSDVHSLQPQLDCGPREIKVKVDKCLLGGLGLGEEVIAYLRDPNCSSILQTEERNWVSVTSPVQASACRNILERNQTHAIYKNTLSLVNDFIIRDTILNINFQCAYPLDMKVSLQAALQPIVSSLNVSVDGNGEFIVRMALFQDQNYTNPYEGDAVELSVESVLYVGAILEQGDTSRFNLVLRNCYATPTEDKADLVKYFIIRNSCSNQRDSTIHVEENGQSSESRFSVQMFMFAGHYDLVFLHCEIHLCDSLNEQCQPSCSRSQVRSEVPAIDLARVLDLGPITRRGAQSPGVMNGTPSTAGFLVAWPMVLLTVLLAWLF

SEQ ID NO: 31 - protein, 282 aa, Homo sapiens
  MISC_FEATURE (1)..(282): KLK11 (UniProt No. Q9UBX7)
  PEPTIDE (120)..(137)
MQRLRWLRDWKSSGRGLTAAKEPGARSSPLQAMRILQLILLALATGLVGGETRIIKGFECKPHSQPWQAALFEKTRLLCGATLIAPRWLLTAAHCLKPRYIVHLGQHNLQKEEGCEQTRTATESFPHPGFNNSLPNKDHRNDIMLVKMASPVSITWAVRPLTLSSRCVTAGTSCLISGWGSTSSPQLRLPHTLRCANITIIEHQKCENAYPGNITDTMVCASVQEGGKDSCQGDSGGPLVCNQSLQGIISWGQDPCAITRKPGVYTKVCKYVDWIQETMKNN

SEQ ID NO: 32 - protein, 1015 aa, Homo sapiens
  MISC_FEATURE (1)..(1015): PTPRN2 (UniProt No. Q92932)
  PEPTIDE (558)..(572)
MGPPLPLLLLLLLLLPPRVLPAAPSSVPRGRQLPGRLGCLLEEGLCGASEACVNDGVFGRCQKVPAMDFYRYEVSPVALQRLRVALQKLSGTGFTWQDDYTQYVMDQELADLPKTYLRRPEASSPARPSKHSVGSERRYSREGGAALANALRRHLPFLEALSQAPASDVLARTHTAQDRPPAEGDDRFSESILTYVAHTSALTYPPGSRTQLREDLLPRTLGQLQPDELSPKVDSGVDRHHLMAALSAYAAQRPPAPPGEGSLEPQYLLRAPSRMPRPLLAPAAPQKWPSPLGDSEDPSSTGDGARIHTLLKDLQRQPAEVRGLSGLELDGMAELMAGLMQGVDHGVARGSPGRAALGESGEQADGPKATLRGDSFPDDGVQDDDDRLYQEVHRLSATLGGLLQDHGSRLLPGALPFARPLDMERKKSEHPESSLSSEEETAGVENVKSQTYSKDLLGQQPHSEPGAAAFGELQNQMPGPSKEEQSLPAGAQEALSDGLQLEVQPSEEEARGYIVTDRDPLRPEEGRRLVEDVARLLQVPSSAFADVEVLGPAVTFKVSANVQNVTTEDVEKATVDNKDKLEETSGLKILQTGVGSKSKLKFLPPQAEQEDSTKFIALTLVSLACILGVLLASGLIYCLRHSSQHRLKEKLSGLGGDPGADATAAYQELCRQRMATRPPDRPEGPHTSRISSVSSQFSDGPIPSPSARSSASSWSEEPVQSNMDISTGHMILSYMEDHLKNKNRLEKEWEALCAYQAEPNSSFVAQREENVPKNRSLAVLTYDHSRVLLKAENSHSHSDYINASPIMDHDPRNPAYIATQGPLPATVADFWQMVWESGCVVIVMLTPLAENGVRQCYHYWPDEGSNLYHIYEVNLVSEHIWCEDFLVRSFYLKNLQTNETRTVTQFHFLSWYDRGVPSSSRSLLDFRRKVNKCYRGRSCPIIVHCSDGAGRSGTYVLIDMVLNKMAKGAKEIDIAATLEHLRDQRPGMVQTKEQFEFALTAVAEEVNAILKALPQ

SEQ ID NO: 33 - protein, 398 aa, Homo sapiens
  MISC_FEATURE (1)..(398): NPTN (UniProt No. Q9Y639)
  PEPTIDE (228)..(235)
MSGSSLPSALALSLLLVSGSLLPGPGAAQNAGFVKSPMSETKLTGDAFELYCDVVGSPTPEIQWWYAEVNRAESFRQLWDGARKRRVTVNTAYGSNGVSVLRITRLTLEDSGTYECRASNDPKRNDLRQNPSITWIRAQATISVLQKPRIVTSEEVIIRDSPVLPVTLQCNLTSSSHTLTYSYWTKNGVELSATRKNASNMEYRINKPRAEDSGEYHCVYHFVSAPKANATIEVKAAPDITGHKRSENKNEGQDATMYCKSVGYPHPDWIWRKKENGMPMDIVNTSGRFFIINKENYTELNIVNLQITEDPGEYECNATNAIGSASVVTVLRVRSHLAPLWPFLGILAEIIILVVIIVVYEKRKRPDEVPDDDEPAGPMKTNSTNNHKDKNLRQRNTN

SEQ ID NO: 34 - protein, 476 aa, Homo sapiens
  MISC_FEATURE (1)..(476): CPE (UniProt No. P16870)
  PEPTIDE (382)..(406)
MAGRGGSALLALCGALAACGWLLGAEAQEPGAPAAGMRRRRRLQQEDGISFEYHRYPELREALVSVWLQCTAISRIYTVGRSFEGRELLVIELSDNPGVHEPGEPEFKYIGNMHGNEAVGRELLIFLAQYLCNEYQKGNETIVNLIHSTRIHIMPSLNPDGFEKAASQPGELKDWFVGRSNAQGIDLNRNFPDLDRIVYVNEKEGGPNNHLLKNMKKIVDQNTKLAPETKAVIHWIMDIPFVLSANLHGGDLVANYPYDETRSGSAHEYSSSPDDAIFQSLARAYSSFNPAMSDPNRPPCRKNDDDSSFVDGTTNGGAWYSVPGGMQDFNYLSSNCFEITVELSCEKFPPEETLKTYWEDNKNSLISYLEQIHRGVKGFVRDLQGNPIANATISVEGIDHDVTSAKDGDYWRLLIPGNYKLTASAPGYLAITKKVAVPYSPAAGVDFELESFSERKEEEKEELMEWWKMMSETLNF

SEQ ID NO: 35 - protein, 161 aa, Homo sapiens
  MISC_FEATURE (1)..(161): RNASE2 (UniProt No. P10153)
  PEPTIDE (66)..(93)
MVPKLFTSQICLLLLLGLLAVEGSLHVKPPQFTWAQWFETQHINMTSQQCTNAMQVINNYQRRCKNQNTFLLTTFANVVNVCGNPNMTCPSNKTRKNCHHSGSQVPLIHCNLTTPSPQNISNCRYAQTPANMFYIVACDNRDQRRDPPQYPVVPVHLDRII

SEQ ID NO: 36 - protein, 901 aa, Homo sapiens
  MISC_FEATURE (1)..(901): DSC2 (UniProt No. Q02487)
  PEPTIDE (542)..(557)
MEAARPSGSWNGALCRLLLLTLAILIFASDACKNVTLHVPSKLDAEKLVGRVNLKECFTAANLIHSSDPDFQILEDGSVYTTNTILLSSEKRSFTILLSNTENQEKKKIFVFLEHQTKVLKKRHTKEKVLRRAKRRWAPIPCSMLENSLGPFPLFLQQVQSDTAQNYTIYYSIRGPGVDQEPRNLFYVERDTGNLYCTRPVDREQYESFEIIAFATTPDGYTPELPLPLIIKIEDENDNYPIFTEETYTFTIFENCRVGTTVGQVCATDKDEPDTMHTRLKYSIIGQVPPSPTLFSMHPTTGVITTTSSQLDRELIDKYQLKIKVQDMDGQYFGLQTTSTCIINIDDVNDHLPTFTRTSYVTSVEENTVDVEILRVTVEDKDLVNTANWRANYTILKGNENGNFKIVTDAKTNEGVLCVVKPLNYEEKQQMILQIGVVNEAPFSREASPRSAMSTATVTVNVEDQDEGPECNPPIQTVRMKENAEVGTTSNGYKAYDPETRSSSGIRYKKLTDPTGWVTIDENTGSIKVFRSLDREAETIKNGIYNITVLASDQGGRTCTGTLGIILQDVNDNSPFIPKKTVIICKPTMSSAEIVAVDPDEPIHGPPFDFSLESSTSEVQRMWRLKAINDTAARLSYQNDPPFGSYVVPITVRDRLGMSSVTSLDVTLCDCITENDCTHRVDPRIGGGGVQLGKWAILAILLGIALLFCILFTLVCGASGTSKQPKVIPDDLAQQNLIVSNTEAPGDDKVYSANGFTTQTVGASAQGVCGTVGSGIKNGGQETIEMVKGGHQTSESCRGAGHHHTLDSCRGGHTEVDNCRYTYSEWHSFTQPRLGEKVYLCNQDENHKHAQDYVLTYNYEGRGSVAGSVGCCSERQEEDGLEFLDNLEPKFRTLAEACMKR

SEQ ID NO: 37 - protein, 347 aa, Homo sapiens
  MISC_FEATURE (1)..(347): LRG1 (UniProt No. P02750)
  PEPTIDE (179)..(191)
MSSWSRQRPKSPGGIQPHVSRTLFLLLLLAASAWGVTLSPKDCQVFRSDHGSSISCQPPAEIPGYLPADTVHLAVEFFNLTHLPANLLQGASKLQELHLSSNGLESLSPEFLRPVPQLRVLDLTRNALTGLPPGLFQASATLDTLVLKENQLEVLEVSWLHGLKALGHLDLSGNRLRKLPPGLLANFTLLRTLDLGENQLETLPPDLLRGPLQLERLHLEGNKLQVLGKDLLLPQPDLRYLFLNGNKLARVAAGAFQGLRQLDMLDLSNNSLASVPEGLWASLGQPNWDMRDGFDISGNPWICDQNLSDLYRWLQAQKDKMFSQNDTRCAGPEAVKGQTLLAVAKSQ

SEQ ID NO: 38 - protein, 593 aa, Homo sapiens
  MISC_FEATURE (1)..(593): GRN (UniProt No. P28799)
  PEPTIDE (518)..(535)
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 39 - protein, 190 aa, Homo sapiens
  MISC_FEATURE (1)..(190): PTGDS (UniProt No. P41222)
  PEPTIDE (67)..(85)
MATHHTLWMGLALLGVLGDLQAAPEAQVSVQPNFQQDKFLGRWFSAGLASNSSWLREKKAALSMCKSVVAPATDGGLNLTSTFLRKNQCETRTMLLQPAGSLGSYSYRSPHWGSTYSVSVVETDYDQYALLYSQGSKGPGEDFRMATLYSRTQTPRAELKEKFTAFCKAQGFTEDTIVFLPQTDKCMTEQ

SEQ ID NO: 40 - protein, 640 aa, Homo sapiens
  MISC_FEATURE (1)..(640): UMOD (UniProt No. P07911)
  PEPTIDE (319)..(332)
MGQPSLTWMLMVVVASWFITTAATDTSEARWCSECHSNATCTEDEAVTTCTCQEGFTGDGLTCVDLDECAIPGAHNCSANSSCVNTPGSFSCVCPEGFRLSPGLGCTDVDECAEPGLSHCHALATCVNVVGSYLCVCPAGYRGDGWHCECSPGSCGPGLDCVPEGDALVCADPCQAHRTLDEYWRSTEYGEGYACDTDLRGWYRFVGQGGARMAETCVPVLRCNTAAPMWLNGTHPSSDEGIVSRKACAHWSGHCCLWDASVQVKACAGGYYVYNLTAPPECHLAYCTDPSSVEGTCEECSIDEDCKSNNGRWHCQCKQDFNITDISLLEHRLECGANDMKVSLGKCQLKSLGFDKVFMYLSDSRCSGFNDRDNRDWVSVVTPARDGPCGTVLTRNETHATYSNTLYLADEIIIRDLNIKINFACSYPLDMKVSLKTALQPMVSALNIRVGGTGMFTVRMALFQTPSYTQPYQGSSVTLSTEAFLYVGTMLDGGDLSRFALLMTNCYATPSSNATDPLKYFIIQDRCPHTRDSTIQVVENGESSQGRFSVQMFRFAGNYDLVYLHCEVYLCDTMNEKCKPTCSGTRFRSGSVIDQSRVLNLGPITRKGVQATVSRAFSSLGLLKVWLPLLLSATLTLTFQ

SEQ ID NO: 41 - protein, 599 aa, Homo sapiens
  MISC_FEATURE (1)..(599): AFM (UniProt No. P43652)
  PEPTIDE (28)..(37)
MKLLKLTGFIFFLFFLTESLTLPTQPRDIENFNSTQKFIEDNIEYITIIAFAQYVQEATFEEMEKLVKDMVEYKDRCMADKTLPECSKLPNNVLQEKICAMEGLPQKHNFSHCCSKVDAQRRLCFFYNKKSDVGFLPPFPTLDPEEKCQAYESNRESLLNHFLYEVARRNPFVFAPTLLTVAVHFEEVAKSCCEEQNKVNCLQTRAIPVTQYLKAFSSYQKHVCGALLKFGTKVVHFIYIAILSQKFPKIEFKELISLVEDVSSNYDGCCEGDVVQCIRDTSKVMNHICSKQDSISSKIKECCEKKIPERGQCIINSNKDDRPKDLSLREGKFTDSENVCQERDADPDTFFAKFTFEYSRRHPDLSIPELLRIVQIYKDLLRNCCNTENPPGCYRYAEDKFNETTEKSLKMVQQECKHFQNLGKDGLKYHYLIRLTKIAPQLSTEELVSLGEKMVTAFTTCCTLSEEFACVDNLADLVFGELCGVNENRTINPAVDHCCKTNFAFRRPCFESLKADKTYVPPPFSQDLFTFHADMCQSQNEELQRKTDRFLVNLVKLKHELTDEELQSLFTNFANVVDKCCKAESPEVCFNEESPKIGN

SEQ ID NO: 42 - protein, 835 aa, Homo sapiens
  MISC_FEATURE (1)..(835): CD97 (UniProt No. P48960)
  PEPTIDE (29)..(43)
MGGRVFLAFCVWLTLPGAETQDSRGCARWCPQNSSCVNATACRCNPGFSSFSEIITTPTETCDDINECATPSKVSCGKFSDCWNTEGSYDCVCSPGYEPVSGAKTFKNESENTCQDVDECQQNPRLCKSYGTCVNTLGSYTCQCLPGFKFIPEDPKVCTDVNECTSGQNPCHSSTHCLNNVGSYQCRCRPGWQPIPGSPNGPNNTVCEDVDECSSGQHQCDSSTVCFNTVGSYSCRCRPGWKPRHGIPNNQKDTVCEDMTFSTWTPPPGVHSQTLSRFFDKVQDLGRDSKTSSAEVTIQNVIKLVDELMEAPGDVEALAPPVRHLIATQLLSNLEDIMRILAKSLPKGPFTYISPSNTELTLMIQERGDKNVTMGQSSARMKLNWAVAAGAEDPGPAVAGILSIQNMTTLLANASLNLHSKKQAELEEIYESSIRGVQLRRLSAVNSIFLSHNNTKELNSPILFAFSHLESSDGEAGRDPPAKDVMPGPRQELLCAFWKSDSDRGGHWATEGCQVLGSKNGSTTCQCSHLSSFAILMAHYDVEDWKLTLITRVGLALSLFCLLLCILTFLLVRPIQGSRTTIHLHLCICLFVGSTIFLAGIENEGGQVGLRCRLVAGLLHYCFLAAFCWMSLEGLELYFLVVRVFQGQGLSTRWLCLIGYGVPLLIVGVSAAIYSKGYGRPRYCWLDFEQGFLWSFLGPVTFIILCNAVIFVTTVWKLTQKFSEINPDMKKLKKARALTITAIAQLFLLGCTWVFGLFIFDDRSLVLTYVFTILNCLQGAFLYLLHCLLNKKVREEYRKWACLVAGGSKYSEFTSTTSGTGHNQTRALRASESGI

SEQ ID NO: 43 - protein, 492 aa, Homo sapiens
  MISC_FEATURE (1)..(492): TMPRSS2 (UniProt No. O15393)
  PEPTIDE (212)..(223)
MALNSGSPPAIGPYYENHGYQPENPYPAQPTVVPTVYEVHPAQYYPSPVPQYAPRVLTQASNPVVCTQPKSPSGTVCTSKTKKALCITLTLGTFLVGAALAAGLLWKFMGSKCSNSGIECDSSGTCINPSNWCDGVSHCPGGEDENRCVRLYGPNFILQVYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADG